SEQUENCE LISTING (79 sequences)

SEQ ID NO: 1 - DNA, 132 bp, Artificial Sequence
  synthetic construct
gatgctcttgacgattttgacctcgatatgctcgacgctcttgatgattttgatctcgacatgctcgatgcacttgatgactttgaccttgacatgctcgacgcactcgatgacttcgacctcgacatgctt

SEQ ID NO: 2 - protein, 44 aa, Artificial Sequence
  synthetic construct
DALDDFDLDMLDALDDFDLDMLDALDDFDLDMLDALDDFDLDML

SEQ ID NO: 3 - DNA, 153 bp, Artificial Sequence
  synthetic construct
ctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacag

SEQ ID NO: 4 - protein, 51 aa, Artificial Sequence
  synthetic construct
LLDPGTPMDADLVASSTVVWEQDADPFAGTADDFPAFNEEELAWLMELLPQ

SEQ ID NO: 5 - DNA, 321 bp, Artificial Sequence
  synthetic construct
ctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcag

SEQ ID NO: 6 - protein, 107 aa, Artificial Sequence
  synthetic construct
LLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQ

SEQ ID NO: 7 - DNA, 504 bp, Artificial Sequence
  synthetic construct
ctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagaagcttaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgctt

SEQ ID NO: 8 - protein, 168 aa, Artificial Sequence
  synthetic construct
LLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQKLKGDGSGSGSGSGSVDDALDDFDLDMLDALDDFDLDMLDALDDFDLDMLDALDDFDLDML

SEQ ID NO: 9 - DNA, 1005 bp, Artificial Sequence
  synthetic construct
ttgcttgatccggggacaccaatggacgcggacctggtggcttcatcgaccgtggtttgggaacaggacgccgatccattcgccgggaccgccgatgactttcctgcttttaatgaggaagagttggcttggctgatggaactcctgccgcagggcggctcagggggtctccttgaccccggcacccccatggacgctgacctcgttgcaagttcgacggttgtttgggagcaagatgcagatccgtttgcgggtacagctgatgactttccagccttcaacgaagaggagctggcatggcttatggagcttctgcctcaggccagaggagggtcgggtgggctcttggatcctgggacgccgatggatgccgaccttgtcgcgtcctctactgtggtttgggagcaggacgccgacccgttcgctggtactgctgatgactttccggcctttaatgaggaggaactcgcttggctcatggagttgttgccacaagggggtagtggcggtcttctggacccgggaacgcctatggatgctgacttggtggcatcgagcaccgtcgtctgggaacaggatgcggacccgtttgctggtaccgctgatgattttccggcatttaacgaggaagaattggcgtggctcatggaattgcttccccaggcgagagggggttccggtggcttgctggatccgggaacgcctatggatgccgatctcgtggcgtcgtcaaccgtggtctgggagcaagacgctgacccgtttgccggcacagcagatgattttccagctttcaatgaggaagaactggcatggttgatggaattgcttccacagggagggagcggaggcctcctcgatcctggaacacctatggacgcagacctggttgcctcttcaactgtcgtgtgggaacaagatgcagacccgttcgcaggtacagctgatgacttccctgcctttaatgaggaggaactcgcgtggctcatggaacttttgccccaa

SEQ ID NO: 10 - protein, 335 aa, Artificial Sequence
  synthetic construct
LLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQARGGSGGLLDPGTPMDADLVASSTVVWEQDADPFAGTADDFPAFNEEELAWLMELLPQGGSGGLLDPGTPMDADLVASSTVVWEQDADPFAGTADDFPAFNEEELAWLMELLPQ

SEQ ID NO: 11 - DNA, 1146 bp, Artificial Sequence
  synthetic construct
gatgcgcttgacgactttgacttggacatgttgggatcagatgccctcgacgactttgatctcgatatgcttggttcggacgcgcttgacgatttcgatcttgacatgctcggttctgatgcacttgatgacttcgatttggacatgctgcccaaaaagaaaagaaaggtggatccaaagaagaaacgcaaagtccctactcaggctggggaagggactctgagtgaagctctcctgcaacttcagttcgacgacgaggacttgggtgcccttctcgggaacagcaccgacccggctgttttcactgacctcgcgtctgttgataattccgaattccaacagcttttgaatcaaggaattcctgttgccccacatactactgaacccatgctgatggaatatcccgaagcaataactagactggtgaccggtgcgcagcggccgccagacccagctccagccccgctgggtgcaccaggactgccgaatggcttgctgtcaggcgatgaagactttagttcgatcgctgatatggacttctctgcactgcttattaattccaggtcttctggtagtcgggactctcgcgagggtatgttcttgccaaaaccagaggctggatcggcaatatctgatgtttttgagggacgggaggtctgccagccgaaacgcatccgcccgtttcacccgccaggtagcccctgggcaaaccgcccattgcccgcctcgcttgccccaacccccaccggtcctgttcacgaacccgtcggatctctgaccccagcccctgttccacagccgttggacccagccccagcagtcaccccggaggccagtcatctcctggaggacccagatgaagagaccagtcaggcagttaaggctttgcgggaaatggctgacaccgtcatacctcagaaagaagaagcagccatttgcggacagatggatctgtctcatccccctccccggggtcaccttgatgagctgacaacaacgctggagagtatgacggaagatcttaacttggactcacctctgaccccggaactcaacgaaatattggatacatttttgaacgatgagtgtctgcttcatgcaatgcacatcagtaccggactgagtatatttgatactagcttgttt

SEQ ID NO: 12 - protein, 382 aa, Artificial Sequence
  synthetic construct
DALDDFDLDMLGSDALDDFDLDMLGSDALDDFDLDMLGSDALDDFDLDMLPKKKRKVDPKKKRKVPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLINSRSSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 13 - DNA, 858 bp, Artificial Sequence
  synthetic construct
atggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctggg

SEQ ID NO: 14 - protein, 286 aa, Artificial Sequence
  synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGGSG

SEQ ID NO: 15 - DNA, 57 bp, Artificial Sequence
  synthetic construct
gaggagttgctgagcaaaaattatcatttggagaacgaagtagcacgactaaagaaa

SEQ ID NO: 16 - protein, 19 aa, Artificial Sequence
  synthetic construct
EELLSKNYHLENEVARLKK

SEQ ID NO: 17 - DNA, 633 bp, Artificial Sequence
  synthetic construct
gaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaag

SEQ ID NO: 18 - protein, 211 aa, Artificial Sequence
  synthetic construct
EELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKK

SEQ ID NO: 19 - DNA, 21 bp, Artificial Sequence
  synthetic construct
ccgaagaagaagaggaaggtt

SEQ ID NO: 20 - protein, 7 aa, Artificial Sequence
  synthetic construct
PKKKRKV

SEQ ID NO: 21 - DNA, 4101 bp, Artificial Sequence
  synthetic construct
gacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgac

SEQ ID NO: 22 - protein, 1367 aa, Artificial Sequence
  synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - DNA, 4101 bp, Artificial Sequence
  synthetic construct
gacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgagaggacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattaggcctaagcggaacagcgacaagctcatcgcccgcaagaaggactgggatccgaagaagtacggcgggttcctgtggcccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctaagcagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaggctcggcgcccctagggctttcaagtacttcgacacaactatcgatccgaagcagtacaggagcactaaggaggtcctggacgcgaccctcatccaccagtcgattaccggcctctacgagacgcgcatcgacctgtctcagctcgggggcgac

SEQ ID NO: 24 - protein, 1367 aa, Artificial Sequence
  synthetic construct
DKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 25 - DNA, 3384 bp, Artificial Sequence
  synthetic construct
gccgtcaagtccatgaaggtcaagttgcgcctggataacatgccagagatcagagccggactttggaaacttcacaccgaggttaatgcgggtgtgcggtactatacggaatggcttagccttttgaggcaagaaaatctttatcggaggagtcccaatggcgatggagaacaagaatgctataaaactgctgaggaatgcaaggctgaactccttgagagactcagagcccgccaagttgagaatgggcactgcggccctgctgggagtgatgacgaactgctgcaattggcacggcaactttatgaacttctggtcccacaagcaatcggggctaaaggtgatgcgcagcaaatcgcaaggaagtttcttagtccccttgccgacaaggatgccgtgggtggtttgggaatagcaaaagcaggaaataagcctaggtgggttcggatgagggaggctggagagccaggttgggaagaggaaaaggctaaagccgaggcgagaaagagtacggatagaaccgccgatgttcttcgcgctcttgcagacttcggtcttaaacctcttatgagagtctacacagactcagacatgtccagcgtgcagtggaaaccacttcgcaaaggacaagcggtcagaacctgggatagagacatgttccaacaagcgatcgaaagaatgatgagttgggaatcgtggaatcagcgcgttggagaagcgtacgcaaagctcgtggaacaaaagtcgaggtttgaacagaaaaattttgtgggacaagaacatcttgtccaacttgtcaatcaacttcaacaagacatgaaggaagcatcacacggcctggagtcgaaagaacaaactgcgcattacttgactgggagagcgctgagagggagcgacaaagtttttgagaagtgggaaaaactcgatcctgatgccccatttgacctctatgataccgaaatcaagaatgttcaacggaggaatactcgcaggttcggatctcatgatctgtttgcgaagctcgcggaacctaaatatcaggcgctctggagagaggacgcttctttcctcacgaggtatgcggtttacaatagcattgtcagaaaactgaatcacgctaaaatgtttgcgacttttactcttccggatgctaccgcccacccgatctggacgcggtttgacaaactcggcggcaacctgcaccagtacactttcttgtttaacgaatttggcgagggcaggcacgccattcggtttcagaagctgttgacggttgaggatggcgttgctaaagaggtcgacgacgtcacggttccgatttctatgtccgcgcagctggatgacctcttgcctcgggacccacacgagctcgttgcactctacttccaggactacggtgcagaacaacatctggctggagagtttggcggcgcgaaaattcaataccgccgcgatcaattgaaccacctgcacgccagaagaggcgccagagatgtctaccttaatctgagcgtccgcgttcagtcacaatccgaagccaggggagaaaggcgccctccgtatgcagcggtcttcaggcttgttggcgataaccaccgcgcgtttgttcactttgataaattgtcagattacctcgcagaacacccagacgatggtaagctggggtcggaaggtttgctctctgggctcagagtcatgtcagttgacttgggtcttaggacttccgcgagcatatctgtcttccgcgtcgcaagaaaggacgaattgaagccgaacagtgaaggccgggtccctttttgcttcccgatcgaagggaacgaaaacctcgttgctgtccacgagcggagccaactgttgaagcttcccggtgaaacggaatcgaaagatctgagagcgatcagagaagagcgccaaaggacgcttagacagctccggacgcaacttgcatacttgcgccttctggttcgctgcggtagtgaagacgttggaagaagagagaggtcatgggctaaactcatagagcaacctatggatgctaatcaaatgacgcctgattggagagaagcattcgaagacgaacttcagaaactgaaatccctttacgggatatgcggcgatcgcgagtggacagaagcagtgtatgagtctgtgaggcgcgtgtggcggcatatgggtaaacaggtgcgcgattggagaaaagacgttaggagcggggaaagacctaagatacggggatatcagaaagacgttgtcgggggaaatagcattgaacagattgaatatttggagcgccaatataagttcctcaaatcctggtctttcttcggcaaagtgtcaggccaggtgatacgcgcggaaaagggatcgcgctttgcaataactctgagagaacatattgatcatgccaaagaagatcggttgaagaaactcgccgatagaatcatcatggaggcgcttggttatgtctacgccttggacgatgaacggggaaagggaaagtgggtcgccaagtatccaccttgccaactcattctcctcgaagaactttccgaataccagtttaacaacgatcggccgccatcagagaataatcaactgatgcagtggtcccatcgcggtgtgtttcaagagttgctcaatcaggcccaagtccatgatctgcttgttggcacaatgtatgcagccttttcctcccggtttgatgcaagaacaggggctcctggcatacgctgtagacgggtcccggcgaggtgcgcccgcgaacaaaaccctgaaccgttcccctggtggttgaacaagttcgttgcggagcacaagctggacgggtgtcctctgcgggccgacgatcttattcccaccggggaaggggaattctttgtgagccctttctcggcggaggaaggggattttcaccaaatacatgcagatcttaatgccgcacaaaatttgcagaggagactgtggtcagactttgatattagtcagatacgcctccgctgtgactggggagaggtcgatggcgagcctgtgttgataccaagaacgaccggaaagaggacagccgattcgtatggaaacaaggttttttacacgaagacgggcgttacttactacgaaagagaaagagggaagaagagaaggaaagtctttgcccaagaagaattgagcgaggaagaagccgagctcttggtcgaagcggacgaggcacgggaaaagtctgtcgtcctcatgagggacccttccggaattattaaccggggagattggacgcggcagaaagagttttggtccatggttaatcaacgcatagaaggctaccttgtcaagcaaataagaagtcgcgtgagattgcaggagagtgcatgtgagaacactggggacata

SEQ ID NO: 26 - protein, 1128 aa, Artificial Sequence
  synthetic construct
AVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 27 - DNA, 377 bp, Artificial Sequence
  synthetic construct
aagggatctttaaacatacgaacagatcacttaaagttcttctgaagcaacttaaagttatcaggcatgcatggatcttggaggaatcagatgtgcagtcagggaccatagcacaggacaggcgtcttctactggtgctaccagcaaatgctggaagccgggaacactgggtacgttggaaaccacgtgatgtggagtaagataaactgtaggagaaaagcatttcgtagtgggccatgaagcctttcaggacatgtattgcagtatgggccggcccattacgcaattggacgacaacaaagactagtattagtaccacctcggctatccacatagatcaaagctggtttaaaagagttgtgcagatgatccgtggc

SEQ ID NO: 28 - DNA, 77 bp, Artificial Sequence
  synthetic construct
aacaaagcaccagtggtctagtggtagaatagtaccctgccacggtacagacccgggttcgattcccggctggtgca

SEQ ID NO: 29 - DNA, 1998 bp, Artificial Sequence
  synthetic construct
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 30 - DNA, 597 bp, Artificial Sequence
  synthetic construct
atggaggcctctccggcttcaggtcctcgccatctcatggacccccatatctttacctccaatttcaataacggcattggcaggcacaagacatacctctgttatgaggtcgagcggctcgataacggaacctcagtgaagatggaccaacacaggggtttccttcataaccaagcgaaaaatctcctttgcggtttctatgggcggcacgcggagctgagattcttggatttggtcccgagcctccagctggatcccgcgcaaatataccgcgtcacttggttcatatcatggagcccgtgctttagctgggggtgtgccggtgaggttcgcgcgttcctccaggaaaacacccatgttcgcctccggattttcgcagcgaggattttcgactatgatccactttacaaagaggccctccaaatgcttagagatgctggggctcaagtttcaattatgacatacgacgagtttaagcattgttgggacacttttgtcgatcatcagggttgtcccttccagccgtgggatggtcttgatgagcattcgcaagctttgagtggcaggcttcgcgcgatacttcagaaccagggtaac

SEQ ID NO: 31 - protein, 199 aa, Artificial Sequence
  synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 32 - DNA, 498 bp, Artificial Sequence
  synthetic construct
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagtcctccatcaac

SEQ ID NO: 33 - protein, 166 aa, Artificial Sequence
  synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 34 - DNA, 19 bp, Artificial Sequence
  synthetic construct
acatgaggatcacccatgt

SEQ ID NO: 35 - DNA, 390 bp, Artificial Sequence
  synthetic construct
atggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctat

SEQ ID NO: 36 - protein, 130 aa, Artificial Sequence
  synthetic construct
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 37 - DNA, 14077 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaaggagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 38 - DNA, 14716 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagtcctccatcaactccggaggatctagcggaggctcctctggctctgagacacctggcacaagcgagagcgcaacacctgaaagcagcgggggcagcagcggggggtcaaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaaggagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 39 - DNA, 14657 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcatggaggcctctccggcttcaggtcctcgccatctcatggacccccatatctttacctccaatttcaataacggcattggcaggcacaagacatacctctgttatgaggtcgagcggctcgataacggaacctcagtgaagatggaccaacacaggggtttccttcataaccaagcgaaaaatctcctttgcggtttctatgggcggcacgcggagctgagattcttggatttggtcccgagcctccagctggatcccgcgcaaatataccgcgtcacttggttcatatcatggagcccgtgctttagctgggggtgtgccggtgaggttcgcgcgttcctccaggaaaacacccatgttcgcctccggattttcgcagcgaggattttcgactatgatccactttacaaagaggccctccaaatgcttagagatgctggggctcaagtttcaattatgacatacgacgagtttaagcattgttgggacacttttgtcgatcatcagggttgtcccttccagccgtgggatggtcttgatgagcattcgcaagctttgagtggcaggcttcgcgcgatacttcagaaccagggtaactcaggttctgagacccctggcacaagtgagtcagcaacacccgagtccaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagggagacggatcaggcgggtcaacaaacctcagtgacatcatagagaaggaaactggtaagcaactggttattcaagagagcattctgatgctccctgaggaagtcgaggaagttataggaaacaagcctgagagcgatatactcgtgcatacggcgtatgacgagagcacggatgaaaatgtgatgctcttgaccagcgatgcgccagaatataaaccctgggcactggttattcaggactctaacggggaaaataaaatcaagatgctctcaggtggctccccaaagaagaaacgcaaggttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 40 - DNA, 14077 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgatattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgagaggacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattaggcctaagcggaacagcgacaagctcatcgcccgcaagaaggactgggatccgaagaagtacggcgggttcctgtggcccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctaagcagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaggctcggcgcccctagggctttcaagtacttcgacacaactatcgatccgaagcagtacaggagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaaggagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 41 - DNA, 14716 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagtcctccatcaactccggaggatctagcggaggctcctctggctctgagacacctggcacaagcgagagcgcaacacctgaaagcagcgggggcagcagcggggggtcaaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgagaggacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattaggcctaagcggaacagcgacaagctcatcgcccgcaagaaggactgggatccgaagaagtacggcgggttcctgtggcccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctaagcagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaggctcggcgcccctagggctttcaagtacttcgacacaactatcgatccgaagcagtacaggagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaaggagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 42 - DNA, 14654 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcatggaggcctctccggcttcaggtcctcgccatctcatggacccccatatctttacctccaatttcaataacggcattggcaggcacaagacatacctctgttatgaggtcgagcggctcgataacggaacctcagtgaagatggaccaacacaggggtttccttcataaccaagcgaaaaatctcctttgcggtttctatgggcggcacgcggagctgagattcttggatttggtcccgagcctccagctggatcccgcgcaaatataccgcgtcacttggttcatatcatggagcccgtgctttagctgggggtgtgccggtgaggttcgcgcgttcctccaggaaaacacccatgttcgcctccggattttcgcagcgaggattttcgactatgatccactttacaaagaggccctccaaatgcttagagatgctggggctcaagtttcaattatgacatacgacgagtttaagcattgttgggacacttttgtcgatcatcagggttgtcccttccagccgtgggatggtcttgatgagcattcgcaagctttgagtggcaggcttcgcgcgatacttcagaaccagggtaactcaggttctgagacccctggcacaagtgagtcagcaacacccgagtccatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgagaggacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatcatatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattaggcctaagcggaacagcgacaagctcatcgcccgcaagaaggactgggatccgaagaagtacggcgggttcctgtggcccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctaagcagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaggctcggcgcccctagggctttcaagtacttcgacacaactatcgatccgaagcagtacaggagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagggagacggatcaggcgggtcaacaaacctcagtgacatcatagagaaggaaactggtaagcaactggttattcaagagagcattctgatgctccctgaggaagtcgaggaagttataggaaacaagcctgagagcgatatactcgtgcatacggcgtatgacgagagcacggatgaaaatgtgatgctcttgaccagcgatgcgccagaatataaaccctgggcactggttattcaggactctaacggggaaaataaaatcaagatgctctcaggtggctccccaaagaagaaacgcaaggttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 43 - DNA, 14059 bp, Artificial Sequence
  synthetic construct
ctggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggatgatgatgataaggattacaaggatgatgatgataagatggctccaaagaagaagagaaaggttggaatccacggagttccagctgctgataagaagtactctatcggacttgccatcggaaccaactctgttggatgggctgttatcaccgatgagtacaaggttccatctaagaagttcaaggttcttggaaacaccgatagacactctatcaagaagaaccttatcggtgctcttcttttcgattctggagagaccgctgaggctaccagattgaagagaaccgctagaagaagatacaccagaagaaagaacagaatctgctaccttcaggaaatcttctctaacgagatggctaaggttgatgattctttcttccacagacttgaggagtctttccttgttgaggaggataagaagcacgagagacacccaatcttcggaaacatcgttgatgaggttgcttaccacgagaagtacccaaccatctaccaccttagaaagaagttggttgattctaccgataaggctgatcttagacttatctaccttgctcttgctcacatgatcaagttcagaggacacttccttatcgagggagaccttaacccagataactctgatgttgataagttgttcatccagcttgttcagacctacaaccagcttttcgaggagaacccaatcaacgcttctggagttgatgctaaggctatcctttctgctagactttctaagtctcgtagacttgagaaccttatcgctcagcttccaggagagaagaagaacggacttttcggaaaccttatcgctctttctcttggacttaccccaaacttcaagtctaacttcgatcttgctgaggatgctaagttgcagctttctaaggatacctacgatgatgatcttgataaccttcttgctcagatcggagatcagtacgctgatcttttccttgctgctaagaacctttctgatgctatccttctttctgacatccttagagttaacaccgagatcaccaaggctccactttctgcttctatgatcaagagatacgatgagcaccaccaggatcttacccttttgaaggctcttgttagacagcagcttccagagaagtacaaggaaatcttcttcgatcagtctaagaacggatacgctggatacatcgatggaggagcttctcaggaggagttctacaagttcatcaagccaatccttgagaagatggatggaaccgaggagcttcttgttaagttgaacagagaggatcttcttagaaagcagagaaccttcgataacggatctatcccacaccagatccaccttggagagcttcacgctatccttcgtagacaggaggatttctacccattcttgaaggataacagagagaagatcgagaagatccttaccttcagaatcccatactacgttggaccacttgctagaggaaactctcgtttcgcttggatgaccagaaagtctgaggagaccatcaccccttggaacttcgaggaggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggttgttgataagggagcttctgctcagtctttcatcgagagaatgaccaacttcgataagaaccttccaaacgagaaggttcttccaaagcactctcttctttacgagtacttcaccgtttacaacgagcttaccaaggttaagtacgttaccgagggaatgagaaagccagctttcctttctggagagcagaagaaggctatcgttgatcttcttttcaagaccaacagaaaggttaccgttaagcagttgaaggaggattacttcaagaagatcgagtgcttcgattctgttgaaatctctggagttgaggatagattcaacgcttctcttggaacctaccacgatcttttgaagatcatcaaggataaggatttccttgataacgaggagaacgaggacatccttgaggacatcgttcttacccttacccttttcgaggatagagagatgatcgaggagagactcaagacctacgctcaccttttcgatgataaggttatgaagcagttgaagagaagaagatacaccggatggggtagactttctcgtaagttgatcaacggaatcagagataagcagtctggaaagaccatccttgatttcttgaagtctgatggattcgctaacagaaacttcatgcagcttatccacgatgattctcttaccttcaaggaggacatccagaaggctcaggtttctggacagggagattctcttcacgagcacatcgctaaccttgctggatctccagctatcaagaagggaatccttcagaccgttaaggttgttgatgagcttgttaaggttatgggtagacacaagccagagaacatcgttatcgagatggctagagagaaccagaccacccagaagggacagaagaactctcgtgagagaatgaagagaatcgaggagggaatcaaggagcttggatctcaaatcttgaaggagcacccagttgagaacacccagcttcagaacgagaagttgtacctttactaccttcagaacggaagagatatgtacgttgatcaggagcttgacatcaacagactttctgattacgatgttgatgccatcgttccacagtctttcttgaaggatgattctatcgataacaaggttcttacccgttctgataagaacagaggaaagtctgataacgttccatctgaggaggttgttaagaagatgaagaactactggagacagcttcttaacgctaagttgatcacccagagaaagttcgataaccttaccaaggctgagagaggaggactttctgagcttgataaggctggattcatcaagagacagcttgttgagaccagacagatcaccaagcacgttgctcagatccttgattctcgtatgaacaccaagtacgatgagaacgataagttgatcagagaggttaaggttatcaccttgaagtctaagttggtttctgatttcagaaaggatttccagttctacaaggttagagagatcaacaactaccaccacgctcacgatgcttaccttaacgctgttgttggaaccgctcttatcaagaagtacccaaagttggagtctgagttcgtttacggagattacaaggtttacgatgttagaaagatgatcgctaagtctgagcaggagatcggaaaggctaccgctaagtacttcttctactctaacatcatgaacttcttcaagaccgagatcacccttgctaacggagagatcagaaagagaccacttatcgagaccaacggagagaccggagagatcgtttgggataagggaagagatttcgctaccgttagaaaggttctttctatgccacaggttaacatcgttaagaaaaccgaggttcagaccggaggattctctaaggagtctatccttccaaagagaaactctgataagttgatcgctagaaagaaggattgggacccaaagaagtacggaggattcgattctccaaccgttgcttactctgttcttgttgttgctaaggttgagaagggaaagtctaagaagttgaagtctgttaaggagcttcttggaatcaccatcatggagcgttcttctttcgagaagaacccaatcgatttccttgaggctaagggatacaaggaggttaagaaggatcttatcatcaagttgccaaagtactctcttttcgagcttgagaacggaagaaagagaatgcttgcttctgctggagagcttcagaagggaaacgagcttgctcttccatctaagtacgttaacttcctttaccttgcttctcactacgagaagttgaagggatctccagaggataacgagcagaagcagcttttcgttgagcagcacaagcactaccttgatgagatcatcgagcaaatctctgagttctctaagagagttatccttgctgatgctaaccttgataaggttctttctgcttacaacaagcacagagataagccaatcagagagcaggctgagaacatcatccaccttttcacccttaccaaccttggtgctccagctgctttcaagtacttcgataccaccatcgatagaaaaagatacacctctaccaaggaggttcttgatgctacccttatccaccagtctatcaccggactttacgagaccagaatcgatctttctcagcttggaggagataagagaccagctgctaccaagaaggctggacaggctaagaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggcgggggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaagacgtccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcct

SEQ ID NO: 44 - DNA, 13106 bp, Artificial Sequence
  synthetic construct
cagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggctcctaagaagaagcggaaggttggtattcacggggtgcctgcggctgccgtcaagtccatgaaggtcaagttgcgcctggataacatgccagagatcagagccggactttggaaacttcacaccgaggttaatgcgggtgtgcggtactatacggaatggcttagccttttgaggcaagaaaatctttatcggaggagtcccaatggcgatggagaacaagaatgctataaaactgctgaggaatgcaaggctgaactccttgagagactcagagcccgccaagttgagaatgggcactgcggccctgctgggagtgatgacgaactgctgcaattggcacggcaactttatgaacttctggtcccacaagcaatcggggctaaaggtgatgcgcagcaaatcgcaaggaagtttcttagtccccttgccgacaaggatgccgtgggtggtttgggaatagcaaaagcaggaaataagcctaggtgggttcggatgagggaggctggagagccaggttgggaagaggaaaaggctaaagccgaggcgagaaagagtacggatagaaccgccgatgttcttcgcgctcttgcagacttcggtcttaaacctcttatgagagtctacacagactcagacatgtccagcgtgcagtggaaaccacttcgcaaaggacaagcggtcagaacctgggatagagacatgttccaacaagcgatcgaaagaatgatgagttgggaatcgtggaatcagcgcgttggagaagcgtacgcaaagctcgtggaacaaaagtcgaggtttgaacagaaaaattttgtgggacaagaacatcttgtccaacttgtcaatcaacttcaacaagacatgaaggaagcatcacacggcctggagtcgaaagaacaaactgcgcattacttgactgggagagcgctgagagggagcgacaaagtttttgagaagtgggaaaaactcgatcctgatgccccatttgacctctatgataccgaaatcaagaatgttcaacggaggaatactcgcaggttcggatctcatgatctgtttgcgaagctcgcggaacctaaatatcaggcgctctggagagaggacgcttctttcctcacgaggtatgcggtttacaatagcattgtcagaaaactgaatcacgctaaaatgtttgcgacttttactcttccggatgctaccgcccacccgatctggacgcggtttgacaaactcggcggcaacctgcaccagtacactttcttgtttaacgaatttggcgagggcaggcacgccattcggtttcagaagctgttgacggttgaggatggcgttgctaaagaggtcgacgacgtcacggttccgatttctatgtccgcgcagctggatgacctcttgcctcgggacccacacgagctcgttgcactctacttccaggactacggtgcagaacaacatctggctggagagtttggcggcgcgaaaattcaataccgccgcgatcaattgaaccacctgcacgccagaagaggcgccagagatgtctaccttaatctgagcgtccgcgttcagtcacaatccgaagccaggggagaaaggcgccctccgtatgcagcggtcttcaggcttgttggcgataaccaccgcgcgtttgttcactttgataaattgtcagattacctcgcagaacacccagacgatggtaagctggggtcggaaggtttgctctctgggctcagagtcatgtcagttgccttgggtcttaggacttccgcgagcatatctgtcttccgcgtcgcaagaaaggacgaattgaagccgaacagtgaaggccgggtccctttttgcttcccgatcgaagggaacgaaaacctcgttgctgtccacgagcggagccaactgttgaagcttcccggtgaaacggaatcgaaagatctgagagcgatcagagaagagcgccaaaggacgcttagacagctccggacgcaacttgcatacttgcgccttctggttcgctgcggtagtgaagacgttggaagaagagagaggtcatgggctaaactcatagagcaacctatggatgctaatcaaatgacgcctgattggagagaagcattcgaagacgaacttcagaaactgaaatccctttacgggatatgcggcgatcgcgagtggacagaagcagtgtatgagtctgtgaggcgcgtgtggcggcatatgggtaaacaggtgcgcgattggagaaaagacgttaggagcggggaaagacctaagatacggggatatcagaaagacgttgtcgggggaaatagcattgaacagattgaatatttggagcgccaatataagttcctcaaatcctggtctttcttcggcaaagtgtcaggccaggtgatacgcgcggaaaagggatcgcgctttgcaataactctgagagaacatattgatcatgccaaagaagatcggttgaagaaactcgccgatagaatcatcatggaggcgcttggttatgtctacgccttggacgatgaacggggaaagggaaagtgggtcgccaagtatccaccttgccaactcattctcctcgaagaactttccgaataccagtttaacaacgatcggccgccatcagagaataatcaactgatgcagtggtcccatcgcggtgtgtttcaagagttgctcaatcaggcccaagtccatgatctgcttgttggcacaatgtatgcagccttttcctcccggtttgatgcaagaacaggggctcctggcatacgctgtagacgggtcccggcgaggtgcgcccgcgaacaaaaccctgaaccgttcccctggtggttgaacaagttcgttgcggagcacaagctggacgggtgtcctctgcgggccgacgatcttattcccaccggggaaggggaattctttgtgagccctttctcggcggaggaaggggattttcaccaaatacatgcagatcttaatgccgcacaaaatttgcagaggagactgtggtcagactttgatattagtcagatacgcctccgctgtgactggggagaggtcgatggcgagcctgtgttgataccaagaacgaccggaaagaggacagccgattcgtatggaaacaaggttttttacacgaagacgggcgttacttactacgaaagagaaagagggaagaagagaaggaaagtctttgcccaagaagaattgagcgaggaagaagccgagctcttggtcgaagcggacgaggcacgggaaaagtctgtcgtcctcatgagggacccttccggaattattaaccggggagattggacgcggcagaaagagttttggtccatggttaatcaacgcatagaaggctaccttgtcaagcaaataagaagtcgcgtgagattgcaggagagtgcatgtgagaacactggggacataaagcgtcctgctgccaccaaaaaggccggacaggctaagaaaaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggaggcggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaacgacgtccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacagcttccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctctggaccagccagga

SEQ ID NO: 45 - DNA, 14254 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgctattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatgctatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattctgcctaagcggaacagcgacaagctcatcgcccgcaagaaggactgggatccgaagaagtacggcgggttcgacagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctggcgagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctgctgctttcaagtacttcgacacaactatcgatcgcaagaggtacacaagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggaggcggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 46 - DNA, 14254 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgccattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgaggctacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatgctatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattcgccctaagcggaacagcgacaagctcatcgcccgcaagaaggactgggatccgaagaagtacggcgggttcgttagccccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctcgcttcctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaacctcggcgcccctcgcgctttcaagtacttcgacacaactatcgatcgcaaggtctaccgcagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggaggcggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 47 - DNA, 14254 bp, Artificial Sequence
  synthetic construct
tggaccagccaggacagaaatgcctcgacttcgctgctacccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacattatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgcgagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgcttgcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgcccttcaccatggattacaaggaccacgacggggattacaaggaccacgacattgattacaaggatgatgatgacaagatggctccgaagaagaagaggaaggttggcatccacggggtgccagctgctgacaagaagtactcgatcggcctcgccattgggactaactctgttggctgggccgtgatcaccgacgagtacaaggtgccctcaaagaagttcaaggtcctgggcaacaccgatcggcattccatcaagaagaatctcattggcgctctcctgttcgacagcggcgagacggctgagaggacgcggctcaagcgcaccgcccgcaggcggtacacgcgcaggaagaatcgcatctgctacctgcaggagattttctccaacgagatggcgaaggttgacgattctttcttccacaggctggaggagtcattcctcgtggaggaggataagaagcacgagcggcatccaatcttcggcaacattgtcgacgaggttgcctaccacgagaagtaccctacgatctaccatctgcggaagaagctcgtggactccacagataaggcggacctccgcctgatctacctcgctctggcccacatgattaagttcaggggccatttcctgatcgagggggatctcaacccggacaatagcgatgttgacaagctgttcatccagctcgtgcagacgtacaaccagctcttcgaggagaaccccattaatgcgtcaggcgtcgacgcgaaggctatcctgtccgctaggctctcgaagtctcggcgcctcgagaacctgatcgcccagctgccgggcgagaagaagaacggcctgttcgggaatctcattgcgctcagcctggggctcacgcccaacttcaagtcgaatttcgatctcgctgaggacgccaagctgcagctctccaaggacacatacgacgatgacctggataacctcctggcccagatcggcgatcagtacgcggacctgttcctcgctgccaagaatctgtcggacgccatcctcctgtctgatattctcagggtgaacaccgagattacgaaggctccgctctcagcctccatgatcaagcgctacgacgagcaccatcaggatctgaccctcctgaaggcgctggtcaggcagcagctccccgagaagtacaaggagatcttcttcgatcagtcgaagaacggctacgctgggtacattgacggcggggcctctcaggaggagttctacaagttcatcaagccgattctggagaagatggacggcacggaggagctgctggtgaagctcaatcgcgaggacctcctgaggaagcagcggacattcgataacggcagcatcccacaccagattcatctcggggagctgcacgctatcctgaggaggcaggaggacttctaccctttcctcaaggataaccgcgagaagatcgagaagattctgactttcaggatcccgtactacgtcggcccactcgctaggggcaactcccgcttcgcttggatgacccgcaagtcagaggagacgatcacgccgtggaacttcgaggaggtggtcgacaagggcgctagcgctcagtcgttcatcgagaggatgacgaatttcgacaagaacctgccaaatgagaaggtgctccctaagcactcgctcctgtacgagtacttcacagtctacaacgagctgactaaggtgaagtatgtgaccgagggcatgaggaagccggctttcctgtctggggagcagaagaaggccatcgtggacctcctgttcaagaccaaccggaaggtcacggttaagcagctcaaggaggactacttcaagaagattgagtgcttcgattcggtcgagatctctggcgttgaggaccgcttcaacgcctccctggggacctaccacgatctcctgaagatcattaaggataaggacttcctggacaacgaggagaatgaggatatcctcgaggacattgtgctgacactcactctgttcgaggaccgggagatgatcgaggagcgcctgaagacttacgcccatctcttcgatgacaaggtcatgaagcagctcaagaggaggaggtacaccggctgggggaggctgagcaggaagctcatcaacggcattcgggacaagcagtccgggaagacgatcctcgacttcctgaagagcgatggcttcgcgaaccgcaatttcatgcagctgattcacgatgacagcctcacattcaaggaggatatccagaaggctcaggtgagcggccagggggactcgctgcacgagcatatcgcgaacctcgctggctcgccagctatcaagaaggggattctgcagaccgtgaaggttgtggacgagctggtgaaggtcatgggcaggcacaagcctgagaacatcgtcattgagatggcccgggagaatcagaccacgcagaagggccagaagaactcacgcgagaggatgaagaggatcgaggagggcattaaggagctggggtcccagatcctcaaggagcacccggtggagaacacgcagctgcagaatgagaagctctacctgtactacctccagaatggccgcgatatgtatgtggaccaggagctggatattaacaggctcagcgattacgacgtcgatgctatcgttccacagtcattcctgaaggatgactccattgacaacaaggtcctcaccaggtcggacaagaaccggggcaagtctgataatgttccttcagaggaggtcgttaagaagatgaagaactactggcgccagctcctgaatgccaagctgatcacgcagcggaagttcgataacctcacaaaggctgagaggggcgggctctctgagctggacaaggcgggcttcatcaagaggcagctggtcgagacacggcagatcactaagcacgttgcgcagattctcgactcacggatgaacactaagtacgatgagaatgacaagctgatccgcgaggtgaaggtcatcaccctgaagtcaaagctcgtctccgacttcaggaaggatttccagttctacaaggttcgggagatcaacaattaccaccatgcccatgacgcgtacctgaacgcggtggtcggcacagctctgatcaagaagtacccaaagctcgagagcgagttcgtgtacggggactacaaggtttacgatgtgaggaagatgatcgccaagtcggagcaggagattggcaaggctaccgccaagtacttcttctactctaacattatgaatttcttcaagacagagatcactctggccaatggcgagatccggaagcgccccctcatcgagacgaacggcgagacgggggagatcgtgtgggacaagggcagggatttcgcgaccgtcaggaaggttctctccatgccacaagtgaatatcgtcaagaagacagaggtccagactggcgggttctctaaggagtcaattaggcctaagcggaacagcgacaagctcatcgcccgcaagaaggactgggatccgaagaagtacggcgggttcctgtggcccactgtggcctactcggtcctggttgtggcgaaggttgagaagggcaagtccaagaagctcaagagcgtgaaggagctgctggggatcacgattatggagcgctccagcttcgagaagaacccgatcgatttcctggaggcgaagggctacaaggaggtgaagaaggacctgatcattaagctccccaagtactcactcttcgagctggagaacggcaggaagcggatgctggcttccgctaagcagctgcagaaggggaacgagctggctctgccgtccaagtatgtgaacttcctctacctggcctcccactacgagaagctcaagggcagccccgaggacaacgagcagaagcagctgttcgtcgagcagcacaagcattacctcgacgagatcattgagcagatttccgagttctccaagcgcgtgatcctggccgacgcgaatctggataaggtcctctccgcgtacaacaagcaccgcgacaagccaatcagggagcaggctgagaatatcattcatctcttcaccctgacgaggctcggcgcccctagggctttcaagtacttcgacacaactatcgatccgaagcagtacaggagcactaaggaggtcctggacgcgaccctcatccaccagtcgattaccggcctctacgagacgcgcatcgacctgtctcagctcgggggcgacaagcggccagcggcgacgaagaaggcggggcaggcgaagaagaagaagaagggagacggctctggatcggggtcgggttctggctcagtcgacgatgctcttgacgattttgacctcgatatgctcgacgctcttgatgattttgatctcgacatgctcgatgcacttgatgactttgaccttgacatgctcgacgcactcgatgacttcgacctcgacatgcttgagggcagaggaagtcttctaacatgcggtgacgtggaggagaatcccggccctatggcgtcaaatttcacgcagtttgttttggttgataacggcgggactggcgacgttacagtagctccatcaaattttgcgaacggagtcgctgagtggattagctcaaattcaaggtcccaggcctacaaggttacctgttctgttaggcagagttctgcgcaaaaaagaaaatataccatcaaggttgaagtccctaaagttgcaacacaaacagtcggtggtgttgagctccctgtggcagcctggagatcttacttaaacatggagctaacaattccaatattcgctacaaactctgattgtgaactgattgttaaggcgatgcaaggtctcttgaaagatggaaaccctataccgtccgctatcgcagctaacagcggtatctatcctaagaagaagaggaaggttggctctggatcggggtcgggttctggctcaggatccggtacctatccctatgacgtgcccgattatgccagcctgggcagcggctcccccaagaaaaaacgcaaggtggaagatcctaagaaaaagcggaaagtggacggcattggtagtgggagcaacggcagcagcggatccaacggtccgactgacgccgcggaagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaggaattgctatcgaaaaattatcatcttgagaacgaagttgctaggctcaaaaagggctcaggctcaggcaccgcggtaaacataggtggtggaaccggtccgatggatctacagcggccgcaaggtggaggtggacccaagaagaagcgcaaggtgtgaactagttaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgcccgtacgctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggcgccatggggccggacatagtcatgacacaaagtccgagttcgctttctgcgagtgttggggatagagttacgataacctgcagatctagtacgggggccgtcaccacgtcgaattacgcgtcatgggtccaagaaaagccaggcaaactgtttaagggtcttataggtggcacgaacaaccgggctcccggagtcccttccaggtttagtggatcacttattggtgacaaggcaacgctgacgatatcttccctccagccagaagatttcgcgacctacttttgtgcactttggtattctaaccactgggtctttggccaagggacgaaggtcgagctcaaacggggtggcggtgggagcggcgggggaggatcagggggcggaggttcgagtggcggggggagcgaagtcaaactcctcgagtccggtggaggattggtgcaaccaggtggttcattgaagctctcgtgtgcagtttctggtttctctttgacagactacggtgtcaattgggtccggcaggcaccggggcgcggtcttgagtggatcggcgttatctggggcgacggaataacagattataactctgctttgaaggacagattcatcattagcaaggacaacgggaagaacacggtgtacctccagatgagcaaagtgcgcagtgatgatactgccctgtattactgcgtcacaggtttgtttgattactggggtcaaggcactcttgtgacagtctcttcgtatccttatgacgtgcccgactacgctggcggagggggcggcagcggaggcggtggatccggaggcggtggctcagggggcggcgggtcgcttgatcctggtggcggaggttctgggagcaaaggagaggagctctttacaggtgtcgtccctattttggtggagcttgatggagatgtcaatggccataaattttccgtcaggggcgaaggggagggggacgcgactaatggaaaattgaccctcaagtttatctgcacgacaggaaagttgccggtcccctggcccactctggttaccaccctcacttatggggtccagtgctttagcagataccctgatcatatgaaacgccatgacttcttcaaatcagccatgccagagggttatgtgcaggaaagaactataagctttaaggacgatgggacgtacaaaacgcgggcagaggttaagtttgagggggatactcttgtgaataggatcgaacttaagggcatcgattttaaagaagacggtaatattcttggccacaagttggagtataatttcaattctcacaacgtgtacataacagcagataaacagaaaaatgggatcaaggccaattttaaaattaggcataatgttgaagatgggtctgtgcaactggcagatcattatcagcagaatactccaattggcgatggccctgtcttgttgcctgacaatcattacctttccacgcagtcagtcctgtccaaagatccgaatgagaagcgggaccacatggtcttgctcgaattcgttactgccgccggaatcactcatggcatggacgagctttacaagggaggtggaaggactggaggtggcggaggggggctgcttgaccccggaacccctatggatgccgacttggtggcctcctccacggttgtttgggaacaggatgccgacccttttgcgggcactgctgacgattttcccgctttcaatgaggaggagttggcttggctgatggaacttctcccacagggtggtagtggtggcctcctggatccggggacaccgatggacgcagacctggtggcgtcgtcaacggttgtgtgggaacaggatgctgatccgttcgcaggaactgccgacgattttcctgcattcaacgaagaggaactcgcatggctcatggagcttctgcctcagggatcgggtggcgggtctcgaacggaagaatacaagcttatattgaacggtaagacactgaaaggtgaaactacgacagaagccgttgacgcggcaactgcggagaaagtctttaagcagtatgctaacgataatggcgtggacggggagtggacgtatgacgacgcaaccaaaacattcacggtcaccgaggggggcggcagcggaggcggtacctcgcccaaaacccggagacgccctaggcgctcgcagcgcaaaagacctcccacgtaaagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgttccggaactctagataagcttaccggaaagggcgaattcgcaactttgtatacaaaagttgaacgagaaacgtaaaatgatataaatatcaatatattaaattagattttgcataaaaaacagactacataatactgtaaaacacaacatatccagtcactatgccatccagctgatatcccctatagtgagtcgtattacatggtcatagctgtttcctggcagctctggcccgtgtctcaaaatctctgatgttacattgcacaagataaaaatatatcatcatgcctcctc

SEQ ID NO: 48 - DNA, 240 bp, Oryza sativa
cgagagtacattcccacggaggaatgaaaatccatctccgaagaaaagctttagctttggtagagcgagcgaaagctgcattggccacgcgagccaactaacccctccgagtccaggccggttggtacacgtgtcgccgccgccgtccgtttcccacccggagcccacgtggccgccatccgcccgtccgcccgacctaaccaccccccctccccctcgcgcctatatcagcacgcgccc

SEQ ID NO: 49 - DNA, 240 bp, Oryza sativa
gtagttggaacgtgccactgcccactgcacgactttagttcttcgaccctgtgtgagtagtagcgaacccagcctctctatgtagcgtgctccacccgtacagtacgcccctccctatatacggatgatcacgcagtacacgctctccctatcgtaaaacccgcgaaaaccacaaccgaaacgcaagggtcgatccatccacgcctatcgcgcgcgcgtgcatcgggtcgatcgatcgag

SEQ ID NO: 50 - DNA, 240 bp, Oryza sativa
gtagttggaacgtgccactgcccactgcacgactttagttcttcgaccctgagtgagtagtagcgaacccagcctctctatgtagcgtgctccacccgtacagtacgcccctccctatatacggatgatcacgcagtacacgctctccctatcgtaaaacccgcgaaaaccacaaccgaaacgcaagggtcgatccatccacgcctatcgcgcgcgcgtgcatcgggtcgatcgatcgag

SEQ ID NO: 51 - DNA, 240 bp, Oryza sativa
tatgaaaaaaaattaaaaaaacaagtcacacataaaatattaatcatgttttatcatctaacaataatgaaaatacgaattataaaaaaatttcatataagacggacagtcaaagttggacacggaaacctagagtaacttgttaggcagtacaagtgtgtgtagctatactccccctgtcctgtaccagcttatatatataggcgagccaacgagcgagagccatcaccaagtgcaagg

SEQ ID NO: 52 - DNA, 240 bp, Oryza sativa
gtgcaagaaggcttgcaggccagagatagagacaagacatccccgttcaccagcagcacaaaagaacgcagcaacgatcagtggcagcgttgcaattacggaccggtgtggcggtgttgttgcccatcccatcaatttagcagctgcgatgtcaccaccacaactattcattatagccacccagctaacaccatctcgtgcgctcgtggcccttggattcaactacccccgcgccttgat

SEQ ID NO: 53 - DNA, 240 bp, Oryza sativa
tctatgatatgtgccaagtagtcgagagccaaataaaataaaattattagtgattgcaacaggtgggctggttttgcgtcatttgaccagtacacagccacacatgccatgaggagaaaaggaatcaagcaaacaacctttggtacctgttcggtggtaggtacgaagggtagtttgttagctgcacagcacgcgggggtaccgtgtatatatcaactacccagtccgcgttttcctctc

SEQ ID NO: 54 - DNA, 240 bp, Oryza sativa
actcttgagttttggccgtctatcacccgtacgtcgcacgaaggcggccatgtgtttacctactgttctctcctgcctccacactttctggcacggtacaaaagccatacatagccacactgccacactcattgcgtgtgtgtcgaagacggccgggcgctgctagatctaccaagcactcgatcagctttcagctctgggtgttagccagcaagaacccatcggtcgatcgatcgatcc

SEQ ID NO: 55 - DNA, 240 bp, Oryza sativa
aaaacgacatgtatgaaccgtttttttttcctttgtcgacacatgtgcatagtagcccctatagtccacgtgaccgacctcggcatgagccgtcgggtgcagccaccagtcaaaccccaccaccttcaaatcaattcttccatccccaactatcagaccatctccctacctaagctccctcactcactcgcctttacttatcgatctcgatcatccacgagctagctagcagtgctcgcc

SEQ ID NO: 56 - DNA, 260 bp, Oryza sativa
aaaacgacatgtatgaaccgttttttttttcctttgtcgacacatgtggaggccggggagtagtacgcatagtagctcctatagtccacgtgaccgacctcggcatgagccgtcgtgtgcagccaccagtcaaaccccaccaccttcaaatcaattcttccatccccaactatcagaccatctccctacctaagctccctcactcactcgcctttacttatcgatctcgatcatccacgagctagctagcagtgctcgcc

SEQ ID NO: 57 - DNA, 240 bp, Oryza sativa
gaggactgacctgtcctgccggttgctcaagaactcatgagaggctggttgagtaaatgagtacatcctgtttaagttaaactgattccagtcgaaagtcgaaaccagctttgactataagtcactgacggtctggccccaccgttgaatcagcttcaggcggccaccaactgcagcagcccggcggccgcatgcccgcatcgtcgcccgttcaaccctcgtgccgagctgacgtttacc

SEQ ID NO: 58 - DNA, 240 bp, Oryza sativa
aaagtctgtcgcctgacaccgacacttatactgtagatccgcccctaaccacggcaccaatgattgtatctggttagtggagtaattgagacgaccaaatccctccccttgagataaccacatcatctaagaacggaatgccttgtgcaaattggagtcactacatatgacatagccagacgtgtctcctgctctctgcttgagatcaacaagagagagagagagagagagagcaagaga

SEQ ID NO: 59 - DNA, 122 bp, Artificial Sequence
  synthetic construct
ggtctagaggacagaatttttcaacgggtgtgccaatggccactttccaggtggcaaagcccgttgagcttctcaggccaacatgaggatcacccatgtctgcagggcctgagaagtggcac

SEQ ID NO: 60 - DNA, 156 bp, Artificial Sequence
  synthetic construct
ggccgacgtctgtacccactaggagtacaaggccggtctagaggacagaatttttcaacgggtgtgccaatggccactttccaggtggcaaagcccgttgagcttctcaggccaacatgaggatcacccatgtctgcagggcctgagaagtggcac

SEQ ID NO: 61 - DNA, 115 bp, Artificial Sequence
  synthetic construct
gtctaaaggacagaatttttcaacgggtgtgccaatggccactttccaggtggcaaagcccgttgaacttcggccgacgtctgtacccactaggagtacaaggccgaagtggcac

SEQ ID NO: 62 - DNA, 149 bp, Artificial Sequence
  synthetic construct
ggccaacatgaggatcacccatgtctgcagggccgtctaaaggacagaatttttcaacgggtgtgccaatggccactttccaggtggcaaagcccgttgaacttcggccgacgtctgtacccactaggagtacaaggccgaagtggcac

SEQ ID NO: 63 - DNA, 29 bp, Oryza sativa
catgtgtaatctggagagctcaccggaga

SEQ ID NO: 64 - DNA, 29 bp, Solanum lycopersicum
cttcagcctcctaagcttaaactaggtga

SEQ ID NO: 65 - DNA, 29 bp, Oryza sativa
gtaatctggagagctcaccggagagctgg

SEQ ID NO: 66 - DNA, 40 bp, Arabidopsis thaliana
taaccggtggtgaacataggctgaggaattataaatcggt

SEQ ID NO: 67 - DNA, 20 bp, Artificial Sequence
  synthetic construct
taaccggtggtgaacatagg

SEQ ID NO: 68 - DNA, 41 bp, Artificial Sequence
  synthetic construct
taaccggtggtgaacataggactgaggaattataaatcggt

SEQ ID NO: 69 - DNA, 41 bp, Artificial Sequence
  synthetic construct
taaccggtggtgaacataggtctgaggaattataaatcggt

SEQ ID NO: 70 - DNA, 41 bp, Artificial Sequence
  synthetic construct
taaccggtggtgaacatagggctgaggaattataaatcggt

SEQ ID NO: 71 - DNA, 23 bp, Arabidopsis thaliana
caagtccctcgtcgtatgattgg

SEQ ID NO: 72 - DNA, 23 bp, Arabidopsis thaliana
gaccaaccgatcatcttgaccgg

SEQ ID NO: 73 - DNA, 18 bp, Arabidopsis thaliana
tgcattaactcgggtcgg

SEQ ID NO: 74 - DNA, 23 bp, Populus trichocarpa
tcctcaggctagacttggtcagg

SEQ ID NO: 75 - DNA, 24 bp, Artificial Sequence
  synthetic construct
tcctcaggctagacttgagtcagg

SEQ ID NO: 76 - DNA, 22 bp, Artificial Sequence
  synthetic construct
tcctcaggctagacttgtcagg

SEQ ID NO: 77 - DNA, 24 bp, Artificial Sequence
  synthetic construct
tcctcaggctagacttgtgtcagg

SEQ ID NO: 78 - DNA, 24 bp, Artificial Sequence
  synthetic construct
tcctcaggctagacttgcgtcagg

SEQ ID NO: 79 - DNA, 17 bp, Artificial Sequence
  synthetic construct
tcctcaggctagactgg

(19) United States
(12) Patent Application Publication
(10) Pub. No.: US 2023/0374528 A1
(43) Pub. Date: Nov. 23, 2023

(54) COMPOSITIONS, SYSTEMS, AND METHODS FOR ORTHOGONAL GENOME ENGINEERING IN PLANTS

(71) Applicant: UNIVERSITY OF MARYLAND, COLLEGE PARK, College Park, MD (US)

(72) Inventors: YIPING QI, Potomoc, MD (US); CHANGTIAN PAN, College Park, MD (US)

(21) Appl. No.: 18/040,942

(22) PCT Filed: Aug. 17, 2021
(86) PCT No.: PCT/US2021/046281

§ 371 Date: Feb. 7, 2023

Related U.S. Application Data
(60) Provisional application No. 63/066,674, filed on Aug. 17, 2020.

(51) Int. Cl. C12N 15/82; C12N 9/22; C12N 15/11; C12N 15/115

(52) U.S. Cl. CPC C12N 15/8213; C12N 9/22; C12N 15/11; C12N 15/115; C12N 2310/20; C12N 2310/16; C12N 2310/3519; C07K 2319/00

(57) ABSTRACT

The present disclosure relates to compositions and methods for simultaneous and combinational CRISPR-mediated gene activation, gene editing, and gene repression in plants based on a single Cas protein.

### CROSS-REFERENCE TO RELATED APPLICATION

This application claims priority to provisional application U.S. Ser. No. 63/066,674, filed Aug. 17, 2020, which is hereby incorporated herein by reference in its entirety.

### GOVERNMENT SUPPORT

This invention was made with government support under IOS1758745 and IOS2029889 awarded by the National Science Foundation. The government has certain rights in the invention.

### SEQUENCE LISTING

The instant application contains a sequence listing, which has been submitted in ASCII format by electronic submission and is hereby incorporated by reference in its entirety. Said ASCII copy, created on Aug. 16, 2021, is named P13668WO00_ST25.txt and is 297,208 bytes in size.

### TECHNICAL FIELD

The present disclosure relates to compositions and methods for editing genomic sequences and for modulating gene expression in plants.

### BACKGROUND

CRISPR-Cas9, since its first demonstration as RNA-guided nuclease, has been rapidly applied for genome editing in eukaryotes including plants. Predominant use of CRISPR-Cas9 has been based on targeted mutagenesis through error-prone non-homologous end joining (NHEJ) repair of Cas9-induced DNA double-strand breaks (DSBs). In recent years, Cas9-derived base editors such as cytosine base editors (CBEs) and adenine base editors (ABEs) have gained momentum on conferring precise base changes in genomes of interest. Dual base editors that confer simultaneous C-to-T and A-to-G base edits have also been developed, including synchronous programmable adenine and cytosine editor (SPACE), A&C-BEmax, and Target-ACEmax demonstrated in human cells, and saturated targeted endogenous mutagenesis editors (STEMEs) demonstrated in plants. Furthermore, a SWISS platform was developed for simultaneous adenine base editing, cytosine base editing, and indel formation in plant genomes. These multifunctional CRISPR systems however limit their capabilities to solely genome editing.

Aside from genome editing, the CRISPR-Cas9 system has been repurposed for genome reprogramming. CRISPR activation (CRISPRa) systems allow for transcriptional activation, and such systems were developed in mammalian cells and plant cells. On the contrary, CRISPR interference (CRISPRi) systems were used for transcriptional repression in mammalian cells and plants. These transcription regulation systems are based on deactivated Cas9 (dCas9) which abolishes nuclease activity while retaining single guide RNA (sgRNA)-mediated DNA binding activity. Coupled with engineered sgRNA scaffolds for the recruitment of activators and repressors, CRISPR-dCas9 was demonstrated for simultaneous transcriptional activation and repression in human cells and yeast. Alternatively, the DNA cleavage activity of Cas9 can be abolished without compromise on DNA binding by using truncated protospacers. Based on a similar principle, a nuclease active AsCas12a-VPR fusion was engineered for orthogonal genome editing and transcriptional activation in mice. However, direct fusion of a transcriptional activator to a Cas protein would prevent its use for transcriptional repression. On the other hand, nuclease active Cas12a was also used to develop a dual functional CRISPR system for simultaneous gene editing and repression in Corynebacterium glutamicum. Orthogonal genome editing and transcriptional activation could also be achieved by using orthogonal Cas9 proteins. However, programming functionalities through guide RNAs appears to be more versatile as well as easier for vector construction and delivery. Further, a robust CRISPR system for simultaneous genome editing, transcriptional activation, and repression is yet to be developed in any organism. One constraint that has limited orthogonal CRISPR applications in plants is the lack of a highly efficient CRISPRa system.

It is an objective of the present disclosure to provide an improved CRISPRa system with higher levels of gene activation. It is a further objective of the present disclosure to provide an orthogonal CRISPR system for simultaneous genome editing, gene activation, and gene repression. Additional objectives, features, and advantages will become apparent based on the disclosure contained herein.

### SUMMARY

The presently disclosed subject matter relates generally to genome engineering. A potent CRISPR transcriptional activation system in plants, termed CRISPR-Act3.0, is provided. The system provides higher levels of gene activation than all other gene activation systems reported in plants to date. Further provided is a comprehensive platform called CRISPR-Combo, which allows for simultaneous and combinational gene activation, editing, and repression. The platform enables multiple genome engineering outcomes in plants including potent single or multi-gene activation; simultaneous gene editing and gene activation; simultaneous gene editing and gene repression; simultaneous gene activation and repression; and simultaneous gene editing, activation, and repression. The gene editing may include non-homologous end joining (NHEJ) based mutagenesis, base editing, prime editing, and homology-based repair (HDR).

Systems for activating expression of a target nucleic acid are provided, the systems comprising (i) a Cas polypeptide, or a polynucleotide encoding the Cas polypeptide; (ii) a guide polynucleotide comprising an aptamer; (iii) a polypeptide comprising an adapter domain and a multimerized epitope, wherein the adapter domain binds the aptamer, or a polynucleotide encoding the polypeptide; and (iv) a polypeptide comprising an affinity domain and a transcriptional activation domain, wherein the affinity domain binds the multimerized epitope, or a polynucleotide encoding the polypeptide.

Systems for simultaneously activating expression of a target nucleic acid and modifying a nucleotide sequence at a target site in a genome are provided, the systems comprising: (i) a Cas polypeptide, or a polynucleotide encoding the Cas polypeptide; (ii) a dead guide polynucleotide that mediates increased expression of a target nucleic acid, wherein the dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a guide polynucleotide that mediates sequence-specific cleavage at a target site in the genome.

Systems for simultaneously activating expression of a first target nucleic acid and repressing expression of a second target nucleic acid are provided, the systems comprising (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a second dead guide polynucleotide that mediates reduced expression of the second target nucleic acid.

Systems for simultaneously activating expression of a first target nucleic acid, repressing expression of a second target nucleic acid, and modifying a nucleotide sequence at a target site in a genome are provided, the systems comprising (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; (v) a second dead guide polynucleotide that mediates reduced expression of the second target nucleic acid; and (vi) a guide polynucleotide that mediates sequence-specific cleavage at a target site in the genome.

Methods to use these systems to activate the expression of a target nucleic acid in a plant cell, repress the expression of a target nucleic acid in a plant cell, and/or modify a nucleotide sequence at a target site in a genome of a plant cell are described herein. Modified plants and plant cells are also encompassed.

While multiple example embodiments are disclosed, still other example embodiments of the inventions will become apparent to those skilled in the art from the following detailed description, which shows and describes illustrative example embodiments of the invention. Accordingly, the figures and detailed description are to be regarded as illustrative and not restrictive in any way.

### BRIEF DESCRIPTION OF THE FIGURES

The following drawings form part of the specification and are included to further demonstrate certain example embodiments or various aspects of the invention. In some instances, example embodiments can be best understood by referring to the accompanying figures in combination with the detailed description presented herein. The description and accompanying figures may highlight a certain specific example, or a certain aspect of the invention. However, one skilled in the art will understand that portions of the examples or aspects provided in the present disclosure may be used in combination with other examples or aspects of the invention.

FIG. 1A-D shows recruiting VP64 with different sgRNA scaffolds for gene activation in rice protoplasts. FIG. 1A shows diagrams of gR2.0, gR8xMS2 and gR16xMS2 scaffolds, with gR2.0 containing two MS2 RNA aptamers, gR8xMS2 containing eight unique MS2 RNA aptamers, and gR16xMS2 containing 16 MS2 RNA aptamers as 14 tandem repeats of MS2 are fused to the 3′ end of the gR2.0 sequence. FIG. 1B-D shows a comparison of gR2.0, gR8xMS2 and gR16xMS2-mediated OsGW7 (FIG. 1B) and OsER1 (FIG. 1C) activation in rice cells. Both RNA polymerase Pol III promoter OsU3 and RNA polymerase Pol II promoter ZmUbi were tested for expressing each sgRNA. To guide proper sgRNA maturation in the ZmUbi promoter system, a tRNA-gR2.0 expression system was employed. FIG. 1D shows qRT-PCR analysis of sgRNA expression levels of gR2.0, gR8xMS2 and gR16xMS2 scaffolds. For qRT-PCR assays (FIG. 1B-D), T-DNA vectors without sgRNAs served as the negative control (CTRL). OsTubulin is used as the endogenous control gene. All data are presented as the mean ±s.d. (n=2 independent experiments for FIG. 1B-C, n=3 independent experiments for FIG. 1D).

FIG. 2A-C shows testing of a split GFP-based activator recruitment system in rice protoplasts. FIG. 2A shows a schematic illustration of the split-GFP activation system, which consists of dCas9- GFP11x7, MCP-GFP11x7, GFP1-10-VP64. MCP, MS2 bacteriophage coat protein. GFP11x7, seven copies of GFP11 peptide. GFP1-10, the remaining split-GFP fragment. Neither GFP1-10 nor GFP11 fluoresce independently but a strong signal is restored when they reconstitute functional GFP proteins. Note three sgRNA scaffolds (gR2.0, gRx8MS2 and gRx16MS2) were compared with this activation strategy. Both RNA polymerase Pol III promoter OsU3 and RNA polymerase Pol II promoter ZmUbi were tested for expressing each sgRNA. To guide proper sgRNA maturation in the ZmUbi promoter system, a tRNA-gR2.0 expression system was employed. FIG. 2B-C shows qRT-PCR analysis of the split GFP-based OsGW7 (FIG. 2B) and OsER1 (FIG. 2C) activation coupled with different activator recruitment systems. For qRT-PCR assays (FIG. 2B-C), T-DNA vectors without sgRNAs served as the negative control (CTRL). OsTubulin is used as the endogenous control gene. All data are presented as the mean ±s.d. (n=2 independent experiments).

FIG. 3A-G shows development of the CRISPR-Act3.0 system. FIG. 3A is a schematic illustration of the CRISPR-Act3.0 strategy. The dSpCas9 is fused with a VP64, and the coupled gR2.0 contains two MS2 RNA aptamers for recruiting the MS2 bacteriophage coat protein (MCP), which is fused to the SunTag. The single-chain variable fragment (scFv) of GCN4 antibody fused to a super folder green fluorescent protein (sfGFP), which serves as a linker for the scFv and activator fusion. PAM, protospacer adjacent motif. TSS, transcriptional start site. FIG. 3B shows a comparison of different sgRNA scaffolds and 4x or 10xGCN4 epitopes for gene activation. Act2.0, a 2^{nd} generation of CRISPR-activation system, CRISPR-Act2.0. 4x or 10x GCN4, four or ten repeats of GCN4 epitopes. gR8xMS2, the sgRNA containing eight unique MS2 RNA aptamers. FIG. 3C shows a comparison of different activators for gene activation. 2xTAD, two repeats of TAD (TAL Activation Domain). 2xTAD-VP64, two repeats of TAD coupled with a VP64. TV, six copies of the TALE TAD motif coupled with VP128 (6xTAL-VP128). VPR, VP64-p65-Rta. Different letters indicate significantly different mean values at p<0.05 (one-way ANOVA with post-hoc Tukey test). FIG. 3D shows a comparison between the CRISPR-Act3.0 and three other potent 2^{nd} generation CRISPR-activation systems. Act3.0-ZmUbi, a Pol II promoter, ZmUbi, was used for sgRNA expression, coupled with the tRNA processing system. The other systems used OsU3 for sgRNA expression. FIG. 3E shows activation of an mCherry gene by an sgRNA. Tested promoters with intact 5′ UTR sequences are fused to the mCherry coding sequence. FIG. 3F shows detection of mCherry signals without (−Act3.0) and with the CRISPR-Act3.0 activation system (+Act3.0) in rice cells. mCherry signals were detected using a fluorescence microscope 24 hours after rice protoplast transformation. FIG. 3G shows statistical analysis of mCherry positive cells with and without CRISPR-Act3.0 activation system. All data are presented as the mean ±s.d. (n=5 independent scopes). **p<0.01, two-tailed Student's t-tests. For quantitative reverse transcription PCR (qRT-PCR) assays (FIG. 3B-D), T-DNA vectors without sgRNAs served as the negative control (CTRL). OsTubulin is used as the endogenous control gene. All data are presented as the mean ±s.d. (n=3 independent experiments). **p<0.01, two-tailed Student's t-tests.

FIG. 4A-D shows determination of the CRISPR-Act3.0 system-induced activation efficiency in rice protoplasts. FIG. 4A is a diagram of the target site positions of OsBBM1 for activation. The sgRNAs shown above target the coding strand. The sgRNAs shown below target the noncoding strand. The gR1 to 3 represent the sgRNA1 to 3, respectively. TSS, transcription start site. The numbers adjacent to sgRNAs indicate that the distance from sgRNA targetsites (3′) to TSS (bp). FIG. 4B shows a comparison of the activation efficiency between CRISPR-Act3.0 and three potent 2^{nd} generationCRISPR-activation systems with different sgRNA target sites of OsBBM1. Different letters indicate significantly different mean values at p<0.05 (one-way ANOVA with post-hoc Tukey test). FIG. 4C shows a comparison of gR2.0 and gR2.1-mediated activation based on the CRISPR-Act3.0 system in rice protoplasts. The gR2.1 scaffold contains an adenine insertion in the loop of the first aptamer, which was reported in the dCasEV2.1 activation system, which employed a gRNA2.1 scaffold with anchoring sites for VPR (VP64-p65-Rta) transcriptional activator. **p<0.01, two-tailed Student's t-tests. FIG. 4D shows CRISPR-Act3.0-mediated activation of endogenous OsTRR-like and OsCCR1 genes in rice protoplasts. For qRT-PCRassays (FIG. 4B-D), T-DNA vectors without sgRNAs served as the negative control (CTRL). OsTubulin is used as the endogenous control gene. All data are presented as the mean ±s.d. (n=3 independent experiments).

FIG. 5A-E shows multiplexed gene activation by CRISPR-Act3.0 in rice. FIG. 5A is a schematic illustration of assembling sgRNAs for multiplexed gene activation. Multiple sgRNAs are inserted into the Bsal-digested gR2.0 (guide RNA scaffold containing two MS2 RNA aptamers) entry plasmids, respectively, and then assembled using Golden Gate cloning. The final T-DNA expression vector is constructed by Gateway cloning-mediated assembly of dCas9-activator and tRNA-gR2.0 array cassettes into a destination vector of choice. Two to six sgRNAs are easily assembled based on this strategy. Spectinomycin-R, spectinomycin resistance gene. Kanamycin-R, kanamycin resistance gene. RB, right border. LB, left border. Ter, terminator. FIG. 5B shows a comparison of different multiplexed gene activation strategies based on CRISPR-Act3.0 for simultaneous gene activation. I-OsU3, singular gene activation with individual gR2.0 expression cassettes each driven by an OsU3 promoter. M-tRNA, multiple tRNA-mediated gR2.0 expression cassettes driven by a Pol II promoter ZmUbi. M-OsU3, multiple tandem repeats of independent OsU3 based gR2.0 expression cassettes. T-DNA vectors without sgRNAs served as the negative control (CTRL). OsTubulin is used as the endogenous control gene. All data are presented as the mean ±s.d. (n=3 independent experiments). Different letters indicate significantly different mean values at p<0.05 (one-way ANOVA with post-hoc Tukey test). FIG. 5C shows simultaneous activation of multiple enzyme-encoding genes of the proanthocyanidin pathway in rice protoplasts. M-Act3.0, CRISPR-Act3.0-mediated multiplexed gene activation using M-tRNA system. T-DNA vectors without sgRNAs served as the negative control (CTRL). OsTubulin is used as the endogenous control gene. All data are presented as the mean ±s.d. (n=3 independent experiments). FIG. 5D shows expression analysis of proanthocyanidin biosynthetic genes in T0 positive transgenic callus. FIG. 5E shows expression analysis of proanthocyanidin biosynthetic genes in T0 positive transgenic seedlings (leaves). Act3.0-M# represents different transgenic callus (FIG. 5D) and seedlings (FIG. 5E) with CRISPR-Act3.0-mediated multiplexed gene activation using the M-tRNA system. For qRT-PCR assays (FIG. 5E-D), T0 lines containing T-DNA vectors without sgRNAs served as the negative control (CTRL). OsTubulin is used as the endogenous control gene. All data are presented as the mean ±s.d. (n=3 technical replicates).

FIG. 6 is a schematic illustration of assembling sgRNAs for M-U3-based multiplexed gene activation. Multiple sgRNAs are inserted into the BsaI-digested U3 promoter-based gR2.0 (guide RNA scaffold containing two MS2 RNA aptamers) entry plasmids, respectively, and then assembled using Golden Gate cloning. The final T-DNA expression vector is constructed by Gateway cloning-mediated assembly of dCas9-activator and U3-gRNA2.0 cassettes into a destination vector of choice. Two to six sgRNAs are easily assembled based on this strategy. Spectinomycin-R, spectinomycin resistance gene. Kanamycin-R, kanamycin resistance gene. RB, right border. LB, left border. Ter, terminator.

FIG. 7A-B shows a comparison of singular and multiplexed gene activation in rice protoplasts. FIG. 7A-B shows qRT-PCR analysis of singular and multiplexed activation of OsGW7 (FIG. 7A) and OsTPR-like (FIG. 7B). gR1&gR2&gR3, multiplexed assembly of gR1, gR2 and gR3 based on the M-tRNA system. For qRT-PCR assays, T-DNA vectors without sgRNAs served as the negative control (CTRL). OsTubulin is used as the endogenous control gene. All data are presented as the mean ±s.d. (n=3 independent experiments). Different letters indicate significantly different mean values at p<0.05 (one-way ANOVA with post-hoc Tukey test).

FIG. 8A-B shows activation of the β-carotene biosynthesis pathway in rice protoplasts. FIG. 8A shows prescreen individual sgRNAs for gene activation of the β-carotene biosynthesis pathway in rice protoplasts. All data are presented as the mean ±s.d. (n=3 technical replicates). FIG. 8B shows Simultaneous activation of multiple enzyme-encoding genes of the β-carotene pathway in rice protoplasts. All data are presented as the mean ±s.d. (n=3 independent experiments). For qRT-PCR assays, T-DNA vectors without sgRNAs served as the negative control (CTRL). OsTubulin is used as the endogenous control gene.

FIG. 9A-C shows prescreen individual sgRNAs for gene activation of the proanthocyanidin pathway in rice protoplasts. FIG. 9A-B shows an alignment of the promoter sequences of proanthocyanidin biosynthesis pathway genes between Oryza sativa Nipponbare and Kasalath (SEQ ID NOs: 48-54). The promoter regions of proanthocyanidin biosynthesis pathway genes of Kasalath are amplified by PCR and then submitted for Sanger sequencing. The primers are designed based on the genome sequences of Nipponbare. FIG. 9C shows prescreen individual sgRNAs for activation of proanthocyanidin biosynthesis pathway genes in Kasalath. T-DNA vectors without sgRNAs served as the negative control (CTRL). OsTubulin is used as the endogenous control gene. All data are presented as the mean ±s.d. (n=3 technical replicates).

FIG. 10A-C shows activation of regulatory genes in the MBW complex of the proanthocyanidin pathway. FIG. 10A is an alignment of the promoter sequences of the MBW complex genes between Oryza sativa Nipponbare and Kasalath (SEQ ID NOs: 55-58). The promoter regions of the MBW complex genes of Kasalath are amplified by PCR and then submitted for Sanger sequencing. The primers are designed based on the genome sequences of Nipponbare. MBW, MYB-bHLH-WD transcriptional regulatory complex. FIG. 10B shows prescreen individual sgRNAs for activation of the MBW complex genes in Kasalath. All data are presented as the mean ±s.d. (n=3 technical replicates). FIG. 10C shows multiplexed CRISPR-Act3.0 based simultaneous activation of the MBW complex genes in rice protoplasts. All data are presented as the mean ±s.d. (n=3 independent experiments). For qRT-PCR assays (FIG. 10B-C), T-DNA vectors without sgRNAs served as the negative control (CTRL). OsTubulin is used as the endogenous control gene.

FIG. 11A-B shows PCR detection of the CRISPR-Act3.0 components in transgenic callus and seedlings with proanthocyanidin pathway engineering. FIG. 11A-B shows detection of the CRISPR-Act3.0 components in M-Act3.0 transgenic callus (FIG. 11A) and seedlings (FIG. 11B) through PCR. DNA samples were isolated from transgenic callus and seedlings using cetyltrimethylammonium bromide (CTAB) method. Four different pairs of PCR primers were used to detect the related Act3.0 components. M-Act3.0, CRISPR-Act3.0 system-mediated multiple gene activation of the proanthocyanidin pathway. Positive CTRL, A. tumefaciens EHA105 strains containing the M-Act3.0 plasmid used for rice stable transformation. The #1 to #9 indicate individual transgenic lines. T0 lines containing T-DNA vectors without sgRNAs served as the negative control (CTRL). WT, wild type. M, 1 kb DNA ladder (Azura Genomics). One replicate was conducted for the experiments.

FIG. 12A-H shows detection of DNA rearrangements of dpcoCas9- and dzCas9-based CRISPR-Act3.0 systemsin A. tumefaciens EHA105 strains. FIG. 12A-B shows restriction digest analysis of the pLR2858 vector with EcoRI and HindIII using a SnapGene software (FIG. 12A) and in 1% agarose gel (FIG. 12B). The pLR2858 represents the dpcoCas9-based CRISPR-Act3.0 vector targeting the seven enzyme-encoding genes in the β-carotene pathway with M-tRNA expression cassettes. To isolate the pLR2858 plasmid from A. tumefaciens EHA105 strains, plasmid DNAs were isolated from the A. tumefaciens EHA105 strains and introduced into E. coli DH5a, then three to six colonies were picked for each construct. A total of eight tRNAs were expressed in the pLR2858 vector. M, 1 kb DNA ladder (Azura Genomics). Lanes 1 to 6 each represent an individual E. coli DH5a colony. White arrows in FIG. 12B suggests related components were undetectable. FIG. 12C-E shows detection of DNA rearrangements of dpcoCas9-based CRISPR-Act3.0 systems in A. tumefaciens EHA105 strains with different promoters. Different promoters including UBQ10 (ubiquitin-10), ZmUbi and a cauliflower mosaic virus 35S promoter were used to drivethe dpcoCas9 expression, respectively. pLR2633 represents the dpcoCas9-based CRISPR-Act3.0 vector targeting Arabidopsis AtFT and AtTCL1 genes with M-U3 expression cassettes. M, 1 kb DNA ladder (Azura Genomics). White arrows in FIG. 12E suggests related components were undetectable. FIG. 12F-H shows the dzCas9 (a maize codon-optimized dSpCas9) rescues dpcoCas9-Act3.0-mediated DNA rearrangements in A. tumefaciens EHA105 strains. Different promoters including ZmUbi, UBQ10 and a cauliflower mosaic virus 35S promoter were used to drive the dzCas9 expression, respectively. No DNA arrangements were detected. M, 1 kb DNA ladder (Azura Genomics). The pLR2858 and pLR3674 plasmids were digested with EcoRI and HindIII, while other plasmids were digested with only EcoRI. One replicate was conducted for the experiments.

FIG. 13 shows a comparison of the activation efficiency between the dzCas9-Act3.0 and dpcoCas-Act3.0 systems in rice protoplasts. Two individual sgRNAs of OsF3H gR2 and gR3 were used for the comparison assays. T-DNA vectors without sgRNAs served as the negative control (CTRL). OsTubulin is used as the endogenous control gene. All data are presented as the mean ±s.d. (n=3 independent experiments).

FIG. 14A-H shows multiplexed gene activation by CRISPR-Act3.0 in dicot plants. FIG. 14A is a schematic of CRISPR-Act3.0-mediated multiplexed gene activation in Arabidopsis. Both AtFT and AtTCL1 are targeted by two sgRNAs each for activation. gR, single guide RNA. gR2.0, guide RNA scaffold containing two MS2 RNA aptamers. Ter, terminator. FIG. 14B shows the early flowering phenotype in the T1 population of CRISPR-Act3.0 transgenic plants (Act3.0) and no-sgRNA transgenic control plants (CTRL). FIG. 14C shows the number of rosette leaves in the CTRL and the CRISPR-Act3.0 transgenic Arabidopsis plants upon flowering. Boxplot boundaries represent the 25th and 75th percentiles; center lines indicate the medians; whiskers extend 1.5 times the interquartile range from the 25th and 75th percentiles. Individual data points are represented by dots. All data are presented as the mean ±s.d. (n=14 independent plants). FIG. 14D shows that AtFT activation shortens the life cycle of Arabidopsis. C, no-sgRNA transgenic control plants. A, CRISPR-Act3.0 transgenic plants. d, days. Seeds, seeds germinate. Silique, the first silique is produced. Maturing, siliques become maturing. FIG. 14E-F shows analysis of early flowering phenotype and target gene expression (AtFT and AtTCL1) in T2 (FIG. 14E) and T3 (FIG. 14F) generations. Two independent CRISPR-Act3.0 populations along with one CTRL population were shown for each generation. All data are presented as the mean ±s.d. (n=3 technical replicates). EF-1α is used as the endogenous control gene. FIG. 14G shows trichome density on the first two true leaves of Act3.0 transgenic and CTRL plants in both T2 and T3 generations. Two independent CRISPR-Act3.0 populations along with one CTRL population were shown for each generation. All data are presented as the mean ±SE (n=19 and 14 individual plants for T2 and T3 generations, respectively). Different letters indicate significantly different mean values at p<0.05 (one-way ANOVA with post-hoc Tukey test). Bar=0.5 mm. FIG. 14H shows determination of the dzCas9-Act3.0 based activation efficiency in tomato protoplasts. Four individual sgRNAs targeting SFT were designed and tested. T-DNA vectors without sgRNAs served as the negative control (CTRL). SlUbi3 is used as the endogenous control gene. All data are presented as the mean ±s.d. (n=3 independent experiments).

FIG. 15A-B shows the effects of sgRNA position and GC composition for CRISPR-Act3.0-mediated activation in rice. FIG. 15A-B shows gene activation levels in rice cells with different sgRNAs target site positions (FIG. 15A) and GC compositions (FIG. 15B) in rice cells. A total of 56 sgRNAs from 16 different genes are analyzed with ˜3-4 sgRNAs per gene, and each dot indicates one sgRNA. Among these, 26 sgRNAs for 14 different genes target the coding strand, and 30 sgRNAs for 15 different genes target the noncoding strand. A total of 19 sgRNAs from 12 different genes with more than 20-fold activation are included in the dotted box (FIG. 15A). A total of 16 sgRNAs from 11 different genes with more than 20-fold activation are included in the dotted box (FIG. 15B). Black dot, sgRNA targeting coding strand. White dot, sgRNA targeting noncoding strand.

FIG. 16A-G shows expanding the targeting scope of CRISPR-Act3.0. FIG. 16A shows engineering and characterization of four dAaCas12b activation systems based on CRISPR-Act3.0 strategies. Left: schematics of four engineered Cas12b sgRNA scaffolds. Aac.3 and Aa3.8.3 scaffolds each contain one MS2 RNA aptamer. Aac.4 and Aa3.8.5 scaffolds each contain two MS2 RNA aptamers as one MS2 was fused to the 5′ end of the sgRNA scaffold sequence. Right: qRT-PCR data showing targeted activation of OsGW7 and OsBBM1 in rice protoplast cells. A total of five dAaCas12b activation systems are tested, among them, the combination of dAaCas12b-TV-MS2-VPR and Aac.3 sgRNA represents a potent activation system established recently. TV, 6xTAL-VP128. VPR, VP64-p65-Rta. FIG. 16B shows schematic illustrations of the engineered dzCas9-Act3.0, dzCas9-NG-Act3.0 and dSpRY-Act3.0 activation systems. FIG. 16C and FIG. 16E show diagrams of the target site positions of OsGW7 (FIG. 16C) and OsBBM1 (FIG. 16E) for activation. The sgRNAs shown above target the coding strand. The sgRNAs shown below target the noncoding strand. N=A/T/C/G. TSS, transcription start site. FIG. 16D and FIG. 16F show comparisons of dzCas9-Act3.0, dzCas9-NG-Act3.0 and dSpRY-Act3.0-mediated OsGW7 (FIG. 16D) and OsBBM1 (FIG. 16F) activation at NGN PAMs. FIG. 16G shows a comparison of dSpRY-Act3.0-mediated OsBBM1 activation at NAN, NTN, NCN PAMs. For qRT-PCR assays (FIG. 16A, 16D, 16F, 16G), T-DNA vectors without sgRNAs served as the negative control (CTRL). OsTubulin is used as the endogenous control gene. All data are presented as the mean ±s.d. (n=3 independent experiments). Different letters indicate significantly different mean values at p<0.05 (one-way ANOVA with post-hoc Tukey test).

FIG. 17 shows an alignment of Aac.3, Aac.4, Aa3.8.3, and Aa3.8.4 scaffolds (SEQ ID NOs: 59-62). MS2 sequences highlighted in a solid box are copied from the gR2.0 scaffold (guide RNA scaffold containing two MS2 RNA aptamers). MS2 sequences highlighted in a dashed box are copied from the Aa3.8.3 scaffold.

FIG. 18A-C shows the CRISPR-Cas9-Act3.0 system enables flexible switching between genome editing and transcriptional activation by altering single guide RNA (sgRNA) length in rice protoplasts. FIG. 18A shows restriction fragment length polymorphism (RFLP) analysis of Cas9 mediated-genome editing with different length sgRNA. Three biological replicates were performed for each target site. Representative images are shown. The editing efficiencies were quantified using Image LabTM Software (Bio-Rad Laboratories) based on band intensity. CTRL indicates wild type samples. Each dot represents an individual biological replicate. Error bar represents the mean ±s.d. (n=3 independent experiments). FIG. 18B shows a comparison of the editing efficiency between Cas9 and Cas9-Act3.0 with both 20 and 15 nt sgRNAs. Three biological replicates were performed for each target site. Representative images are shown. The editing efficiencies were quantified using Image Lab™ Software (Bio-Rad Laboratories) based on band intensity. Each dot represents an individual biological replicate. Error bar represents the mean ±s.d. (n=3 independent experiments). FIG. 18C shows Cas9-Act3.0-induced gene activation efficiency with different length sgRNAs. T-DNA vectors without sgRNAs served as the negative control (CTRL). OsTubulin was selected as the endogenous control gene. Each dot represents an individual biological replicate. Error bar represents the mean ±s.d. (n=3 independent experiments).

FIG. 19A-F shows characterization of the Cas9- and Cas9n-based CRISPR-Combo systems. FIG. 19A is a schematic illustration of the Cas9-Act3.0 induced-simultaneous gene editing and activation. The Cas9-Act3.0 system consists of a catalytically active Cas9 nuclease and MS2 bacteriophage coat protein (MCP)-SunTag-activators activation complex, and two single guide (sgRNA) scaffolds gR1.0 and gR2.0. Each SunTag peptide recruits 10 copies of activator 2xTAL Activation Domain (TAD) by single-chain variable fragment (scFV) of GCN4 antibody fused to super folder GFP (scFV-sfGFP). gR2.0 contains two MS2 RNA aptamers which bind the MCP-SunTag-2xTAD transcriptional activation complex and activate gene expression with a 15 nt sgRNA and Cas9 nuclease without inducing double-strand breaks (DSB). Simultaneously, gR1.0 induces DSB with a 20 nt sgRNA and Cas9 nuclease. FIG. 19B-C shows Cas9-Act3.0 induces simultaneous gene activation and indel mutation in rice (FIG. 19B) and tomato (FIG. 19C) protoplasts, respectively. One 15 nt sgRNA for both OsBBM1 and SFT was cloned into gR2.0 scaffold for Cas9-Act3.0-mediated gene activation. One 20 nt sgRNA for each OsGW2, OsGN1a and SloyA 7 was cloned into gR1.0 scaffolds for Cas9-Act3.0-mediated genome editing. The dCas9-Act3.0 activation system and Cas9 nuclease are selected as references for comparing Cas9-Act3.0-mediated simultaneous activation and indel mutation efficiency, respectively. FIG. 19D is a schematic representation of the cytidine base editor (CBE)-Cas9n-Act3.0 and adenine base editor (ABE)-Cas9n-Act3.0 induced-simultaneous activation and base editing. CBE-Cas9n-Act3.0 and ABE-Cas9n-Act3.0 system consists of a Cas9 nickase fused with a cytidine or adenine deaminase, a MS2 bacteriophage coat protein (MCP)-SunTag-activators activation complex, and two single guide (sgRNA) scaffolds gR1.0 and gR2.0. Each SunTag peptide recruits 10 copies of activator 2xTAL Activation Domain (TAD) by single-chain variable fragment (scFV) of GCN4 antibody fused to super folder GFP (scFV-sfGFP). gR2.0 contains two MS2 RNA aptamers (in red) which bind the MCP-SunTag-2xTAD transcriptional activation complex and activate gene expression with a 15 nt sgRNA and CBE/ABE-Cas9n without inducing base editing. Simultaneously, gR1.0 induces base editing with a 20 nt sgRNA and CBE/ABE-Cas9n. FIG. 19E-F shows CBE-Cas9n-Act3.0 and ABE-Cas9n-Act3.0 induce simultaneous activation and base editing in rice (FIG. 19E) and tomato (FIG. 19F) protoplasts. One 15 nt sgRNA for both OsBBM1 and SFT was cloned into gR2.0 scaffold for CBE/ABE-Cas9n-Act3.0-mediated gene activation. One 20 nt sgRNA for each OsALS, OsEPSPS and SloyA 7 was cloned into gR1.0 scaffolds for CBE/ABE-Cas9n-Act3.0-mediated base editing. A indicates CBE/ABE-Cas9n-Act3.0 mediates target gene activation with only gR2.0 scaffold. A+BE indicates CBE/ABE-Cas9n-Act3.0 mediates simultaneous activation and base editing with both gR1.0 and gR2.0 scaffolds. To compare base editing efficiency, CBE-Cas9n and ABE-Cas9n are selected as references in CBE-Cas9n-Act3.0 and ABE-Cas9n-Act3.0-mediated A+BE assays, respectively. For the RT-qPCR assays (FIG. 19B-C, FIG. 19E-F), T-DNA vectors without sgRNAs served as the negative control (CTRL). OsTubulin and SlUbi3 are selected as the endogenous control genes for rice and tomato, respectively. Each dot represents an individual biological replicate. Error bar represents the mean ±s.d. (n=3 independent experiments).

FIG. 20A-D shows a comparison of the deletion position and size profiles between Cas9 and Cas9- Act3.0 in rice and tomato protoplasts. FIG. 20A-B shows deletion position analysis of both Cas9 and Cas9-Act3.0 systems in rice (FIG. 20A) and tomato (FIG. 20B) protoplasts based on next-generation sequencing (NGS) data. Deletion frequencies were calculated using the number of reads with deletions at designated nucleotide position divided by the total reads with deletions. Protospacer-adjacent motif (PAM) sequence is highlighted in dashed underline and protospacer sequence is highlighted in solid underline (SEQ ID NOs: 63 and 64). Each dot represents an individual biological replicate. Error bar represents the mean ±s.d. (n=3 independent experiments). FIG. 20C-D shows deletion size analysis of both Cas9 and Cas9-Act3.0 in rice (FIG. 20C) and tomato (FIG. 20D) protoplasts. Deletion frequencies were calculated using the number of reads with designated size deletion divided by the total reads with deletions. Each dot represents an individual biological replicate. Error bar represents the mean ±s.d. (n=3 independent experiments).

FIG. 21A-D shows characterization of the SpRY-based CRISPR-Combo toolkit. FIG. 21A-B shows determination of SpRY-Act3.0 based-simultaneous gene activation (FIG. 21A) and indel mutation (FIG. 21B) in rice protoplasts at both NGG and NGC protospacer-adjacent motifs (PAMs). One 15 nt single guide RNA (sgRNA) of OsBBM1 at both NGG and NGC PAMs was cloned into gR2.0 scaffold for SpRY-Act3.0-mediated gene activation. One 20 nt sgRNA for each OsGW2 and OsGN1a at both NGG and NGC PAMs was cloned into gR1.0 scaffold for SpRY-Act3.0-mediated genome editing. The indel mutation assays were analyzed by next-generation sequencing (NGS). The dSpRY-Act3.0 activation system and SpRY nuclease were selected as references for comparing SpRY-Act3.0-mediated simultaneous gene activation and indel mutation, respectively. T-DNA vectors without sgRNAs served as the negative control (CTRL). OsTubulin was selected as the endogenous control gene. Each dot represents an individual biological replicate. Error bar represents the mean ±s.d. (n=3 independent experiments). FIG. 21C shows deletion position analysis of both Cas9 and Cas9-Act3.0 in rice protoplasts based on NGS data. Deletion frequencies were calculated using the number of reads with deletions at designated nucleotide position divided by the total reads with deletions. PAM sequence is highlighted in dashed underline and protospacer sequence is highlighted in solid underline (SEQ ID NOs: 63 and 65). Each dot represents an individual biological replicate. Error bar represents the mean ±s.d. (n=3 independent experiments). FIG. 21D shows deletion size analysis of both Cas9 and Cas9-Act3.0 systems in rice protoplasts. Deletion frequencies were calculated using the number of reads with designated size deletion divided by the total reads with deletions. Each dot represents an individual biological replicate. Error bar represents the mean ±s.d. (n=3 independent experiments).

FIG. 22A-C shows editing window analysis of CBE- and ABE-Cas9n-Act3.0 base editors in rice and tomato protoplasts. FIG. 22A-B shows editing window analysis of CBE-Cas9n and CBE-Cas9n-Act3.0 base editors in rice (FIG. 22A) and tomato (FIG. 22B) protoplasts based on next-generation sequencing (NGS) data. The C to T conversion efficiencies were analyzed by the CRISPR RGEN tools. Each dot represents an individual biological replicate. Error bar represents the mean ±s.d. (n=3 independent experiments). FIG. 22C shows editing window analysis of ABE-Cas9n and ABE- Cas9n-Act3.0 base editors in rice protoplasts. The A to G conversion efficiencies were analyzed by the CRISPR RGEN tools. Each dot represents an individual biological replicate. Error bar represents the mean ±s.d. (n=3 independent experiments).

FIG. 23A-D shows characterization of the SpRYn-based CRISPR-Combo toolkit. FIG. 23A-B shows determination of CBE-SpRYn-Act3.0 and ABE-SpRYn-Act3.0 based-simultaneous gene activation (FIG. 23A) and base editing (FIG. 23B) efficiency in rice protoplasts. One 15 nt single guide RNA (sgRNA) of OsBBM1 was cloned into gR2.0 scaffold for CBE/ABE- SpRYn-Act3.0-mediated gene activation. One 20 nt sgRNA for both OsALS and OsEPSPS was cloned into gR1.0 scaffold for CBE/ABE-SpRYn-Act3.0-mediated base editing. A indicates CBE/ABE-SpRYn-Act3.0-mediated target gene activation with only gR2.0 scaffold. A+BE indicates CBE/ABE-SpRYn-Act3.0-mediated simultaneous gene activation and base editing with both gR1.0 and gR2.0 scaffolds. To compare base editing efficiency, CBE-SpRYn and ABE-SpRYn were selected as references in CBE-SpRYn-Act3.0 and ABE-SpRYn-Act3.0-mediated A+BE assays, respectively. T-DNA vectors without sgRNAs served as the negative control (CTRL). OsTubulin was selected as the endogenous control gene. Each dot represents an individual biological replicate. Error bar represents the mean ±s.d. (n=3 independent experiments). FIG. 23C-D shows editing window analysis of CBE-SpRYn-Act3.0 (FIG. 23C) and ABE-SpRYn-Act3.0 (FIG. 23D) base editors in rice protoplasts. The C to T and A to G conversion efficiencies were analyzed by the CRISPR RGEN tools. Each dot represents an individual biological replicate. Error bar represents the mean ±s.d. (n=3 independent experiments).

FIG. 24A-H shows CRISPR-Combo systems enable rapid breeding of transgene-free edited plants by promoting flowering. FIG. 24A shows early flowering phenotype is observed in both T1 Cas9-Act3.0-A (activation)+GE (genome editing) and CBE-Cas9n-Act3.0-A (activation)+BE (base editing) transgenic populations. Two AtFT single guide RNAs (sgRNAs) with 15 nt were cloned into gR2.0 scaffolds for Cas9-Act3.0- and CBE-Cas9n-Act3.0-mediated gene activation. One 20 nt sgRNA for both AtPYL1 and AtAP1 was cloned into gR1.0 scaffolds for Cas9-Act3.0-mediated genome editing. One 20 nt sgRNA for both AtALS and AtACC2 was cloned into gR1.0 scaffold for CBE-Cas9n-Act3.0-mediated base editing. Black arrows indicate extra-early flowering plants. FIG. 24B-C shows determination of the efficiency of indel mutation (FIG. 24B) and C to T conversion (FIG. 24C) from T1 extra-early, early, and standard flowering plants using next-generation sequencing (NGS). The flowering phenotype (extra-early, early and standard) was defined as the leaf number when flower buds became visible. Extra-early flowering plants showed four leaves, early flowering plants showed around six to 14 leaves, standard flowering plants showed around 20 to 25 leaves when flower buds became visible. Plants were checked for flower buds every 3 days. Each dot indicates one individual transgenic plant. A total of 53 and 48 independent plants are examined in FIG. 24B and FIG. 24C, respectively. A, activation. GE, genome editing. FIG. 24D shows representative images of extra-early flowering, early flowering, and standard flowering plants in T2 generation. These plants were grown under the same photoperiod and temperature regime. FIG. 24E-F shows segregation analysis of flowering phenotype and transgene-free plants in the T2 Cas9-Act3.0-A+GE (FIG. 24E) and CBE-Cas9n-Act3.0-A+BE (FIG. 24F) populations. A total of six T1 independent extra-early flowering transgenic lines are examined for both Cas9-Act3.0-A+GE- and CBE-Cas9n-Act3.0-A+BE systems. The progenies resulting from self-pollination of the selected extra-early flowering lines are grouped into extra-early flowering, early flowering, and standard flowering plants. The T-DNA region was identified among the T2 standard flowering plants by PCR method. FIG. 24G-H shows mutation analysis of T2 T-DNA free standard flowering populations for Cas9-Act3.0-A+GE (FIG. 24G) and CBE-Cas9n-Act3.0-A+BE (FIG. 2411) systems. The indel frequencies of examined transgene-free plants were determined by NGS. Each dot indicates an individual plant. Error bar represents the mean ±s.d. (n=23 to 41 independent plants).

FIG. 25 shows genotype analysis of Cas9-Act3.0-mediated standard flowering plants (transgene-free) in T2 generation. Representative genotypes of T2 AtPYL1 mutants are displayed (SEQ ID NOs: 66-70). The indel mutations were analyzed by CRISPResso2. Protospacer-adjacent motif (PAM) sequence is highlighted in a solid box. d37, 37 bp deletion. il, i bp insertion. WT, wild type.

FIG. 26A shows genotype analysis of CBE-Cas9n-Act3.0-mediated standard flowering plants (transgene-free) in T2 generation (SEQ ID NOs: 71 and 72). Three kinds of genotypes atalsatacc2, atalsAtACC2 and AtALSatacc2 were detected in CBE-Cas9n-Act3.0-mediated standard flowering (transgene-free) group. Representative genotypes of T2 AtALS and AtACC2 mutants are displayed. Protospacer-adjacent motif (PAM) sequence is in bold. The DNA bases C in the protospacer sequence are underlined. The symbols above the protospacer sequence indicate amino acids. The numbers below the protospacer sequence indicate the percentage of DNA base T or C in total reads. The base editing efficiencies were analyzed by the CRISPR RGEN tools. FIG. 26B shows T3 seedlings of CBE-Cas9n-Act3.0-mediated atalsatacc2 T2 lines exhibited herbicide resistance. Wild-type (WT) and T3 atals atacc2 seeds were cultured on Murashige and Skoog (MS) medium supplemented with a series of concentrations of tribenuron with or without haloxyfop. All seeds were vernalized at 4° C. for two days and then cultured on MS selection medium under a long-day condition (16 h light/8 h dark) at 22° C. for one week. Representative images of WT, 14-#23, 17-#11, and 17-#22 seedlings grown on MS selection medium are shown.

FIG. 27A-B shows determination of Cas9-Act3.0- and CBE-Cas9n-Act3.0-induced potential off-target events at AtFT target sites with 15 nt sgRNA in T2 Arabidopsis plants. FIG. 27A shows identification of Cas9-Act3.0-mediated potential indel mutation with 15 nt sgRNA at two AtFT target sites in both extra-early flowering and T-DNA free standard flowering plants. Three and two independent lines were selected for extra-early flowering and T-DNA free standard flowering groups, respectively. Approximately 15 to 23 individual plants were examined for each line. n/a, no indel mutation was detected. 1d1/1,527, one 1-bp deletion event detected in a total of 1,527 reads. 1d1/838, one 1-bp deletion event detected in a total of 838 reads. The indel mutations were analyzed by CRISPResso2. FIG. 27B shows identification of CBE-Cas9n-Act3.0-mediated potential base editing with 15 nt sgRNA at AtFT-sgRNA1 target site in both extra-early flowering and T-DNA free standard flowering plants. Three and two independent lines were selected for extra-early flowering and T-DNA free standard flowering groups, respectively. CTRL represents wild type plants. Approximately 15 to 24 individual plants were examined for each line. Protospacer-adjacent motif (PAM) sequence of AtFT-sgRNA1 is in bold and DNA bases C in protospacer sequence are underlined (SEQ ID NO: 73). The base editing efficiencies were analyzed by the CRISPR RGEN tools. Each dot represents an individual plant. Error bar represents the mean ±s.d. (n=15 to 24 independent plants). The AtFT-sgRNA2 doesn't contain any DNA base C.

FIG. 28A-J shows the CRISPR-Combo system enables rapid breeding of genome-edited plants by promoting regeneration in poplar. FIG. 28A shows the Cas9-Act3.0 system promotes root initiation and shoot growth by activation of PtWUS in poplar. Two single guide RNAs (sgRNAs) of PtWUS with 15 nt were cloned into gR2.0 scaffolds for Cas9-Act3.0-mediated gene activation. One Pt4CL1 sgRNA with 20 nt was cloned into gR1.0 scaffold for Cas9-Act3.0-mediated genome editing. FIG. 28B shows analysis of the period and rate of root initiation in both Cas9 and Cas9-Act3.0 transgenic populations. Each dot indicates an individual transgenic plant in the upper graph and the mean value of root initiation rate from seven transgenic plants in the lower graph. The root initiation rate was evaluated at the seventh day since these transgenic shoots were transferred to the root induction medium. FIG. 28C shows determination of PtWUS activation level in Cas9-Act3.0-mediated transgenic plants using quantitative real-time RT-PCR (qRT-PCR). Leaf tissue was sampled for total RNA extraction for each examined plant. CTRL represents the randomly selected Cas9-mediated transgenic plant. Error bar represents the mean ±s.d. (n=3 technical replicates). Both PtCDC2 and PtPT1 were used as the endogenous control gene. FIG. 28D shows zygosity analysis of Cas9- and Cas9-Act3.0-mediated T0 Pt4CL1 mutants. The frequencies of each zygotic type are shown as numbers among the overall T0 mutant populations. A total of 20 individual transgenic plants were examined for both Cas9 and Cas9-Act3.0 systems using next-generation sequencing (NGS). FIG. 28E shows representative genotypes of Pt4CL1 mutation in Cas9-Act3.0-mediated PtWUS-overexpressing and Cas9-mediated plants (SEQ ID NOs: 74-78). sgRNA, single guide RNA. PAM, protospacer adjacent motif. FIG. 28F shows the Cas9-Act3.0 system promotes de novo callus formation from stem cuttings by activation of PtWUS. Stem cuttings from CTRL and PtWUS-overexpressing poplar plants were cultured on callus induction medium (CIM) for 20 days. FIG. 28G shows determination of PtWOX11 activation level in Cas9-Act3.0-mediated transgenic plants using qRT-PCR. Two 15 nt sgRNAs of PtWOX11 were cloned into gR2.0 scaffolds for Cas9-Act3.0-mediated gene activation. One Pt4CL1 sgRNA with 20 nt was cloned into gR1.0 scaffold for Cas9-Act3.0-mediated genome editing. Leaf tissue was sampled for total RNA extraction for each examined plant. CTRL represents the randomly selected Cas9-mediated transgenic plant. Error bar represents the mean ±s.d. (n=3 technical replicates). Both PtCDC2 and PtPT1 are used as the endogenous control gene. FIG. 2811 shows zygosity analysis of Pt4CL1 mutation in Cas9-Act3.0-mediated T0 PtWOX11-overexpressing plants. The frequencies of each zygotic type are shown as numbers among the overall T0 mutant population. A total of 10 individual transgenic plants were examined for Cas9-Act3.0 system using NGS. FIG. 28I shows representative genotypes of Pt4CL1 mutation in Cas9-Act3.0-mediated PtWOX11-overexpressing plants (SEQ ID NOs: 74, 75, and 77). FIG. 28J shows the Cas9-Act3.0 system promotes de novo callus formation from stem cuttings by activation of PtWOX11. Stem cuttings from CTRL and PtWOX11-overexpressing poplar plants were cultured on callus induction medium (CIM) for 20 days.

FIG. 29A-B shows the Cas9-Act3.0 system promotes de-novo callus and root organogenesis from leaf and stem cutting explants by activation of PtWUS in poplar. FIG. 29A shows the Cas9-Act3.0 system promotes de-novo callus regeneration of leaf-disc by activation of PtWUS in poplar. leaf-discs from CTRL and PtWUS-overexpressing (#2, #4, #17) poplar plants were cultured on callus induction medium (CIM) with and without hormones for 14 days. FIG. 29B shows the Cas9-Act3.0 system promotes de-novo root initiation and shoot growth of stem cuttings by activation of PtWUS in poplar. Four stem cuttings from both CTRL and PtWUS-overexpressing plants were cultured in one magenta box with root induction medium (RIM). Representative images of root and shoot regeneration are displayed. Cycles indicate the root locations of stem cuttings.

FIG. 30A-C shows the Cas9-Act3.0 system-mediated, simultaneous PtARK1 activation and Pt4CL1 editing in poplar. FIG. 30A shows determination of the PtARK1 activation level in Cas9-Act3.0-mediated transgenic plants using quantitative real-time RT-PCR (qRT-PCR). Two single guide RNAs (sgRNAs) of PtARK1 with 15 nt were cloned into gR2.0 scaffolds for Cas9-Act3.0-mediated gene activation. One Pt4CL1 sgRNA with 20 nt was cloned into gR1.0 scaffold for Cas9-Act3.0-mediated genome editing. Leaf tissue was sampled for total RNA extraction for each examined plant. CTRL represents the randomly selected Cas9-mediated transgenic plants. Error bar represents the mean ±s.d. (n=3 technical replicates). Both PtCDC2 and PtPT1 were used as the endogenous control gene. FIG. 30B shows zygosity analysis of Pt4CL1 mutation in Cas9-Act3.0-mediated PtARK1-overexpressing plants. The frequencies of each zygotic type are shown as numbers among the overall T0 mutant population. A total of 10 individual transgenic plants were examined for Cas9-Act3.0 system using next-generation sequencing (NGS). FIG. 30C shows representative genotypes of Pt4CL1 mutation in Cas9-Act3.0-mediated PtARK1-overexpressing plants (SEQ ID NOs: 74, 75, 77, and 79). sgRNA, single guide RNA. PAM, protospacer-adjacent motif.

FIG. 31A-D shows Cas9-Act3.0-mediated enhancement of tissue culture in dicots and monocots. FIG. 31A shows prescreen individual sgRNAs for gene activation of callus and shoot meristem formation pathways using quantitative real-time RT-PCR (qRT-PCR) in tomato protoplasts. T-DNA vectors without sgRNAs served as the negative control (CTRL). SlUbi3 was selected as the endogenous control gene. Error bar represents the mean ±s.d. (n=3 technical replicates). FIG. 31B shows Cas9-Act3.0 based T-DNA constructs for tomato stable transformation. One 15 nt sgRNA for each SlBBM, SlARF7, SlARF19, SlFAD-BD, SlSTM, SlWUS was cloned into gR2.0 scaffold for Cas9-Act3.0-mediated gene activation. One 20 nt sgRNA of SlPSY was cloned into a gR1.0 scaffold for Cas9-Act3.0-mediated genome editing. The vector 4335 only targeting SlPSY was generated as a control. FIG. 31C shows determination of T-DNA constructs-induced multiplexed gene activation in tomato protoplasts using qRT-PCR. T-DNA vectors without sgRNAs served as the negative control (CTRL). SlUbi3 was selected as the endogenous control gene. Error bar represents the mean ±s.d. (n=3 technical replicates). FIG. 31D shows hormone-free plant regeneration by CRISPR-Combo in rice. Embryogenic callus explants of rice variety Kitaake were inoculated with EHA105:Cas9-Act3.0-GE and two kinds of EHA105:Cas9-Act3.0-A-GE strains, respectively. After cocultivation, inoculated explants were transferred onto regeneration and selection medium (RSM) supplemented with hygromycin and timentin. All media were hormone-free. Representative images of Cas9-Act3.0-GE and Cas9-Act3.0-A-GE calluses grown on RSM are shown. A, activation. GE, genome editing.

FIG. 32A-B shows the CRISPR-Combo systems for simultaneous genome editing, gene activation, and gene repression. FIG. 32A is a schematic illustration of the Cas9-Act3.0 induced-simultaneous genome editing, activation and gene repression. The Cas9-Act3.0 system consists of a catalytically active Cas9 nuclease and MS2 bacteriophage coat protein (MCP)-SunTag-activators activation complex, and two kinds of single guide (sgRNA) scaffolds gR1.0 and gR2.0. Each SunTag peptide recruits 10 copies of activator 2xTAL Activation Domain (TAD) by single-chain variable fragment (scFV) of GCN4 antibody fused to super folder GFP (scFV-sfGFP). gR2.0 contains two MS2 RNA aptamers which bind the MCP-SunTag-2xTAD transcriptional activation complex and activate gene expression with a 15 nt sgRNA and Cas9 nuclease without inducing double-strand break (DSB). gR1.0 induces DSB with a 20 nt sgRNA and Cas9 nuclease. gR1.0 induces gene repression by targeting the transcriptional site (TSS) and 5′ untranslated region (UTR) with a 15 nt sgRNA and Cas9 nuclease without inducing double-strand breaks (DSB). FIG. 32B is a schematic representation of the cytidine base editor (CBE)-Cas9n-Act3.0 and adenine base editor (ABE)-Cas9n-Act3.0 induced-simultaneous base editing, activation and gene repression. The CBE-Cas9n-Act3.0 and ABE-Cas9n-Act3.0 system consists of a Cas9 nickase fused with a cytidine or adenine deaminase, a MS2 bacteriophage coat protein (MCP)-SunTag-activators activation complex, and two kinds of single guide (sgRNA) scaffolds gR1.0 and gR2.0. Each SunTag peptide recruits 10 copies of activator 2xTAL Activation Domain (TAD) by single-chain variable fragment (scFV) of GCN4 antibody fused to super folder GFP (scFV-sfGFP). gR2.0 contains two MS2 RNA aptamers which bind the MCP-SunTag-2xTAD transcriptional activation complex and activate gene expression with a 15 nt sgRNA and CBE/ABE-Cas9n without inducing base editing. Simultaneously, gR1.0 induces base editing with a 20 nt sgRNA and CBE/ABE-Cas9n. gR1.0 induces gene repression by targeting the transcriptional site (TSS) and 5′ untranslated region (UTR) with a 15 nt sgRNA and CBE/ABE-Cas9n without inducing base editing.

FIG. 33A-B shows that dCas9 enables gene repression by binding near the transcription start site (TSS). Prescreen individual sgRNAs for OsKu70 (FIG. 33A) and OsKu80 (FIG. 33B) repression by binding near TSS in rice protoplasts. T-DNA vectors without sgRNAs served as the negative control (CTRL). OsTubulin was selected as the endogenous control gene. Each dot represents an individual biological replicate. Error bar represents the mean ±s.d. (n =3 independent experiments).

### DETAILED DESCRIPTION

A highly robust CRISPRa system, CRISPR-Act3.0, developed through systematically exploring different effector recruitment strategies and various transcription activators is provided. The CRISPR-Act3.0 system results in four- to six-fold higher activation than the state-of-the-art CRISPRa systems. In addition, the CRISPR-Act3.0 allows simultaneous modification of multiple traits, which are stably transmitted to the T3 generations. RNA-guided CRISPR-Cas9 nuclease, its derived base editors, CRISPRa systems, and CRISPRi systems are nearly always used in isolation, leaving their potential combinational power untapped. The present disclosure also provides a versatile CRISPR-Combo platform for simultaneous genome editing, gene activation, and gene repression in plants. Based on a single Cas polypeptide, the multifunctionality of CRISPR-Combo is programmed through sgRNA engineering. Hence, implementation of CRISPR-Combo is as simple as any multiplexed CRISPR systems.

So that the present invention may be more readily understood, certain terms are first defined. Unless defined otherwise, all technical and scientific terms used herein have the same meaning as commonly understood by one skilled in the art to which example embodiments of the invention pertain. Many methods and materials similar, modified, or equivalent to those described herein can be used in the practice of the example embodiments without undue experimentation, the preferred materials and methods are described herein. In describing the example embodiments and claiming the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

The methods, systems, and compositions of the present disclosure may comprise, consist essentially of, or consist of the components described herein. As used herein, “consisting essentially of” means that the methods, systems, and compositions may include additional steps or components, but only if the additional steps or components do not materially alter the basic and novel characteristics of the claimed methods, systems, and compositions.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined approximately 20 nucleotide “spacer” or “targeting” sequence which defines the genomic target to be modified. The genomic target of Cas9 can be changed by changing the targeting sequence present in the sgRNA.

“CRISPRa” system refers to a modification of the CRISPR/Cas system that functions to activate or increase gene expression.

“dCas9” as used herein refers to a catalytically dead Cas9 protein that lacks endonuclease activity.

The term “dead guide RNA” refers to a guide RNA, which is catalytically inactive yet maintains target-site binding capacity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “exogenous” refers to any material introduced from or produced outside an organism, cell, tissue or system.

The term “expression” as used herein is defined as the transcription and/or translation of a particular nucleotide sequence driven by its promoter.

“Isolated” means altered or removed from the natural state. For example, a nucleic acid or a peptide naturally present in a living plant is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

A “vector” is a composition of matter which comprises an isolated nucleic acid and which can be used to deliver the isolated nucleic acid to the interior of a cell.

### Recombinant Polypeptides

The present disclosure relates to the use of recombinant polypeptides to modulate (e.g., activate, repress) expression of a target nucleic acid.

As used herein, a “polypeptide” is an amino acid sequence including a plurality of consecutive polymerized amino acid residues (e.g. at least about 15 consecutive polymerized amino acid residues). “Polypeptide” refers to an amino acid sequence, oligopeptide, peptide, protein, or portions thereof, and the terms “polypeptide” and “protein” are used interchangeably.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

Recombinant polypeptides of the present disclosure that are composed of individual polypeptide domains may be described based on the individual polypeptide domains of the overall recombinant polypeptide. A domain in such a recombinant polypeptide refers to the particular stretches of contiguous amino acid sequences with a particular function or activity. For example, a recombinant polypeptide that is a fusion of a transcriptional activator polypeptide and an affinity polypeptide, the contiguous amino acids that encode the transcriptional activator polypeptide may be described as the transcriptional activator domain in the overall recombinant polypeptide, and the contiguous amino acids that encode the affinity polypeptide may be described as the affinity domain in the overall recombinant polypeptide. Individual domains in an overall recombinant protein may also be referred to as units of the recombinant protein. Recombinant polypeptides that are composed of individual polypeptide domains may also be referred to as fusion polypeptides.

Certain embodiments of the present disclosure relate to a polypeptide comprising an adapter domain and a multimerized epitope domain. In certain embodiments, the adapter domain is recombinantly fused to a multimerized epitope domain (e.g., an adapter-multimerized epitope fusion protein). The adapter domain may be in an N-terminal orientation or a C-terminal orientation relative to the multimerized epitope domain. The multimerized epitope domain may be in an N-terminal orientation or a C-terminal orientation relative to the adapter domain. In some embodiments, an adapter-multimerized epitope fusion protein may be a direct fusion of an adapter domain and a multimerized epitope domain. In some embodiments, an adapter-multimerized epitope fusion protein may be an indirect fusion of an adapter domain and a multimerized epitope domain. In embodiments where the fusion is indirect, a linker domain or other contiguous amino acid sequence may separate the adapter domain and the multimerized epitope domain.

Certain embodiments of the present disclosure relate to a polypeptide comprising an affinity domain and a transcriptional activation domain. In certain embodiments, an affinity domain is recombinantly fused to the transcriptional activator domain (e.g., an affinity-transcriptional activator fusion protein). The transcriptional activator domain of an affinity-transcriptional activator fusion protein may be in an N-terminal orientation or a C-terminal orientation relative to the affinity polypeptide. The affinity polypeptide domain of an affinity-transcriptional activator fusion protein may be in an N-terminal orientation or a C-terminal orientation relative to the transcriptional activator polypeptide domain. In some embodiments, an affinity-transcriptional activator fusion protein may be a direct fusion of an affinity domain and transcriptional activator domain. In some embodiments, an affinity-transcriptional activator fusion protein may be an indirect fusion of an affinity polypeptide domain and a transcriptional activator domain. In embodiments where the fusion is indirect, a linker domain or other contiguous amino acid sequence may separate the affinity domain and the transcriptional activator domain.

### Transcriptional Activators

Certain aspects of the present disclosure involve targeting a transcriptional activator to a target nucleic acid such that the transcriptional activator activates the expression/transcription of the target nucleic acid. In some embodiments, a transcriptional activator is present in a recombinant polypeptide that contains a transcriptional activator polypeptide and an affinity polypeptide.

Transcriptional activators are polypeptides that facilitate the activation of transcription/expression of a nucleic acid (e.g., a gene). Transcriptional activators may be DNA-binding proteins that bind to enhancers, promoters, or other regulatory elements of a nucleic acid, which then promotes expression of the nucleic acid. Transcriptional activators may interact with proteins that are components of transcriptional machinery or other proteins that are involved in regulation of transcription in a manner that promotes expression of the nucleic acid.

Transcriptional activators of the present disclosure may be endogenous to the host plant, or they may be exogenous/heterologous to the host plant. In some embodiments, the transcriptional activator is a viral transcriptional activator. In some embodiments, the transcriptional activator is derived from Herpes Simplex Virus. For example, one or more copies of a Herpes Simplex Virus Viral Protein 16 (VP16) domain may be used herein. In some embodiments, at least two, at least three, or at least four or more copies of a VP16 domain may be used as a transcriptional activator. A polypeptide containing 4 copies of the Herpes Simplex Virus Viral Protein 16 (VP16) domain is known as a VP64 domain.

In some embodiments, the transcriptional activator is a VP64 polypeptide. A VP64 polypeptide of the present disclosure may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 2.

In certain embodiments, the transcriptional activator is a TAL activation domain (TAD) derived from the transcription activator-like effector (TALE) proteins from the plant pathogen Xanthomonas. In some embodiments, the transcriptional activator comprises two repeats of TAD (2xTAD). A TAD polypeptide of the present disclosure may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 4.

Other exemplary transcriptional activators include, for example, the EDLL motif present in the ERF/EREBP family of transcriptional regulators in plants, activation domains of or full-length transcription factors, plant endogenous and exogenous histone acetylases (e.g. p300 from mammals), histone methylases (e.g. H3K4 methylation depositors (SDG2)), histone demethylases (e.g. H3K9 demethylases (IBM1)), Polymerase II subunits, and various combinations of the above mentioned transcriptional activators. For example, 2xTAD and VP64 may each be fused to an affinity polypeptide.

Additional transcriptional activators that may be used in the methods and compositions described herein will be readily apparent to those of skill in the art.

### Affinity Polypeptides

Certain embodiments of the present disclosure relate to recombinant polypeptides that contain an affinity polypeptide. Affinity polypeptides of the present disclosure may bind to one or more epitopes (e.g. a multimerized epitope). In some embodiments, an affinity polypeptide is present in a recombinant polypeptide that contains a transcriptional activator polypeptide and an affinity polypeptide.

A variety of affinity polypeptides are known in the art and may be used herein. Generally, the affinity polypeptide should be stable in the conditions present in the intracellular environment of a plant cell. Additionally, the affinity polypeptide should specifically bind to its corresponding epitope with minimal cross-reactivity.

The affinity polypeptide may be an antibody such as, for example, an scFv. The antibody may be optimized for stability in the plant intracellular environment. When a GCN4 epitope is used in the methods described herein, a suitable affinity polypeptide that is an antibody may contain an anti-GCN4 scFv domain.

In embodiments where the affinity polypeptide is an scFv antibody, the polypeptide may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 14.

Other exemplary affinity polypeptides include, for example, proteins with SH2 domains or the domain itself, 14-3-3 proteins, proteins with SH3 domains or the domain itself, the Alpha-Syntrophin PDZ protein interaction domain, the PDZ signal sequence, or proteins from plants, which can recognize AGO hook motifs (e.g., AGO4 from Arabidopsis thaliana).

### Epitopes and Multimerized Epitopes

Certain embodiments of the present disclosure relate to recombinant polypeptides that contain an epitope or a multimerized epitope. Epitopes of the present disclosure may bind to an affinity polypeptide. In some embodiments, an epitope or multimerized epitope is present in a recombinant polypeptide that contains an adapter polypeptide and an epitope or multimerized epitope.

Epitopes of the present disclosure may be used for recruiting affinity polypeptides (and any polypeptides they may be recombinantly fused to) to an adapter polypeptide. In embodiments where an adapter polypeptide is fused to an epitope or a multimerized epitope, the adapter polypeptide may be fused to one copy of an epitope, multiple copies of an epitope, more than one different epitope, or multiple copies of more than one different epitope as further described herein.

A variety of epitopes and multimerized epitopes are known in the art and may be used herein. In general, the epitope or multimerized epitope may be any polypeptide sequence that is specifically recognized by an affinity polypeptide of the present disclosure. Exemplary epitopes may include a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, a VSV-G epitope, and a GCN4 epitope.

Other exemplary amino acid sequences that may serve as epitopes and multimerized epitopes include, for example, phosphorylated tyrosines in specific sequence contexts recognized by SH2 domains, characteristic consensus sequences containing phosphoserines recognized by 14-3-3 proteins, proline-rich peptide motifs recognized by SH3 domains, the PDZ protein interaction domain or the PDZ signal sequence, and the AGO hook motif from plants.

Epitopes described herein may also be multimerized. Multimerized epitopes may include at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 or more copies of an epitope.

Multimerized epitopes may be present as tandem copies of an epitope, or each individual epitope may be separated from another epitope in the multimerized epitope by a linker or other amino acid sequence. Suitable linker regions are known in the art and are described herein. The linker may be configured to allow the binding of affinity polypeptides to adjacent epitopes without, or without substantial, steric hindrance. Linker sequences may also be configured to provide an unstructured or linear region of the polypeptide to which they are recombinantly fused. The linker sequence may comprise e.g. one or more glycines and/or serines. The linker sequences may be e.g. at least 2, at least 3, at least 4, at least 5, at least 6, at least 7, at least 8, at least 9, or at least 10 or more amino acids in length.

In some embodiments, the epitope is a GCN4 epitope (SEQ ID NO: 16). In some embodiments, the multimerized epitope contains at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 copies of a GCN4 epitope. In some embodiments, the multimerized epitope contains 10 copies of a GCN4 epitope (SEQ ID NO: 18).

### Linkers

Various linkers may be used in the construction of recombinant proteins as described herein. In general, linkers are short peptides that separate the different domains in a multi-domain protein. They may play an important role in fusion proteins, affecting the crosstalk between the different domains, the yield of protein production, and the stability and/or the activity of the fusion proteins. Linkers are generally classified into 2 major categories: flexible or rigid. Flexible linkers are typically used when the fused domains require a certain degree of movement or interaction, and these linkers are usually composed of small amino acids such as, for example, glycine (G), serine (S) or proline (P).

The certain degree of movement between domains allowed by flexible linkers is an advantage in some fusion proteins. However, it has been reported that flexible linkers can sometimes reduce protein activity due to an inefficient separation of the two domains. In this case, rigid linkers may be used since they enforce a fixed distance between domains and promote their independent functions. A thorough description of several linkers has been provided in Chen X et al., 2013, Advanced Drug Delivery Reviews 65 (2013) 1357-1369).

Various linkers may be used in, for example, the construction of recombinant polypeptides as described herein. Linkers may be used in e.g., adapter-multimerized epitope fusion proteins as described herein to separate the coding sequences of the adapter domain and the multimerized epitope domain. Linkers may be used in e.g., affinity-transcriptional activator fusion proteins as described herein to separate the coding sequences of the affinity domain and the transcriptional activator domain. For example, a variety of wiggly/flexible linkers, stiff/rigid linkers, short linkers, and long linkers may be used as described herein. Various linkers as described herein may be used in the construction of recombinant proteins as described herein.

A variety of shorter or longer linker regions are known in the art, for example corresponding to a series of glycine residues, a series of adjacent glycine-serine dipeptides, a series of adjacent glycine-glycine-serine tripeptides, or known linkers from other proteins.

### Nuclear Localization Signals (NLS)

Recombinant polypeptides of the present disclosure may contain one or more nuclear localization signals (NLS). Nuclear localization signals may also be referred to as nuclear localization sequences, domains, peptides, or other terms readily apparent to those of skill in the art. Nuclear localization signals are a translocation sequence that, when present in a polypeptide, direct that polypeptide to localize to the nucleus of a eukaryotic cell.

Various nuclear localization signals may be used in recombinant polypeptides of the present disclosure. For example, one or more SV40-type NLS or one or more REX NLS may be used in recombinant polypeptides. Recombinant polypeptides may also contain two or more tandem copies of a nuclear localization signal. For example, recombinant polypeptides may contain at least two, at least three, at least four, at least five, at least six, at least seven, at least eight, at least nine, or at least ten copies, either tandem or not, of a nuclear localization signal.

Recombinant polypeptides of the present disclosure may contain one or more nuclear localization signals that contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 20.

### Tags, Reporters, and Other Features

Recombinant polypeptides of the present disclosure may contain one or more tags that allow for e.g., purification and/or detection of the recombinant polypeptide. Various tags may be used herein and are well-known to those of skill in the art. Exemplary tags may include HA, GST, FLAG, MBP, etc., and multiple copies of one or more tags may be present in a recombinant polypeptide.

Recombinant polypeptides of the present disclosure may contain one or more reporters that allow for e.g., visualization and/or detection of the recombinant polypeptide. A reporter polypeptide encodes a protein that may be readily detectable due to its biochemical characteristics such as, for example, enzymatic activity or chemifluorescent features. Reporter polypeptides may be detected in a number of ways depending on the characteristics of the particular reporter. For example, a reporter polypeptide may be detected by its ability to generate a detectable signal (e.g., fluorescence), by its ability to form a detectable product, etc. Various reporters may be used herein and are well-known to those of skill in the art. Exemplary reporters may include GFP, GUS, mCherry, luciferase, etc., and multiple copies of one or more tags may be present in a recombinant polypeptide.

Recombinant polypeptides of the present disclosure may contain one or more polypeptide domains that serve a particular purpose depending on the particular goal/need. For example, recombinant polypeptides may contain translocation sequences that target the polypeptide to a particular cellular compartment or area. Suitable features will be readily apparent to those of skill in the art.

### CRISPR/Cas

CRISPR systems naturally use small base-pairing guide RNAs to target and cleave foreign DNA elements in a sequence-specific manner (Wiedenheft et al., 2012). There are diverse CRISPR systems in different organisms that may be used to target proteins of the present disclosure to a target nucleic acid. One of the simplest systems is the type II CRISPR system from Streptococcus pyogenes. Only a single gene encoding the Cas9 protein and two RNAs, a mature CRISPR RNA (crRNA) and a partially complementary trans-acting RNA (tracrRNA), are necessary and sufficient for RNA-guided silencing of foreign DNAs (Jinek et al, 2012). Maturation of crRNA requires tracrRNA and RNase III (Deltcheva et al., 2011). However, this requirement can be bypassed by using an engineered small guide RNA (gRNA) containing a designed hairpin that mimics the tracrRNA-crRNA complex (Jinek et al., 2012). Base pairing between the gRNA and target DNA normally causes double-strand breaks (DSBs) due to the endonuclease activity of Cas9.

It is known that the endonuclease domains of the Cas9 protein can be mutated to create a programmable RNA-dependent DNA-binding protein (dCas9) (Qi et al., 2013). The fact that duplex gRNA-dCas9 binds target sequences without endonuclease activity has been used to tether regulatory proteins, such as transcriptional activators or repressors, to promoter regions in order to modify gene expression (Gilbert et al., 2013), and Cas9 transcriptional activators have been used for target specificity screening and paired nickases for cooperative genome engineering (Mali et al., 2013, Nature Biotechnology 31:833-838). Thus, dCas9 may be used as a modular RNA-guided platform to recruit different proteins to DNA in a highly specific manner.

### Cas Proteins

A variety of Cas proteins may be used in the methods of the present disclosure. There are several Cas9 genes present in different bacteria species (Esvelt, K et al, 2013, Nature Methods). One of the most characterized CAS9 proteins is the CAS9 protein from S. pyogenes that, in order to be active, needs to bind a gRNA with a specific sequence and the presence of a PAM motif (NGG, where N is any nucleotide) at the 3′ end of the target locus. However, other CAS9 proteins from different bacterial species show differences in 1) the sequence of the gRNA they can bind and 2) the sequence of the PAM motif. Therefore, other Cas9 proteins such as, for example, those from Streptococcus thermophilus or N. meningitidis may also be utilized herein. Indeed, these two Cas9 proteins have a smaller size (around 1100 amino acids) as compared to S. pyogenes Cas9 (1400 amino acids), which may confer some advantages during cloning or protein expression.

Cas9 proteins from a variety of bacteria have been used successfully in engineered CRISPR-Cas9 systems. There are also versions of Cas9 proteins available in which the codon usage has been more highly optimized for expression in eukaryotic systems, such as human codon optimized CAS9 (Cell, 152:1173-1183) and plant optimized CAS9 (Nature Biotechnology, 31:688-691).

Cas9 proteins may also be modified for various purposes. For example, Cas9 proteins may be engineered to contain a nuclear-localization sequence (NLS). Cas9 proteins may be engineered to contain an NLS at the N-terminus of the protein, at the C-terminus of the protein, or at both the N- and C-terminus of the protein. Engineering a Cas9 protein to contain an NLS may assist with directing the protein to the nucleus of a host cell. Cas9 proteins may be engineered such that they are unable to cleave nucleic acids (e.g. nuclease-deficient dCas9 polypeptides). One of skill in the art would be able to readily identify a suitable Cas9 protein for use in the methods and compositions of the present disclosure.

Exemplary Cas proteins that may be used in the methods and compositions of the present disclosure may include, for example, a Cas protein having the amino acid sequence of SEQ ID NO: 22, 24, or 26, homologs thereof, and fragments thereof.

In some embodiments, the Cas polypeptide is a SpCas9 polypeptide. SpCas9 polypeptides may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 22.

In some embodiments, the Cas polypeptide is a SpRY polypeptide. SpRY polypeptides may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 24.

In some embodiments, the Cas polypeptide is a AaCas12b polypeptide. AaCas12b polypeptides may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 26.

Fusion proteins comprising a Cas polypeptide and an effector domain are provided. In certain embodiments, the effector domain of the fusion protein can be a nucleotide deaminase or a catalytic domain thereof. The nucleotide deaminase may be an adenosine deaminase or a cytidine deaminase. In general, a Cas polypeptide fused with a deaminase domain can target a sequence in the genome of a plant through the direction of a guide RNA to perform base editing, including the introduction of C to T or A to G substitutions. In some embodiments, the adenosine deaminase can be, without limit, a member of the enzyme family known as adenosine deaminases that act on RNA (ADARs), a member of the enzyme family known as adenosine deaminases that act on tRNA (ADATs), or an adenosine deaminase domain-containing (ADAD) family member. In some embodiments, the cytidine deaminase can be, without limit, a member of the enzyme family known as apolipoprotein B mRNA-editing complex (APOBEC) family deaminase, an activation-induced deaminase (AID), or a cytidine deaminase 1 (CDA1).

An adenosine deaminase domain of the present disclosure may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 33.

A cytidine deaminase domain of the present disclosure may contain an amino acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to the amino acid sequence of SEQ ID NO: 31.

### Guide Polynucleotides

In certain embodiments, the disclosure includes use of “dead guide RNAs”. These 14-nt or 15-nt guide RNAs have been shown to be catalytically inactive yet maintain target-site binding capacity (Kiani et al. (2015) Nat Methods 12, 1051-1054; Dahlman et al. (2015) Nat Biotechnol 33(11): 1159-1161). Thus, these catalytically dead guide RNAs can be utilized to modulate gene expression using a catalytically active Cas nuclease. Therefore, an active Cas nuclease can be repurposed to simultaneously perform genome editing and regulate gene transcription using both types of gRNAs in the same cell using a single active Cas.

In certain embodiments, the guide RNA is provided with one or more distinct RNA loop(s) or distinct sequence(s) (e.g. an aptamer) that can recruit an adapter protein. In particular embodiments, the aptamer is a minimal hairpin aptamer, which selectively binds MS2 bacteriophage coat protein (SEQ ID NO: 36) and is introduced into the guide RNA, such as in the stemloop and/or in a tetraloop.

In some embodiments, the guide RNA comprises an MS2 aptamer having a nucleic acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 34.

A variety of promoters may be used to drive expression of the guide RNA. Guide RNAs may be expressed using a Pol III promoter such as, for example, the U3 promoter, U6 promoter, or the H1 promoter (eLife 2013 2:e00471). For example, an approach in plants has been described using three different Pol III promoters from three different Arabidopsis U6 genes, and their corresponding gene terminators (BMC Plant Biology 2014 14:327). One skilled in the art would readily understand that many additional Pol III promoters could be utilized to simultaneously express many guide RNAs to many different locations in the genome. The use of different Pol III promoters for each gRNA expression cassette may be desirable to reduce the chances of natural gene silencing that can occur when multiple copies of identical sequences are expressed in plants.

In some embodiments, the guide RNA is driven by a U3 promoter. In some embodiments, the guide RNA is driven by a promoter having a nucleic acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 27.

Alternatively, a tRNA-gRNA expression cassette (Xie, X et al, 2015, Proc Natl Acad Sci USA. 2015 Mar. 17; 112(11):3570-5) may be used to deliver multiple gRNAs simultaneously with high expression levels. In such an embodiment, a tRNA in such a cassette may have a nucleic acid sequence with at least about 70%, at least about 75%, at least about 80%, at least about 85%, at least about 90%, at least about 91%, at leak about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 28.

### Recombinant Nucleic Acids Encoding Recombinant Proteins

Certain embodiments of the present disclosure relate to recombinant nucleic acids encoding recombinant proteins of the present disclosure. Certain aspects of the present disclosure relate to recombinant nucleic acids encoding various portions/domains of recombinant proteins of the present disclosure.

As used herein, the terms “polynucleotide,” “nucleic acid,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with analog and inter-nucleotide modifications. As used herein, the symbols for nucleotides and polynucleotides are those recommended by the IUPAC-IUB Commission of Biochemical Nomenclature.

Sequences of the polynucleotides of the present disclosure may be prepared by various suitable methods known in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those skilled in the art and is described in the pertinent texts and literature (e.g., in Matteucci et al., (1980) Tetrahedron Lett 21:719-722; U.S. Pat. Nos. 4,500,707; 5,436,327; and 5,700,637). In addition, the desired sequences may be isolated from natural sources by splitting DNA using appropriate restriction enzymes, separating the fragments using gel electrophoresis, and thereafter, recovering the desired polynucleotide sequence from the gel via techniques known to those skilled in the art, such as utilization of polymerase chain reactions (PCR; e.g., U.S. Pat. No. 4,683,195).

The nucleic acids employed in the methods and compositions described herein may be codon optimized relative to a parental template for expression in a particular host cell. Cells differ in their usage of particular codons, and codon bias corresponds to relative abundance of particular tRNAs in a given cell type. By altering codons in a sequence so that they are tailored to match with the relative abundance of corresponding tRNAs, it is possible to increase expression of a product (e.g., a polypeptide) from a nucleic acid. Similarly, it is possible to decrease expression by deliberately choosing codons corresponding to rare tRNAs. Thus, codon optimization/deoptimization can provide control over nucleic acid expression in a particular cell type (e.g., bacterial cell, plant cell, mammalian cell, etc.). Methods of codon optimizing a nucleic acid for tailored expression in a particular cell type are well-known to those of skill in the art.

Various methods are known to those of skill in the art for identifying similar (e.g. homologs, orthologs, paralogs, etc.) polypeptide and/or polynucleotide sequences, including phylogenetic methods, sequence similarity analysis, and hybridization methods.

Phylogenetic trees may be created for a gene family by using a program such as CLUSTAL (Thompson et al. Nucleic Acids Res. 22: 4673-4680 (1994); Higgins et al. Methods Enzymol 266: 383-402 (1996)) or MEGA (Tamura et al. Mol. Biol. & Evo. 24:1596-1599 (2007)). Once an initial tree for genes from one species is created, potential orthologous sequences can be placed in the phylogenetic tree and their relationships to genes from the species of interest can be determined. Evolutionary relationships may also be inferred using the Neighbor-Joining method (Saitou and Nei, Mol. Biol. & Evo. 4:406-425 (1987)). Homologous sequences may also be identified by a reciprocal BLAST strategy. Evolutionary distances may, for example, be computed using the Poisson correction method (Zuckerkandl and Pauling, pp. 97-166 in Evolving Genes and Proteins, edited by V. Bryson and H. J. Vogel. Academic Press, New York (1965)).

In addition, evolutionary information may be used to predict gene function. Functional predictions of genes can be greatly improved by focusing on how genes became similar in sequence (i.e., by evolutionary processes) rather than on the sequence similarity itself (Eisen, Genome Res. 8: 163-167 (1998)). Many specific examples exist in which gene function has been shown to correlate well with gene phylogeny (Eisen, Genome Res. 8: 163-167 (1998)). By using a phylogenetic analysis, one skilled in the art would recognize that the ability to deduce similar functions conferred by closely-related polypeptides is predictable.

When a group of related sequences are analyzed using a phylogenetic program such as CLUSTAL, closely related sequences typically cluster together or in the same clade (a group of similar genes). Groups of similar genes can also be identified with pair-wise BLAST analysis (Feng and Doolittle, J. Mol. Evol. 25: 351-360 (1987)). Analysis of groups of similar genes with similar functions that fall within one clade can yield sub-sequences that are particular to the clade. These sub-sequences, known as consensus sequences, can not only be used to define the sequences within each chide, but define the functions of these genes; genes within a clade may contain paralogous sequences, or orthologous sequences that share the same function (see also, for example, Mount, Bioinformatics: Sequence and Genome Analysis Cold Spring Harbor Laboratory Press, Cold Spring Harbor, N.Y., page 543 (2001)).

To find sequences that are homologous to a reference sequence, BLAST nucleotide searches can be performed with the BLASTN program, score=100, wordlength=12, to obtain nucleotide sequences homologous to a nucleotide sequence encoding a protein of the disclosure. BLAST protein searches can be performed with the BLASTX program, score=50, wordlength=3, to obtain amino acid sequences homologous to a protein or polypeptide of the disclosure. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, or PSI-BLAST, the default parameters of the respective programs (e.g., BLASTN for nucleotide sequences, BLASTX for proteins) can be used.

Methods for the alignment of sequences and for the analysis of similarity and identity of polypeptide and polynucleotide sequences are well-known in the art.

As used herein “sequence identity” refers to the percentage of residues that are identical in the same positions in the sequences being analyzed. As used herein “sequence similarity” refers to the percentage of residues that have similar biophysical/biochemical characteristics in the same positions (e.g., charge, size, hydrophobicity) in the sequences being analyzed.

Methods of alignment of sequences for comparison are well-known in the art, including manual alignment and computer assisted sequence alignment and analysis. This latter approach is a preferred approach in the present disclosure, due to the increased throughput afforded by computer-assisted methods. As noted below, a variety of computer programs for performing sequence alignment are available or can be produced by one of skill in the art.

The determination of percent sequence identity and/or similarity between any two sequences can be accomplished using a mathematical algorithm. Examples of such mathematical algorithms are the algorithm of Myers and Miller, CABIOS 4:11-17 (1988); the local homology algorithm of Smith et al., Adv. Appl. Math. 2:482 (1981); the homology alignment algorithm of Needleman and Wunsch, J. Mol. Biol. 48:443-453 (1970); the search-for-similarity-method of Pearson and Lipman, Proc. Natl. Acad. Sci. 85:2444-2448 (1988); the algorithm of Karlin and Altschul; Proc. Natl. Acad. Sci. USA 87:2264-2268 (1990), modified as in Karlin and Altschul, Proc. Natl. Acad. Sci. USA 90:5873-5877 (1993).

Computer implementations of these mathematical algorithms can be utilized for comparison of sequences to determine sequence identity and/or similarity. Such implementations include, for example: CLUSTAL in the PC/Gene program (available from Intelligenetics, Mountain View, Calif); the AlignX program, version10.3.0 (Invitrogen, Carlsbad, Calif.) and GAP, BESTFIT, BLAST, FASTA, and TFASTA in the Wisconsin Genetics Software Package, Version 8 (available from Genetics Computer Group (GCG), 575 Science Drive; Madison; Wis., USA). Alignments using these programs can be performed using the default parameters. The CLUSTAL program is well described by Higgins et al. Gene 73:237-244 (1988); Higgins et al. CABIOS 5:151-153 (1989); Corpet et al., Nucleic Acids Res. 16:10881-90 (1988); Huang et al. CABIOS 8:155-65 (1992); and Pearson et al., Meth. Mol. Biol. 24:307-331 (1994). The BLAST programs of Altschul et al. Mol. Biol. 215:403-410 (1990) are based on the algorithm of Karlin and Altschul (1990) supra.

Polynucleotides homologous to a reference sequence can be identified by hybridization to each other under stringent or under highly stringent conditions. Single-stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives; solvents, etc. present in both the hybridization and wash solutions and incubations (and number thereof), as described in more detail in references cited below (e.g., Sambrook et al., Molecular Cloning: A Laboratory Manual, 2nd Ed., Vol. 1-3, Cold Spring Harbor Laboratory, Cold Spring Harbor, N.Y. (“Sambrook”) (1989); Berger and Kimmel, Guide to Molecular Cloning Techniques, Methods in Enzymology, vol. 152 Academic Press, Inc., San Diego, Calif. (“Berger and Kimmel”) (1987); and Anderson and Young, “Quantitative Filter Hybridisation.” In: Flames and Higgins, ed., Nucleic Acid Hybridisation, A Practical Approach. Oxford; TRL Press, 73-111 (1985)).

Encompassed by the disclosure are polynucleotide sequences that are capable of hybridizing to the disclosed polynucleotide sequences and fragments thereof under various conditions of stringency (see, for example, Wahl and Berger, Methods Enzymol. 152: 399-407 (1987); and Kimmel, Methods Enzymo. 152: 507-511, (1987)). Full-length cDNA, homologs, orthologs, and paralogs of polynucleotides of the present disclosure may be identified and isolated using well-known polynucleotide hybridization methods.

With regard to hybridization, conditions that are highly stringent; and means for achieving them, are well known in the art. See, for example, Sambrook et al, (1989) (supra); Berger and Kimmel (1987) pp. 467-469 (supra); and Anderson and Young (1985)(supra).

Hybridization experiments are generally conducted in a buffer of pH between 6.8 to 7.4, although the rate of hybridization is nearly independent of pH at ionic strengths likely to be used in the hybridization buffer (Anderson and Young (1985) (supra)). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments such as genes that duplicate functional enzymes from closely related organisms. The stringency can be adjusted either during the hybridization step or in the post-hybridization washes. Salt concentration, formamide concentration, hybridization temperature and probe lengths are variables that can be used to alter stringency. As a general guideline, high stringency is typically performed at Tₘ—5° C. to Tₘ—20° C., moderate stringency at Tₘ—20° C. to Tₘ—35° C. and low stringency at Tₘ—35° C. to Tₘ—50° C. for duplex >150 base pairs. Hybridization may be performed at low to moderate stringency (25-50° C. below Tₘ), followed by post-hybridization washes at increasing stringencies. Maximum rates of hybridization in solution are determined empirically to occur at Tₘ—25° C. for DNA-DNA duplex and Tₘ—15° C. for RNA-DNA duplex. Optionally, the degree of dissociation may be assessed after each wash step to determine the need for subsequent, higher stringency wash steps.

High stringency conditions may be used to select nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tₘ) for the specific sequence at a defined ionic strength and pH.

Hybridization and wash conditions that may be used to bind and remove polynucleotides with less than the desired homology to the nucleic acid sequences or their complements of the present disclosure include, for example: 6×SSC and 1% SDS at 65° C.;

50% formamide, 4×SSC at 42° C.; 0.5×SSC to 2.0×SSC, 0.1% SDS at 50° C. to 65° C.; or 0.1×SSC to 2×SSC, 0.1% SDS at 50° C.-65° C.; with a first wash step of, for example, 10 minutes at about 42° C. with about 20% (v/v) formamide in 0.1×SSC, and with, for example, a subsequent wash step with 0.2×SSC and 0.1% SUS at 65° C. for 10, 20 or 30 minutes.

For identification of less closely related homologs, wash steps may be performed at a lower temperature, e.g., 50° C. An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min, Wash procedures will generally employ at least two final wash steps. Additional variations on these conditions will be readily apparent to those skilled in the art (see, for example, US Patent Application No. 20010010913).

If desired, one may employ wash steps of even greater stringency, including conditions of 65° C-68° C. in a solution of 15 mM NaCl, 1.5 mM trisodium citrate, and 0.1% SDS, or about 0.2×SSC, 0.1% SDS at 65° C. and washing twice, each wash step of 10, 20 or 30 min in duration, or about 0.1×SSC, 0.1% SDS at 65° C. and washing twice for 10, 20 or 30 min. Hybridization stringency may be increased further by using the same conditions as in the hybridization steps, with the wash temperature raised about 3° C. to about 5° C., and stringency may be increased even further by using the same conditions except the wash temperature is raised about 6° C. to about 9° C.

### Target Nucleic Acids

Various types of nucleic acids may be targeted for gene editing, activation, and repression as will be readily apparent to one of skill in the art. The gene editing may include non-homologous end joining (NHEJ) based mutagenesis (e.g. deletions and insertions; indels), base editing, prime editing, and homology-based repair (HDR). The target nucleic acid may be located within the coding region of a target gene or upstream or downstream thereof. Moreover, the target nucleic acid may reside endogenously in a target gene or may be inserted into the gene, e.g., heterologous, for example, using techniques such as homologous recombination. For example, a target gene of the present disclosure can be operably linked to a control region, such as a promoter, that contains a sequence that can be recognized by e.g., a guide RNA of the present disclosure such that a transcriptional activator of the present disclosure may be targeted to that sequence. In some embodiments, the target nucleic acid is not a target of and/or does not naturally associate with the naturally-occurring transcriptional activator polypeptide.

In some embodiments, the target nucleic acid is endogenous to the plant where the expression of one or more genes is activated according to the methods described herein. In some embodiments, the target nucleic acid is a transgene of interest that has been inserted into a plant. Methods of introducing transgenes into plants are well known in the art. Transgenes may be inserted into plants in order to provide a production system for a desired protein, or may be added to the genetic complement in order to modulate the metabolism of a plant.

Suitable target nucleic acids will be readily apparent to one of skill in the art depending on the particular need or outcome. The target nucleic acid may be in e.g., a region of euchromatin (e.g. highly expressed gene), or the target nucleic acid may be in a region of heterochromatin (e.g. centromere DNA). Use of transcriptional activators according to the methods described herein to induce transcriptional activation in a region of heterochromatin or other highly methylated region of a plant genome may be especially useful in certain research embodiments. For example, activation of a retrotransposon in a plant genome may find use in inducing mutagenesis of other genomic regions in that genome. A target nucleic acid of the present disclosure may be methylated or it may be unmethylated.

The CRISPRa system enables simultaneous activation of many genes in plants and can be used in applications such as: activation of plant endogenous morphogenic genes such as BABY BOOM (BBM) and WUSCHEL (WUS) for promoting plant species or genotype-independent regeneration, a bottleneck to generate transgenic or gene-edited crops; activation of endogenous florigen gene(s) (e.g. FT) for early flowering in plants; activation of morphogenic genes and florigen genes to promote rapid plant regeneration and shorten the plant life cycle for in crop breeding; activation of plant endogenous metabolic pathway genes for improving the production of certain metabolites and creating nutritious foods to improve human health; activation of plant immune responsive genes, especially through a pathogen inducible fashion, to confer designated resistance to plant diseases such as rice blast disease, soybean rust disease and citrus greening or Huanglongbing (HLB) disease; activation of plant enzyme genes for herbicide resistance (examples include ALS activation for resistance to imidazolinone and sulfonylurea, EPSPS activation for resistance glyphosate, ACC activation for resistance to haloxyfop-R-methyl and quizalofop, TubA2 activation for trifluralin, GS2 activation for glufosinate, and CESA3 activation for C17); activation of plant specific development pathways to promote growth, high yield, changed aboveground morphology, altered root structures, climate-resistance, etc; activation of pathways for improved nutrition deposition in plant cell, tissues, and organs including leaves, fruits, roots and seeds in a cell- or tissues-specific manner; activation of C4 photosynthesis pathway genes in C3 plants for improved photosynthesis; activation of plant stresses responsive pathways, especially through a stress inducible fashion, to confer designated resistance to environmental stresses such as heat, cold, drought stresses, etc, to achieve climate resilience.

The CRISPR-Combo system enables simultaneous gene editing, activation, and repression. The technology can be used in many applications such as: simultaneous gene editing and morphogenic genes (e.g. BBM and WUS) activation in crops, which allows for accelerated regeneration of gene-edited crops; simultaneous gene editing and florigen genes (e.g. FT) activation, which allows for fast-track breeding of gene-edited crop products; simultaneous gene editing and activation of morphogenic genes and florigen genes to promote plant regeneration and shorten the juvenile stage to accelerate the gene-editing based crop production pipeline; simultaneous gene editing and activation of an endogenous herbicide resistance gene or an endogenous marker gene to generated genome-edited plants without the use of a conventional selection marker that is provided exogenously (e.g., part of the T-DNA vector) such as nptII, hpt, bar, and gox, that confer resistance to kanamycin, hygromycin, phosphinothricin, and glyphosate, respectively; simultaneous gene editing and activation of one or many metabolic pathways in plant for sophisticated crop engineering; simultaneous gene editing and transcriptional regulation of one or many metabolic pathways in plant in a spatiotemporal manner; simultaneous gene editing and self-activation of the CRISPR-Combo components, through a positive regulation feedback loop, for robust expression in plant cells; simultaneous gene editing and repression of a DNA repair pathway (NHEJ or HDR) to control the gene editing outcomes; simultaneous gene editing and activation of a DNA repair pathway (NHEJ or HDR) to control the gene editing outcomes; simultaneous gene editing, activation of a DNA repair pathway and repression of another DNA repair pathway to control the gene editing outcomes; simultaneous gene editing, activation of morphogenic and/or florigen genes, and repression of a DNA repair pathway in plants; simultaneous gene editing to destroy invading DNA viruses in plants, with concurrent activation of plant defense pathways for engineering synergistic and robust virial defense in plants; simultaneous gene activation and editing of the same enzyme genes to engineer super herbicide resistance in crops.

### Plants of the Present Disclosure

As used herein, a “plant” refers to any of various photosynthetic, eukaryotic multi-cellular organisms of the kingdom Plantae, characteristically producing embryos, containing chloroplasts, having cellulose cell walls and lacking locomotion. As used herein, a “plant” includes any plant or part of a plant at any stage of development, including seeds, suspension cultures, plant cells, embryos, meristematic regions, callus tissue, leaves, roots, shoots, gametophytes, sporophytes, pollen, microspores, and progeny thereof. Also included are cuttings, and cell or tissue cultures. As used in conjunction with the present disclosure, plant tissue includes, for example, whole plants, plant cells, plant organs, e.g., leaves, stems, roots, meristems, plant seeds, protoplasts, callus, cell cultures, and any groups of plant cells organized into structural and/or functional units.

Any plant cell may be used in the present disclosure so long as it remains viable after being transformed with a sequence of nucleic acids. Preferably, the plant cell is not adversely affected by the transduction of the necessary nucleic acid sequences, the subsequent expression of the proteins or the resulting intermediates.

As disclosed herein, a broad range of plant types may be modified to incorporate recombinant polypeptides and/or polynucleotides of the present disclosure. Suitable plants that may be modified include both monocotyledonous (monocot) plants and dicotyledonous (dicot) plants.

Examples of suitable plants may include, for example, species of the Family Gramineae, including Sorghum bicolor and Zea mays; species of the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Cichorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

In some embodiments, plant cells may include, for example, those from corn (Zea mays), canola (Brassica napus, Brassica rapa ssp.), Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), duckweed (Lemna), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatas), cassava (Manihot esculenta), coffee (Coffea spp.), coconut (Cocos nucijra), pineapple (Ananas comosus), citrus trees (Citrus spp.), cocoa (Theobroma cacao), tea (Camellia sinensis), banana (Musa spp.), avocado (Persea americana), fig (Ficus carica), guava (Psidium guajava), mango (Mangifera indica), olive (Olea europaea), Papaya (Carica papaya), cashew (Anacardium occidentale), Macadamia (Macadamia spp.), almond (Prunus amygdalus), sugar beets (Beta vulgaris), sugarcane (Saccharum spp), oats, barley, vegetables, ornamentals, and conifers.

Examples of suitable vegetable plants may include, for example, tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Examples of suitable ornamental plants may include, for example, azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Examples of suitable conifer plants may include, for example, loblolly pine (Pinus taeda), slash pine (Pinus elliottii), Ponderosa pine (Pinus ponderosa), lodgepole pine (Pinus contorta), Monterey pine (Pinus radiata), Douglas-fir (Pseudotsuga menziesii), Western hemlock (Tsuga canadensis), Sitka spruce (Picea glauca), redwood (Sequoia sempervirens), silver fir (Abies amabilis), balsam fir (Abies balsamea), Western red cedar (Thuja plicata), and Alaska yellow-cedar (Chamaecyparis nootkatensis).

Examples of suitable leguminous plants may include, for example, guar, locust bean, fenugreek, soybean, garden beans, cowpea, mungbean, lima bean, fava bean, lentils, chickpea, peanuts (Arachis sp.), crown vetch (Vicia sp.), hairy vetch, adzuki bean, lupine (Lupinus sp.), trifolium, common bean (Phaseolus sp.), field bean (Pisum sp.), clover (Melilotus sp.) Lotus, trefoil, lens, and false indigo.

Examples of suitable forage and turf grass may include, for example, alfalfa (Medicago ssp.), orchard grass, tall fescue, perennial ryegrass, creeping bentgrass, and redtop.

Examples of suitable crop plants and model plants may include, for example, Arabidopsis, corn, rice, alfalfa, sunflower, canola, soybean, cotton, peanut, sorghum, wheat, and tobacco.

The plants of the present disclosure may be genetically modified in that recombinant nucleic acids have been introduced into the plants, and as such the genetically modified plants do not occur in nature. A suitable plant of the present disclosure is one capable of expressing one or more nucleic acid constructs encoding one or more recombinant proteins.

As used herein, the terms “transgenic plant” and “genetically modified plant” are used interchangeably and refer to a plant, which contains within its genome a recombinant nucleic acid. Generally, the recombinant nucleic acid is stably integrated within the genome such that the polynucleotide is passed on to successive generations. However, in certain embodiments, the recombinant nucleic acid is transiently expressed in the plant. The recombinant nucleic acid may be integrated into the genome alone or as part of a recombinant expression cassette. “Transgenic” is used herein to include any cell, cell line, callus, tissue, plant part or plant, the genotype of which has been altered by the presence of exogenous nucleic acid including those transgenics initially so altered as well as those created by sexual crosses or asexual propagation from the initial transgenic.

“Recombinant nucleic acid” or “heterologous nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure describes the introduction of an expression vector into a plant cell, where the expression vector contains a nucleic acid sequence coding for a protein that is not normally found in a plant cell or contains a nucleic acid coding for a protein that is normally found in a plant cell but is under the control of different regulatory sequences. With reference to the plant cell's genome, then, the nucleic acid sequence that codes for the protein is recombinant. A protein that is referred to as recombinant generally implies that it is encoded by a recombinant nucleic acid sequence which may be present in the plant cell. Recombinant proteins of the present disclosure may also be exogenously supplied directly to host cells (e.g. plant cells).

A “recombinant” polypeptide, protein, or enzyme of the present disclosure, is a polypeptide, protein, or enzyme that may be encoded by a “recombinant nucleic acid” or “heterologous nucleic acid” or “recombinant polynucleotide.”

In some embodiments, the genes encoding the recombinant proteins in the plant cell may be heterologous to the plant cell. In certain embodiments, the plant cell does not naturally produce one or more polypeptides of the present disclosure, and contains heterologous nucleic acid constructs capable of expressing one or more genes necessary for producing those molecules. In certain embodiments, the plant cell does not naturally produce one or more polypeptides of the present disclosure, and is provided the one or more polypeptides through exogenous delivery of the polypeptides directly to the plant cell without the need to express a recombinant nucleic acid encoding the recombinant polypeptide in the plant cell.

Recombinant nucleic acids and/or recombinant proteins of the present disclosure may be present in host cells (e.g. plant cells). In some embodiments, recombinant nucleic acids are present in an expression vector, and the expression vector may be present in host cells (e.g. plant cells).

### Expression of Recombinant Proteins in Plants

Recombinant polypeptides of the present disclosure may be introduced into plant cells via any suitable methods known in the art. For example, a recombinant polypeptide can be exogenously added to plant cells and the plant cells are maintained under conditions such that the recombinant polypeptide is involved with targeting one or more target nucleic acids to activate the expression of the target nucleic acids in the plant cells. Alternatively, a recombinant nucleic acid encoding a recombinant polypeptide of the present disclosure can be expressed in plant cells. Additionally, in some embodiments, a recombinant polypeptide of the present disclosure may be transiently expressed in a plant via viral infection of the plant. Methods of introducing recombinant proteins via viral infection or via the introduction of RNAs into plants are well known in the art. For example, Tobacco Rattle Virus (TRV) has been successfully used to introduce zinc finger nucleases in plants to cause genome modification (“Nontransgenic Genome Modification in Plant Cells”, Plant Physiology 154:1079-1087 (2010)).

A recombinant nucleic acid encoding a recombinant polypeptide of the present disclosure can be expressed in a plant with any suitable plant expression vector. Typical vectors useful for expression of recombinant nucleic acids in higher plants are well known in the art and include, for example, vectors derived from the tumor-inducing (Ti) plasmid of Agrobacterium tumefaciens (e.g., see Rogers et al., Meth. in Enzymol. (1987) 153:253-277). These vectors are plant integrating vectors in that on transformation, the vectors integrate a portion of vector DNA into the genome of the host plant. Exemplary A. tumefaciens vectors useful herein are plasmids pKYLX6 and pKYLX7 (e.g., see of Schardl et al., Gene (1987) 61:1-11; and Berger et al., Proc. Natl. Acad. Sci. USA (1989) 86:8402-8406); and plasmid pBI 101.2 that is available from Clontech Laboratories, Inc. (Palo Alto, Calif.).

In addition to regulatory domains, recombinant polypeptides of the present disclosure can be expressed as a fusion protein that is coupled to, for example, a maltose binding protein (“MBP”), glutathione S transferase (GST), hexahistidine, c-myc, or the FLAG epitope for ease of purification, monitoring expression, or monitoring cellular and subcellular localization.

Moreover, a recombinant nucleic acid encoding a recombinant polypeptide of the present disclosure can be modified to improve expression of the recombinant protein in plants by using codon preference. When the recombinant nucleic acid is prepared or altered synthetically, advantage can be taken of known codon preferences of the intended plant host where the nucleic acid is to be expressed. For example, recombinant nucleic acids of the present disclosure can be modified to account for the specific codon preferences and GC content preferences of monocotyledons and dicotyledons, as these preferences have been shown to differ (Murray et al., Nucl. Acids Res. (1989) 17: 477-498).

The present disclosure further provides expression vectors encoding recombinant polypeptides of the present disclosure. A nucleic acid sequence coding for the desired recombinant nucleic acid of the present disclosure can be used to construct a recombinant expression vector, which can be introduced into the desired host cell. A recombinant expression vector will typically contain a nucleic acid encoding a recombinant protein of the present disclosure, operably linked to transcriptional initiation regulatory sequences which will direct the transcription of the nucleic acid in the intended host cell, such as tissues of a transformed plant. Recombinant nucleic acids e.g. encoding recombinant polypeptides of the present disclosure may be expressed on multiple expression vectors or they may be expressed on a single expression vector.

For example, plant expression vectors may include (1) a cloned gene under the transcriptional control of 5 and 3′ regulatory sequences and (2) a dominant selectable marker. Such plant expression vectors may also include, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

In some embodiments, expression of a nucleic acid of the present disclosure may be driven (in operable linkage) with a promoter (e.g. a promoter functional in plants or a plant-specific promoter). A plant promoter, or functional fragment thereof, can be employed to control the expression of a recombinant nucleic acid of the present disclosure in regenerated plants. The selection of the promoter used in expression vectors will determine the spatial and temporal expression pattern of the recombinant nucleic acid in the modified plant, e.g., the nucleic acid encoding the recombinant polypeptide of the present disclosure is only expressed in the desired tissue or at a certain time in plant development or growth. Certain promoters will express recombinant nucleic acids in all plant tissues and are active under most environmental conditions and states of development or cell differentiation (i.e., constitutive promoters). Other promoters will express recombinant nucleic acids in specific cell types (such as leaf epidermal cells, mesophyll cells, root cortex cells) or in specific tissues or organs (roots, leaves or flowers; for example) and the selection will reflect the desired location of accumulation of the gene product. Alternatively, the selected promoter may drive expression of the recombinant nucleic acid under various inducing conditions.

Examples of suitable constitutive promoters may include, for example, the core promoter of the Rsyn7, the core CaMV 355 promoter (Odell et al., Nature (1985) 313:810-812), CaMV 19S (Lawton et al., 1987), rice actin (Wang et al., 1992; U.S. Pat. No. 5,641,876; and McElroy et al., Plant Cell (1985) 2:163-171); ubiquitin (Christensen et al., Plant Mol. Biol. (1989) 12:619-632; and Christensen et al., Plant Mol. Biol. (1992) 18:675-689), pEMU (Last et al., Theor. Appl. Genet. (1991) 81:581-588), MAS (Velton et al., EMBO J. (1984) 3:2723-2730), nos (Ebert et al., 1987), Adh (Walker et al.; 1987), the P- or 2′-promoter derived from T-DNA of Agrobacterium tumefaciens, the Smas promoter, the cinnamyl alcohol dehydrogenase promoter (U.S. Pat. No. 5,683,439), the Nos promoter, the pEmu promoter, the rubisco promoter, the GRP 1-8 promoter, and other transcription initiation regions from various plant genes known to those of skilled artisans, and constitutive promoters described in, for example, U.S. Pat. Nos. 5,608,149; 5,608,144; 5,604,121; 5,569,597; 5,466,785; 5,399,680; 5,268,463; and 5,608,142.

In some embodiments, expression of a nucleic acid of the present disclosure may be driven (in operable linkage) with a UBQ10 promoter. In some embodiments, expression of a nucleic acid of the present disclosure may be driven (in operable linkage) with a promoter having a nucleic acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of SEQ ID NO: 29.

Examples of suitable tissue specific promoters may include, for example, the lectin promoter (Vodkin et al., 1983; Lindstrom et al., 1990), the corn alcohol dehydrogenase 1 promoter (Vogel et al., 1989; Dennis et al., 1984), the corn light harvesting complex promoter (Simpson, 1986; Bansal et al., 1992); the corn heat shock protein promoter (Odell et al., Nature (1985) 313:810-812; Rochester et al., 1986), the pea small subunit RuBP carboxylase promoter (Poulsen et al., 1986; Cashmore et al., 1983), the Ti plasmid mannopine synthase promoter (Langridge et al., 1989), the Ti plasmid nopaline synthase promoter (Langridge et al., 1989), the petunia chalcone isomerase promoter (Van Tunen et al., 1988), the bean glycine rich protein 1 promoter (Keller et al., 1989), the truncated CaMV 35s promoter (Odell et al., Nature (1985) 313:810-812), the potato patatin promoter (Wenzler et al., 1989), the root cell promoter (Conkling et al., 1990); the maize zein promoter (Reina et al., 1990; Kriz et al., 1987; Wandelt and Feix, 1989; Langridge and Feix, 1983; Reina et al., 1990), the globulin-1 promoter (Belanger and Kriz et al., 1991), the a-tubulin promoter, the cab promoter (Sullivan et al., 1989), the PEPCase promoter (Hudspeth & Grula, 1989), the R gene complex-associated promoters (Chandler et al., 1989), and the chalcone synthase promoters (Franken et al., 1991).

Alternatively, the plant promoter can direct expression of a recombinant nucleic acid of the present disclosure in a specific tissue or may be otherwise under more precise environmental or developmental control. Such promoters are referred to here as “inducible” promoters. Environmental conditions that may affect transcription by inducible promoters include, for example, pathogen attack, anaerobic conditions, or the presence of light. Examples of inducible promoters include, for example, the AdhI promoter which is inducible by hypoxia or cold stress; the Hsp70 promoter which is inducible by heat stress, and the PPDK promoter which is inducible by light. Examples of promoters under developmental control include, for example, promoters that initiate transcription only, or preferentially, in certain tissues, such as leaves, roots, fruit, seeds, or flowers. An exemplary promoter is the anther specific promoter 5126 (U.S. Pat. Nos. 5,689,049 and 5,689,051). The operation of a promoter may also vary depending on its location in the genome. Thus, an inducible promoter may become fully or partially constitutive in certain locations.

Moreover, any combination of a constitutive or inducible promoter, and a non-tissue specific or tissue specific promoter may be used to control the expression of various recombinant polypeptides of the present disclosure.

The recombinant nucleic acids of the present disclosure and/or a vector housing a recombinant nucleic acid of the present disclosure, may also contain a regulatory sequence that serves as a 3′ terminator sequence. One of skill in the art would readily recognize a variety of terminators that may be used in the recombinant nucleic acids of the present disclosure. For example, a recombinant nucleic acid of the present disclosure may contain a 3′ NOS terminator.

In some embodiments, recombinant nucleic acids of the present disclosure contain a transcriptional termination site. Transcription termination sites may include, for example, OCS terminators and NOS terminators.

In some embodiments, the vector comprises a nucleic acid sequence with at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% nucleic acid sequence identity to the nucleic acid sequence of any of SEQ ID NOs: 37-47.

Plant transformation protocols as well as protocols for introducing recombinant nucleic acids of the present disclosure into plants may vary depending on the type of plant or plant cell, e.g., monocot or dicot, targeted for transformation. Suitable methods of introducing recombinant nucleic acids of the present disclosure into plant cells and subsequent insertion into the plant genome include, for example, microinjection (Crossway et al, Biotechniques (1986) 4:320-334), electroporation (Riggs et al., Proc. Natl. Acad Sci. USA (1986) 83:5602-5606), Agrobacterium-mediated transformation (U.S. Pat. No. 5,563,055), direct gene transfer (Paszkowski et al., EMBO J. (1984) 3:2717-2722), and ballistic particle acceleration (U.S. Pat. No. 4,945,050; Tomes et al. (1995). “Direct DNA Transfer into Intact Plant Cells via Microprojectile Bombardment,” in Plant Cell, Tissue, and Organ Culture: Fundamental Methods; ed. Gamborg and Phillips (Springer-Verlag, Berlin); and McCabe et al., Biotechnology (1988) 6:923-926).

Additionally, recombinant polypeptides of the present disclosure can be targeted to a specific organelle within a plant cell. Targeting can be achieved by providing the recombinant protein with an appropriate targeting peptide sequence. Examples of such targeting peptides include, for example, secretory signal peptides (for secretion or cell wall or membrane targeting), plastid transit peptides, chloroplast transit peptides, mitochondrial target peptides, vacuole targeting peptides, nuclear targeting peptides, and the like (e.g., see Reiss et al., Mol. Gen. Genet. (1987) 209(1):116-121; Settles and Martienssen, Trends Cell Biol (1998) 12:494-501; Scott et al, J Biol Chem (2000) 10:1074; and Luque and Correas, J Cell Sci (2000) 113:2485-2495).

The modified plant may be grown into plants in accordance with conventional ways (e.g. McCormick et al., Plant Cell. Reports (1986) 81-84). These plants may then be grown, and pollinated with either the same transformed strain or different strains, with the resulting progeny having the desired phenotypic characteristic. Two or more generations may be grown to ensure that the subject phenotypic characteristic is stably maintained and inherited and then seeds harvested to ensure the desired phenotype or other property has been achieved.

The present disclosure also provides plants derived from plants having increased expression, reduced expression, or a genomic edit as a consequence of the methods of the present disclosure. A plant having increased expression, reduced expression, or a genomic edit as a consequence of the methods of the present disclosure may be crossed with itself or with another plant to produce an F1 plant. In some embodiments, one or more of the resulting F1 plants can also have increased expression, reduced expression, or a genomic edit of the target nucleic acid.

Further provided are methods of screening plants derived from plants having increased expression, reduced expression, or a genomic edit as a consequence of the methods of the present disclosure. In some embodiments, the derived plants (e.g. F1 or F2 plants resulting from or derived from crossing the plant having increased expression, reduced expression, or a genomic edit as a consequence of the methods of the present disclosure with another plant) can be selected from a population of derived plants. For example, provided are methods of selecting one or more of the derived plants that (i) lack recombinant nucleic acids, and (ii) have increased expression, reduced expression, or a genomic edit of the target nucleic acid.

### Modulating Expression of a Target Nucleic Acid in Plants

A target nucleic acid of the present disclosure in a plant cell of the present disclosure may have its expression increased/upregulated/activated by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control.

A target nucleic acid of the present disclosure in a plant cell of the present disclosure may have its expression reduced/downregulated/repressed by at least about 5%, at least about 10%, at least about 15%, at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% as compared to a corresponding control.

Various controls will be readily apparent to one of skill in the art. For example, a control may be a corresponding plant or plant cell that does not contain recombinant polypeptides of the present disclosure (e.g. wild-type plant or plant cell).

Methods of probing the expression level of a nucleic acid are well-known to those of skill in the art. For example, qRT-PCR analysis may be used to determine the expression level of a population of nucleic acids isolated from a nucleic acid-containing sample (e.g., plants, plant tissues, or plant cells).

Growing conditions sufficient for the recombinant polypeptides of the present disclosure to be expressed in the plant to be targeted to and modulate the expression of one or more target nucleic acids of the present disclosure are well known in the art and include any suitable growing conditions disclosed herein. Typically, the plant is grown under conditions sufficient to express a recombinant polypeptide of the present disclosure, and for the expressed recombinant polypeptides to be localized to the nucleus of cells of the plant in order to be targeted to and modulate the expression of the target nucleic acids (if those targets are present in the nucleus). Generally, the conditions sufficient for the expression of the recombinant polypeptide will depend on the promoter used to control the expression of the recombinant polypeptide. For example, if an inducible promoter is utilized, expression of the recombinant polypeptide in a plant will require that the plant be grown in the presence of the inducer.

Growing conditions sufficient for the recombinant polypeptides of the present disclosure to be expressed in the plant to be targeted to and modulate the expression of one or more target nucleic acids may vary depending on a number of factors (e.g. species of plant, use of inducible promoter, etc.). Suitable growing conditions may include, for example, ambient environmental conditions, standard greenhouse conditions, growth in long days under standard environmental conditions (e.g. 16 hours of light, 8 hours of dark), growth in 12 hour light: 12 hour dark day/night cycles, etc.

Various time frames may be used to observe changes in expression of a target nucleic acid according to the methods of the present disclosure. Plants may be observed/assayed for changes in expression of a target nucleic acid after, for example, about 5 days of growth, about 10 days of growth, about 15 days after growth, about 20 days after growth, about 25 days after growth, about 30 days after growth, about 35 days after growth, about 40 days after growth, about 50 days after growth, or 55 days or more of growth.

### Embodiments

The following numbered embodiments also form part of the present disclosure:
1. A system for activating expression of a target nucleic acid, the system comprising: (i) a Cas polypeptide, or a polynucleotide encoding the Cas polypeptide; (ii) a guide polynucleotide comprising an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide.
2. The system of embodiment 1, wherein the guide polynucleotide is a dead guide polynucleotide.
3. The system of embodiment 1 or embodiment 2, wherein the Cas polypeptide is a nuclease deficient Cas polypeptide.
4. The system of any one of embodiments 1-3, wherein the Cas polypeptide is fused to a transcriptional activation domain.
5. A system for simultaneously activating expression of a target nucleic acid and modifying a nucleotide sequence at a target site in a genome, the system comprising: (i) a Cas polypeptide, or a polynucleotide encoding the Cas polypeptide; (ii) a dead guide polynucleotide that mediates increased expression of a target nucleic acid, wherein the dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a guide polynucleotide that mediates sequence-specific cleavage at a target site in the genome.
6. A system for simultaneously activating expression of a first target nucleic acid and repressing expression of a second target nucleic acid, the system comprising: (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a second dead guide polynucleotide that mediates reduced expression of the second target nucleic acid.
7. A system for simultaneously activating expression of a first target nucleic acid, repressing expression of a second target nucleic acid, and modifying a nucleotide sequence at a target site in a genome, the system comprising: (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; (v) a second dead guide polynucleotide that mediates reduced expression of the second target nucleic acid; and (vi) a guide polynucleotide that mediates sequence-specific cleavage at a target site in the genome.
8. The system of any one of embodiments 1-7, wherein the Cas polypeptide is a Cas9 or a Cas12b.
9. The system of any one of embodiments 1-8, wherein the Cas polypeptide is a nuclease active Cas polypeptide.
10. The system of any one of embodiments 1-9, wherein the Cas polypeptide is not fused to a transcriptional activation domain.
11. The system of any one of embodiments 1-10, wherein the aptamer is an MS2 aptamer, and wherein the adapter domain comprises an MS2 bacteriophage coat protein (MCP).
12. The system of any one of embodiments 1-11, wherein the multimerized epitope comprises a GCN4 epitope.
13. The system of any one of embodiments 1-12, wherein the multimerized epitope comprises from about 2 copies of the GCN4 epitope to about 10 copies of the GCN4 epitope.
14. The system of any one of embodiments 1-13, wherein the multimerized epitope comprises 10 copies of the GCN4 epitope.
15. The system of any one of embodiments 1-14, wherein the affinity domain comprises scFv.
16. The system of any one of embodiments 1-15, wherein the transcriptional activation domain comprises a TAL Activation Domain (TAD), two repeats of TAD (2xTAD), 2xTAD-VP64, TV, VPR, or VP64.
17. The system of any one of embodiments 1-16, wherein the transcriptional activation domain comprises 2xTAD.
18. The system of any one of embodiments 1-17, wherein the Cas polypeptide is fused to a deaminase domain.
19. The system of any one of embodiments 1-18, wherein the components are located on one or more vectors.
20. A plant or a plant cell comprising the system of any one of embodiments 1-19.
21. A plant or plant cell comprising: (i) a Cas polypeptide, or a polynucleotide encoding the Cas polypeptide; (ii) a first dead guide polynucleotide that mediates increased expression of a first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; and (iv) a polypeptide a comprising an affinity domain and a transcriptional activation domain, wherein the affinity domain binds the multimerized epitope, or a polynucleotide encoding the polypeptide.
22. The plant or plant cell of embodiment 20 or embodiment 21 further comprising: a second dead guide polynucleotide that mediates reduced expression of a second target nucleic acid; and/or a guide polynucleotide that mediates sequence-specific cleavage at a target site in the genome of the plant cell.
23. The plant or plant cell of any one of embodiments 20-22, wherein the Cas polypeptide is a Cas9 or a Cas12b.
24. The plant or plant cell of any one of embodiments 20-23, wherein the Cas polypeptide is a nuclease active Cas polypeptide.
25. The plant or plant cell of any one of embodiments 20-24, wherein the Cas polypeptide is not fused to a transcriptional activation domain.
26. The plant or plant cell of any one of embodiments 20-25, wherein the aptamer is an MS2 aptamer, and wherein the adapter domain comprises an MS2 bacteriophage coat protein (MCP).
27. The plant or plant cell of any one of embodiments 20-26, wherein the multimerized epitope comprises a GCN4 epitope.
28. The plant or plant cell of any one of embodiments 20-27, wherein the multimerized epitope comprises from about 2 copies of the GCN4 epitope to about 10 copies of the GCN4 epitope.
29. The plant or plant cell of any one of embodiments 20-28, wherein the multimerized epitope comprises 10 copies of the GCN4 epitope.
30. The plant or plant cell of any one of embodiments 20-29, wherein the affinity domain comprises scFv.
31. The plant or plant cell of any one of embodiments 20-30, wherein the transcriptional activation domain comprises a TAL Activation Domain (TAD), two repeats of TAD (2xTAD), 2xTAD-VP64, TV, VPR, or VP64.
32. The plant or plant cell of any one of embodiments 20-31, wherein the transcriptional activation domain comprises 2xTAD.
33. The plant or plant cell of any one of embodiments 20-32, wherein the Cas polypeptide is fused to a deaminase domain.
34. A method for activating expression of a target nucleic acid in a plant cell, the method comprising: introducing in the plant cell (i) a Cas polypeptide, or a polynucleotide encoding the Cas polypeptide; (ii) a guide polynucleotide comprising an aptamer; (iii) a polypeptide comprising an adapter domain and a multimerized epitope, wherein the adapter domain binds the aptamer, or a polynucleotide encoding the polypeptide; (iv) a polypeptide a comprising an affinity domain and a transcriptional activation domain, wherein the affinity domain binds the multimerized epitope, or a polynucleotide encoding the polypeptide.
35. The method of embodiment 34, wherein the guide polynucleotide is a dead guide polynucleotide.
36. The method of embodiment 34 or embodiment 35, wherein the Cas polypeptide is a nuclease deficient Cas polypeptide.
37. The method of any one of embodiments 34-36, wherein the Cas polypeptide is fused to a transcriptional activation domain.
38. A method for simultaneously activating expression of a target nucleic acid and modifying a nucleotide sequence at a target site in a genome of a plant cell, the method comprising: introducing in the plant cell (i) a Cas polypeptide, or a polynucleotide encoding the Cas polypeptide; (ii) a dead guide polynucleotide that mediates increased expression of a target nucleic acid, wherein the dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a guide polynucleotide that mediates sequence-specific cleavage at a target site in the genome.
39. A method for simultaneously activating expression of a first target nucleic acid and repressing expression of a second target nucleic acid in a plant cell, the method comprising: introducing in the plant cell (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; and (v) a second dead guide polynucleotide that mediates reduced expression of the second target nucleic acid.
40. A method for simultaneously activating expression of a first target nucleic acid, repressing expression of a second target nucleic acid, and modifying a nucleotide sequence at a target site in a genome of a plant cell, the method comprising: introducing in the plant cell (i) a Cas polypeptide, or a polynucleotide encoding the Cas polypeptide; (ii) a first dead guide polynucleotide that mediates increased expression of the first target nucleic acid, wherein the first dead guide polynucleotide comprises an aptamer; (iii) a polypeptide comprising an adapter domain and a multimerized epitope, wherein the adapter domain binds the aptamer, or a polynucleotide encoding the polypeptide; (iv) a polypeptide comprising an affinity domain and a transcriptional activation domain, wherein the affinity domain binds the multimerized epitope, or a polynucleotide encoding the polypeptide; (v) a second dead guide polynucleotide that mediates reduced expression of the second target nucleic acid; and (vi) a guide polynucleotide that mediates sequence-specific cleavage at a target site in the genome.
41. The method of any one of embodiments 34-40, wherein the Cas polypeptide is a Cas9 or a Cas12b.
42. The method of any one of embodiments 34-41, wherein the Cas polypeptide is a nuclease active Cas polypeptide.
43. The method of any one of embodiments 34-42, wherein the Cas polypeptide is not fused to a transcriptional activation domain.
44. The method of any one of embodiments 34-43, wherein the aptamer is an MS2 aptamer, and wherein the adapter domain comprises an MS2 bacteriophage coat protein (MCP).
45. The method of any one of embodiments 34-44, wherein the multimerized epitope comprises a GCN4 epitope.
46. The method of any one of embodiments 34-45, wherein the multimerized epitope comprises from about 2 copies of the GCN4 epitope to about 10 copies of the GCN4 epitope.
47. The method of any one of embodiments 34-46, wherein the multimerized epitope comprises 10 copies of the GCN4 epitope.
48. The method of any one of embodiments 34-47, wherein the affinity domain comprises scFv.
49. The method of any one of embodiments 34-48, wherein the transcriptional activation domain comprises a TAL Activation Domain (TAD), two repeats of TAD (2xTAD), 2xTAD-VP64, TV, VPR, or VP64.
50. The method of any one of embodiments 34-49, wherein the transcriptional activation domain comprises 2xTAD.
51. The method of any one of embodiments 34-50, wherein the Cas polypeptide is fused to a deaminase domain.
52. The method of any one of embodiments 34-51, wherein the components are located on one or more vectors.
53. A vector comprising (i) a polynucleotide encoding a Cas polypeptide; (ii) a polynucleotide encoding a polypeptide comprising an adapter domain and a multimerized epitope, wherein the adapter domain is capable of binding an aptamer; and (iii) a polynucleotide encoding a polypeptide comprising an affinity domain and a transcriptional activation domain, wherein the affinity domain binds the epitope.
54. The vector of embodiment 53, wherein the Cas polypeptide is a Cas9 or a Cas12b.
55. The vector of embodiment 53 or embodiment 54, wherein the Cas polypeptide is a nuclease active Cas polypeptide
56. The vector of embodiment 53 or embodiment 54, wherein the Cas polypeptide is a nuclease deficient Cas polypeptide.
57. The vector of any one of embodiments 53-56, wherein the Cas polypeptide is fused to a transcriptional activation domain.
58. The vector of any one of embodiments 53-56, wherein the Cas polypeptide is not fused to a transcriptional activation domain.
59. The vector of any one of embodiments 53-58, wherein the adapter domain comprises an MS2 bacteriophage coat protein (MCP) capable of binding an MS2 aptamer.
60. The vector of any one of embodiments 53-59, wherein the multimerized epitope comprises a GCN4 epitope.
61. The vector of any one of embodiments 53-60, wherein the multimerized epitope comprises from about 2 copies of the GCN4 epitope to about 10 copies of the GCN4 epitope.
62. The vector of any one of embodiments 53-61, wherein the multimerized epitope comprises 10 copies of the GCN4 epitope.
63. The vector of any one of embodiments 53-62, wherein the affinity domain comprises scFv.
64. The vector of any one of embodiments 53-63, wherein the transcriptional activation domain comprises a TAL Activation Domain (TAD), two repeats of TAD (2xTAD), 2xTAD-VP64, TV, VPR, or VP64.
65. The vector of any one of embodiments 53-64, wherein the transcriptional activation domain comprises 2xTAD.
66. The vector of any one of embodiments 53-65, wherein the Cas polypeptide is fused to a deaminase domain.
67. The vector of any one of embodiments 53-66, wherein the vector comprises a nucleic acid sequence having at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% to the nucleic acid sequence of any of SEQ ID NOs: 37-47.
68. The vector of any one of embodiments 53-67, wherein the vector comprises the nucleic acid sequence of any of SEQ ID NOs: 37-47.

### EXAMPLES

### Example 1: Development of the CRISPR-Act3.0 System

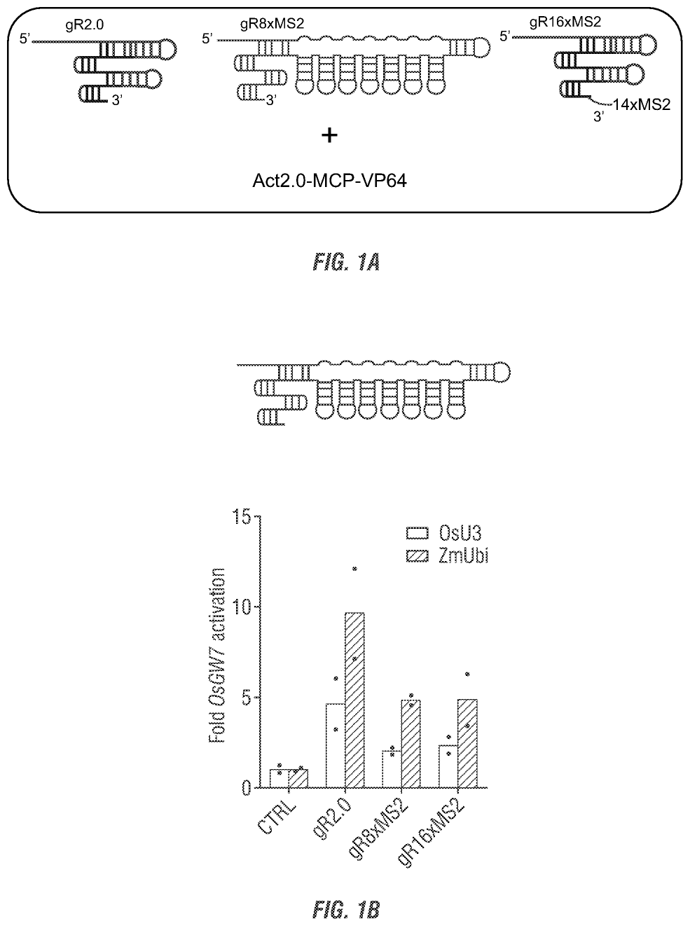

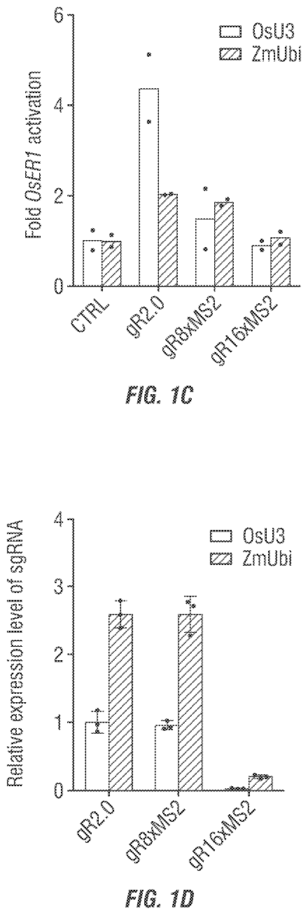

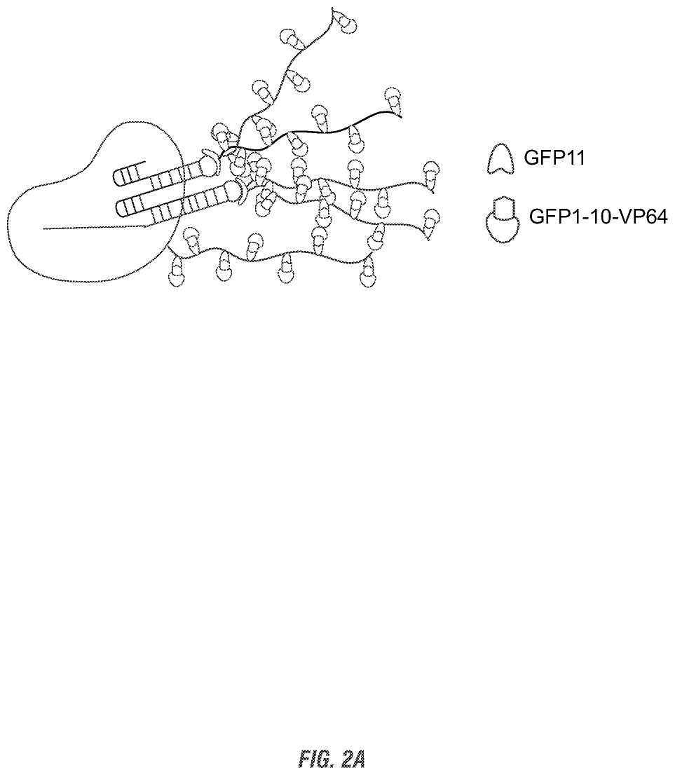

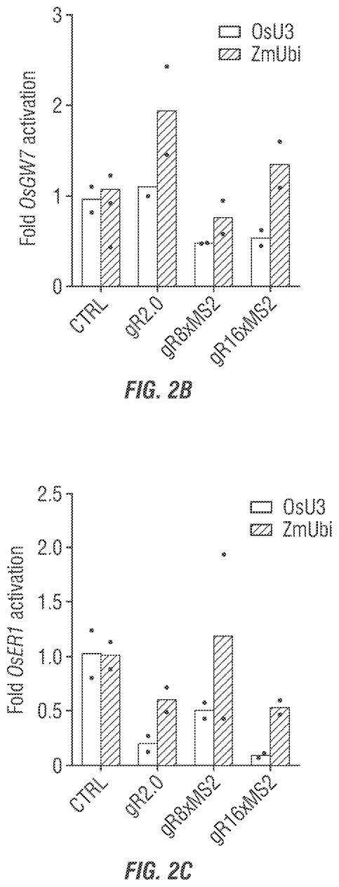

Our previous CRISPR-Act2.0 system utilized an engineered gRNA2.0 (gR2.0) scaffold that contains two MS2 RNA aptamers for recruiting activator VP64 through the MS2-MCP interaction. We reasoned that by installing more MS2 aptamers into the single guide RNA (sgRNA) scaffold the system could recruit more VP64 that might lead to improved gene activation. We adopted sgRNA scaffolds containing 8xMS2 (gR8xMS2) and 16xMS2 (gR16xMS2), as both scaffolds were previously demonstrated to recruit many copies of fluorescent proteins for live cell imaging of mammalian cells (FIG. 1A). We compared these two new VP64-recruiting systems coupled with CRISPR-Act2.0 for gene activation in rice protoplasts. Two independent genes, OsGW7 and OsER1, were targeted for activation. We also compared OsU3 (rice U3; a Pol III promoter) and ZmUbi (maize ubiquitin 1; a Pol II promoter) for expressing the sgRNAs. To guide proper sgRNA maturation in the ZmUbi promoter system, the tRNA processing system was used. To our surprise, neither gR8xMS2 nor gR16xMS2 showed higher gene activation levels than the CRISPR-Act2.0 system, based on quantitative reverse transcription PCR (qRT-PCR) analysis (FIG. 1B-C). We found that the guide RNA level of gR16xMS2 was much lower than those of gR2.0 and gR8xMS2 with either promoter (FIG. 1D), indicating the instability of gR16xMS2 might be the bottleneck for its activation efficiency. In addition, compared with OsU3, ZmUbi produced higher guide RNA levels (FIG. 1D). To recruit more VP64 with these three sgRNA scaffolds, we modified a split GFP system. In our system, a deactivated plant codon-optimized Cas9 (dpcoCas9) is fused to seven tandemly arrayed GFP11 peptides and co-expressed with a GFP1-10-VP64 fusion protein (FIG. 2A). Upon GFP reconstitution, a single dCas9/sgRNA complex is expected to recruit many VP64 to a target site. Testing this strategy in rice protoplasts, however, did not yield gene activation for either OsGW7 or OsER1 (FIG. 2B-C).

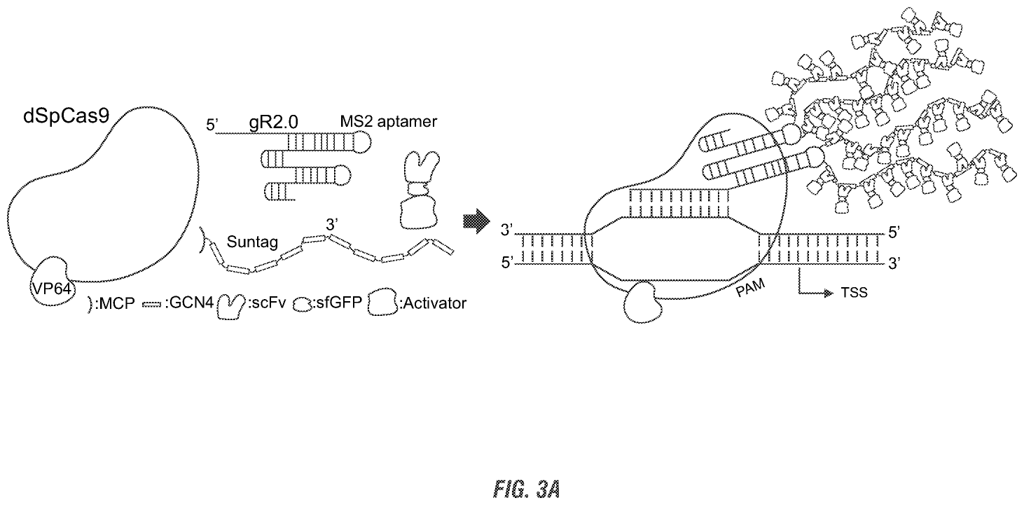

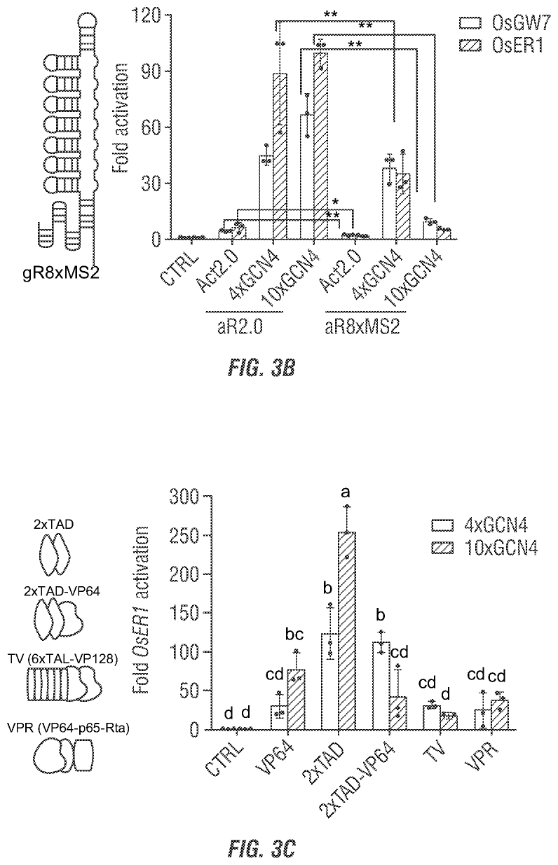

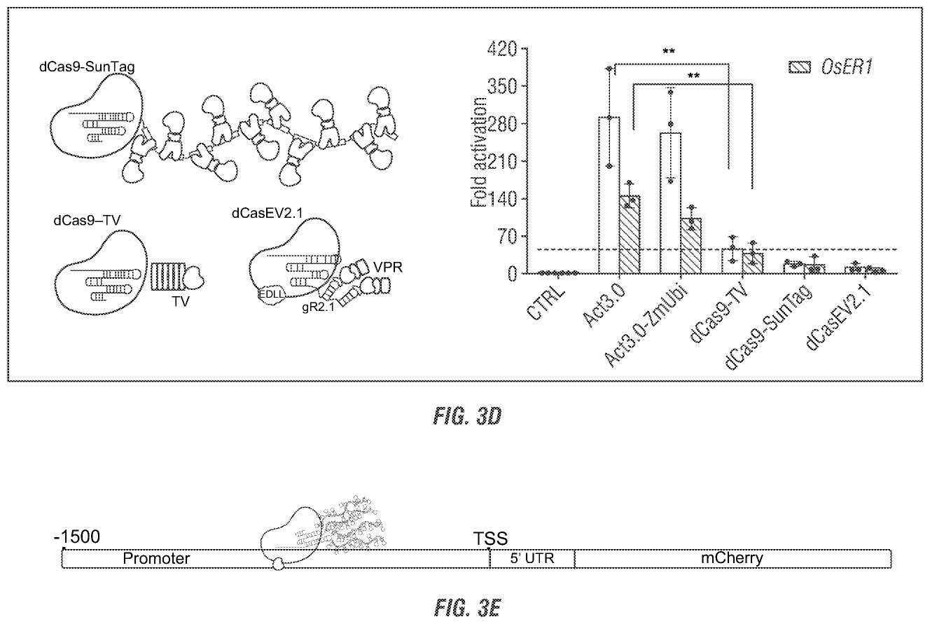

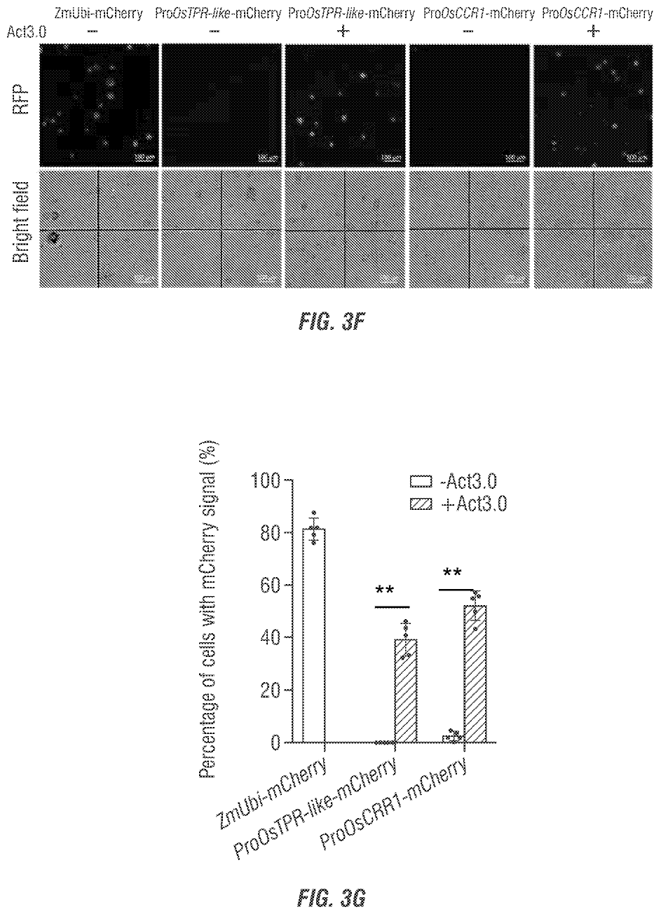

These earlier attempts suggested that some strategies which successfully recruit fluorescent proteins for DNA imaging do not result in gene activation through recruitment of transcription activators such as VP64. This could be due to the complex process of gene activation as it requires further recruitment of transcription machinery based on the activators. The SunTag system has been previously established for gene activation in both human cells and plants. In the SunTag system, the tandemly arrayed GCN4 epitopes are directly fused to the C-terminus dCas9 to recruit VP64 through a single-chain antibody scFv. We hypothesized that coupling the SunTag system with the MS2-MCP interaction would recruit more VP64 (FIG. 3A). To test this strategy, we compared both gR2.0 and gR8xMS2 scaffolds with two different lengths of SunTag (4xGCN4 and 10xGCN4). We assessed these configurations in rice protoplasts by activating OsGW7 and OsER1. With the gR2.0 scaffold, both 4xGCN4 and 10xGCN4 tags resulted in pronounced gene activation at both targets, which was 10-fold higher than the level induced by the CRISPR-Act2.0 system (FIG. 3B). The gR8xMS2 scaffold also generated significant activation, though less potent than the gR2.0 scaffold (FIG. 3B). These results reinforced the notion that more MS2 aptamers did not necessarily translate into higher gene activation.

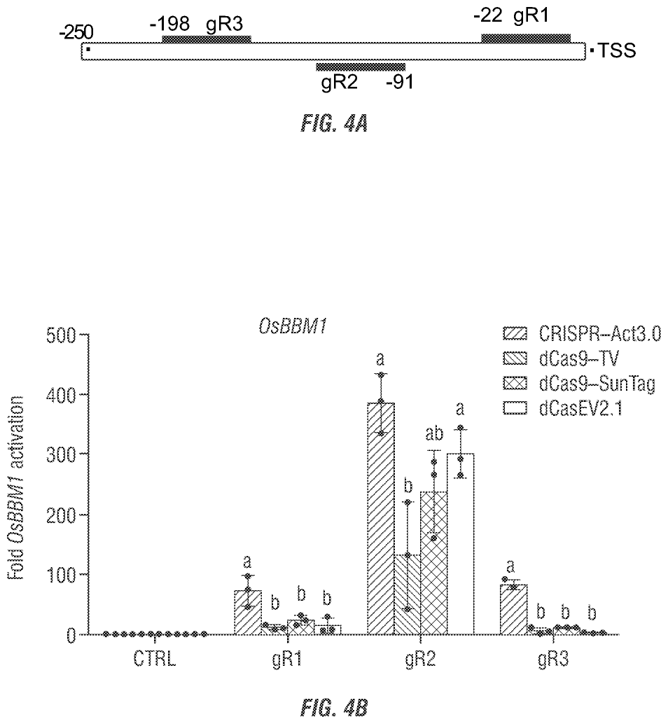

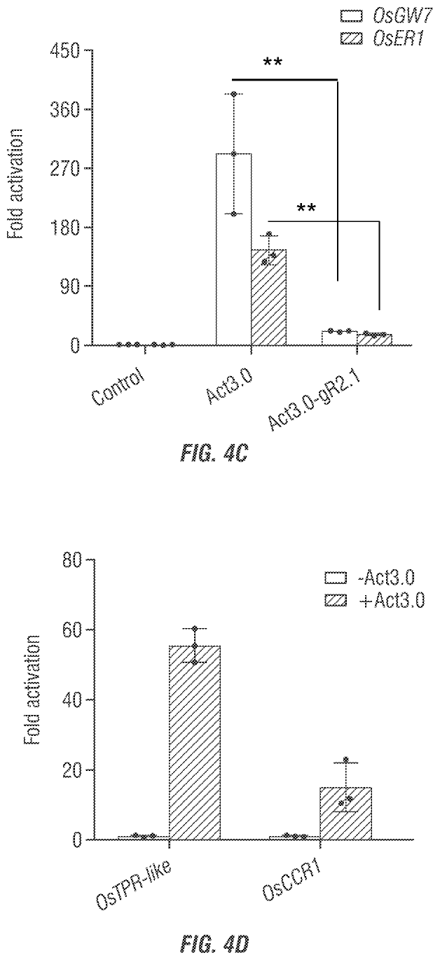

Encouraged by the success in combining the SunTag system with the MS2 system, we next developed two new activators 2xTAD (TAL Activation Domain) and 2xTAD-VP64 and compared them with previously reported activators VP64, TV and VPR to test this platform by targeting OsER1 in rice protoplasts (FIG. 3C). With the 4xGCN4 SunTag, the systems with 2xTAD and 2xTAD-VP64 activators showed highest gene activation (>100 fold) (FIG. 3C). With the 10xGCN4 SunTag, the 2xTAD activator system resulted in the highest activation of the target gene, ˜250-fold (FIG. 3C) This result pointed to a highly efficient gene activation system that combines dCas9-VP64, gR2.0 scaffold, 10xGCN4 SunTag, and the newly developed 2xTAD activator. We consider this new system as a 3rd generation CRISPRa system and named it CRISPR-Act3.0. To benchmark CRISPR-Act3.0, we compared it with three additional 2^{nd} generation CRISPRa systems previously developed by other research groups, including dCas9-SunTag, dCas9-TV, and dCasEV2.1 (FIG. 3D). To ensure close comparison, the same vector backbones and promoters were used. By targeting the same two genes (OsGW7 and OsER1) with the same sgRNAs, we found dCas9-TV resulted in ˜40-fold activation of both genes, an activation level comparable to the previous report with using dCas9-TV at activating these genes. With this level of gene activation, the dCas9-TV system outperformed the dCas9-SunTag and dCasEV2.1 systems (FIG. 3D). Strikingly, CRISPR-Act3.0 generated four- to six-times stronger activation than dCas9-TV at both target genes, with over 250-fold for OsGW7 and over 100-fold for OsER1, regardless of the promoter (OsU3 or ZmUbi) used to drive the single sgRNA expression (FIG. 3D). To further benchmark CRISPR-Act3.0, we targeted a third gene in rice, OsBBM1, whose overexpression in egg cells was recently shown to help asexual propagation of rice seeds. To rule out that superior performance of CRISPR-Act3.0 is position-dependent, we targeted three distinct positions in the OsBBM1 promoter (FIG. 4A). We found that the CRISPR-Act3.0 resulted in significantly higher activation efficiency at two target sites and considerably improved activation at one target site compared to the other three activation systems: 6- to 24-fold higher at two target sites and 1.3- to 2.9- fold higher at the third site (FIG. 4B). Since both gR2.0 and gRNA2.1 scaffolds contain two MS2 stem loops, albeit different positions, we compared them and found gRNA2.1 worked poorly in the CRISPR-Act3.0 configuration for gene activation compared to gRNA2.0 (FIG. 4C). Together, our work established gR2.0 based CRISPR-Act3.0 as a 3^{rd} generation CRISPRa system that is much more potent than earlier systems based on our assays in rice protoplasts.

We next sought to visualize the CRISPR-Act3.0-mediated activation by using a mCherry reporter system. Two randomly selected promoters ProOsTPR-like and ProOsCCR1 were used to drive mCherry expression, generating two corresponding mCherry reporter constructs (FIG. 3E). As a positive control, mCherry was driven by the strong ZmUbi promoter. Each promoter except the positive control was targeted by one sgRNA to evaluate the robustness of CRISPR-Act3.0. Notably, co-transformation of rice protoplasts with the CRISPR-Act3.0 construct and the reporter construct in both cases resulted in red fluorescent cells indicative of strong mCherry expression; in contrast, such signals were absent without the use of either sgRNA (FIG. 3F). Further quantification showed that 80% of cells were mCherry-positive from the ZmUbi::mCherry positive control. About 40% and 50% of cells co-transformed with the CRISPR-Act3.0 and the mCherry report constructs were mCherry-positive, respectively (FIG. 3G), suggesting CRISPR-Act3.0-induced activation potency can be indicated by fluorescence intensity. In addition, CRISPR-Act3.0 activated the transcription of the endogenous genes OsTPR-like and OsCCR1 in rice with ˜60-fold and ˜20-fold activation, respectively (FIG. 4D). Taken together, these data demonstrated robust gene activation by CRISPR-Act3.0 with a single sgRNA.

### Example 2: Multiplexed Gene Activation in Rice

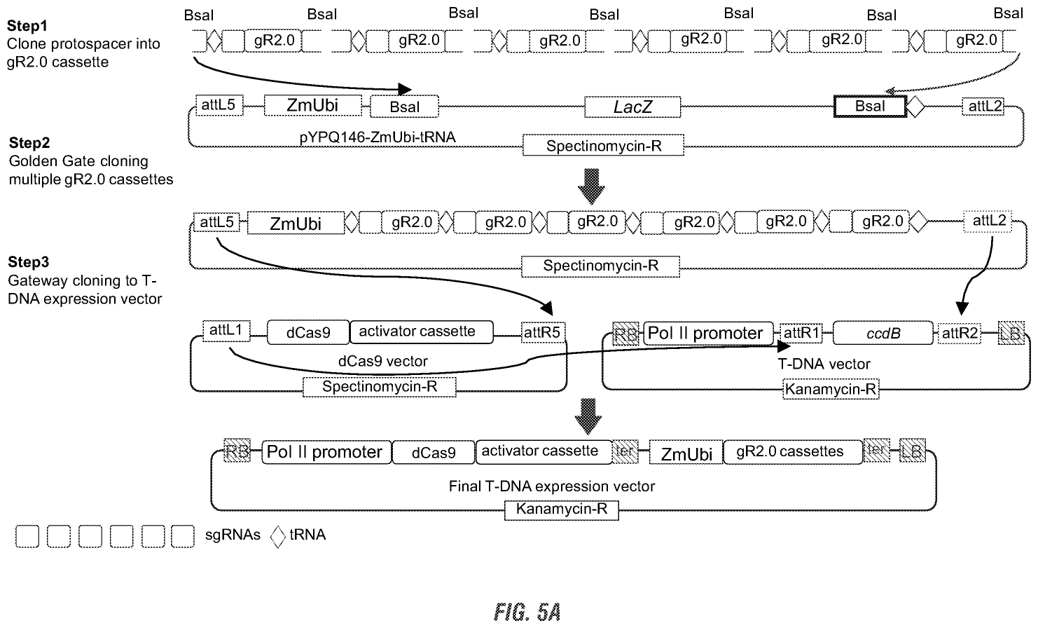

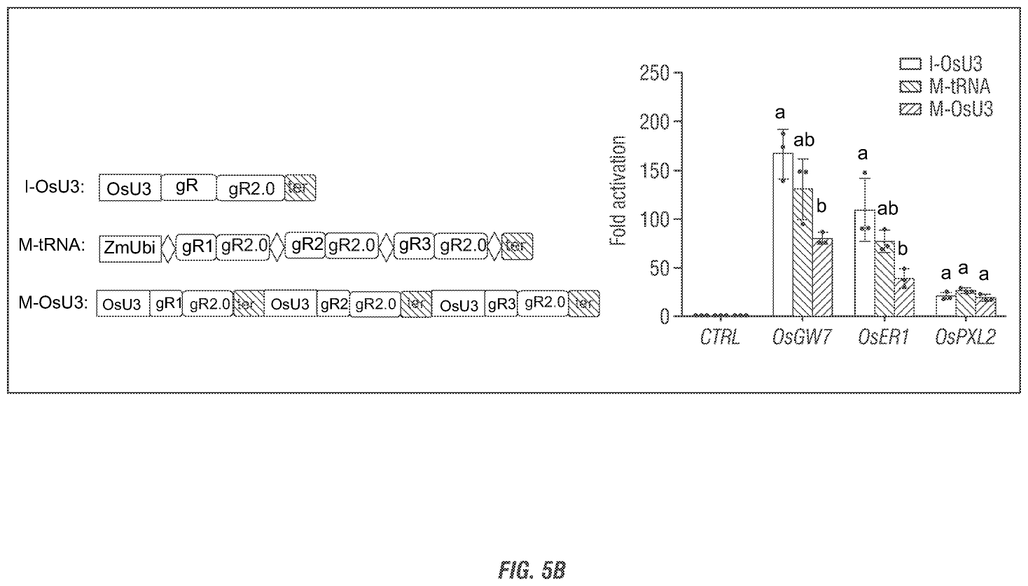

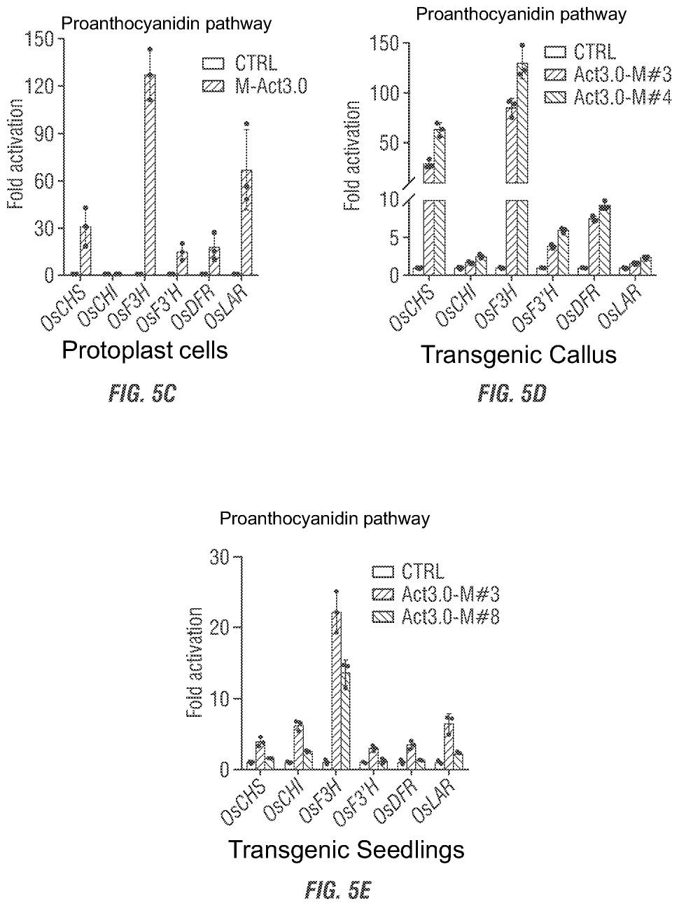

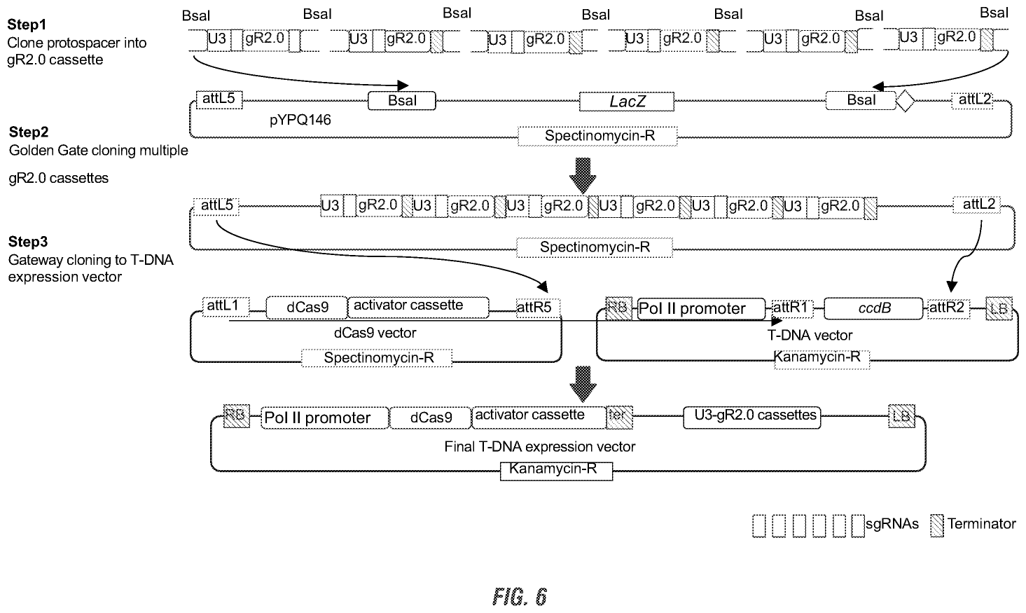

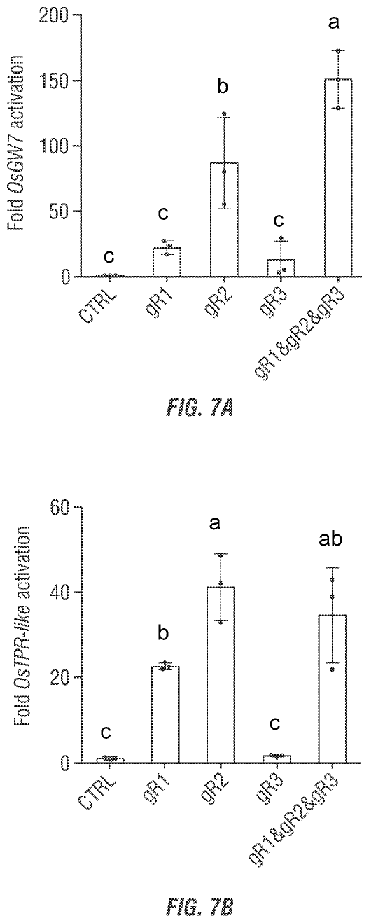

The tRNA-based processing system is highly compact and efficient for multiplexing sgRNAs in plants, yeast, Drosophila, and human cells. To enable efficient multiplexed gene activation in rice, we developed a streamlined cloning system for one-step assembly of up to six tRNA-gRNA2.0 cassettes (FIG. 5A) or U3-gRNA2.0 cassettes (based on a conventional gRNA2.0 system) (FIG. 6). One Pol II promoter ZmUbi was employed to drive all tRNA-gRNA2.0 cassettes expression, and in contrast, one U3 promoter was used for each individual U3-gRNA2.0 cassette expression (FIG. 5A and FIG. 6). To compare this multiplexed tRNA-gRNA2.0 (M-tRNA) system with the conventional multiplexed U3-gRNA2.0 (M-U3) system where sgRNAs were expressed in independent transcription units, we targeted three genes (OsGW7, OsER1 and OsPXL2) in rice for simultaneous activation (FIG. 5B). These two multiplex constructs, M-tRNA and M-OsU3, were compared with individual gene activation constructs (I-OsU3). At the three target genes, M-tRNA resulted in comparable levels of fold activation with I-OsU3 and improved activation than M-OsU3 for multiplexed gene activation (FIG. 5B). We next compared single sgRNA and multiplexed sgRNAs for gene activation at two independent loci. At OsGW7, multiplexing three gRNAs generated higher gene activation than single gRNAs alone (FIG. 7A). At OsTPR-like, multiplexing three sgRNAs generated a similar level of gene activation to the best performing single sgRNA (FIG. 7B). These data suggest multiplexing a few sgRNAs represents a safe strategy to achieve robust gene activation, consistent with earlier observations in plants.

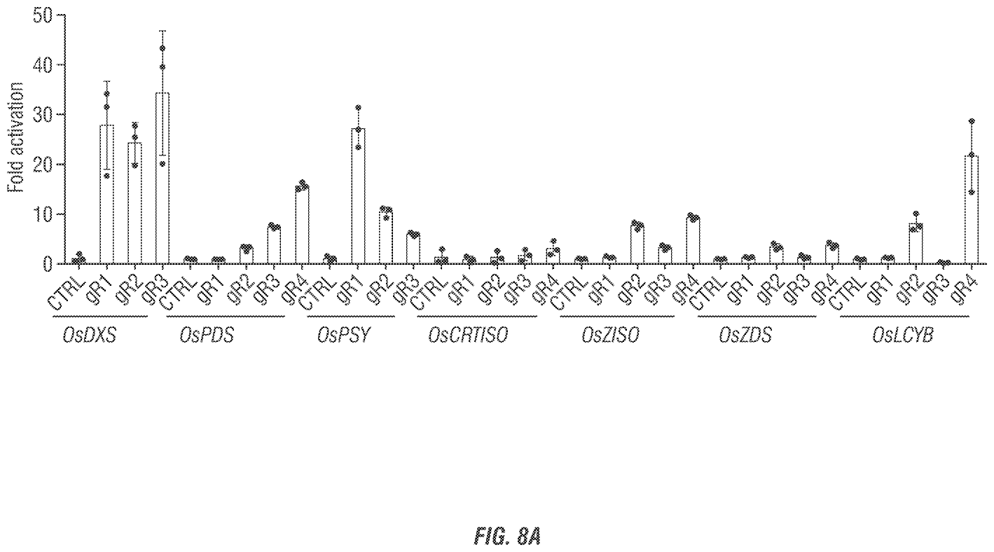

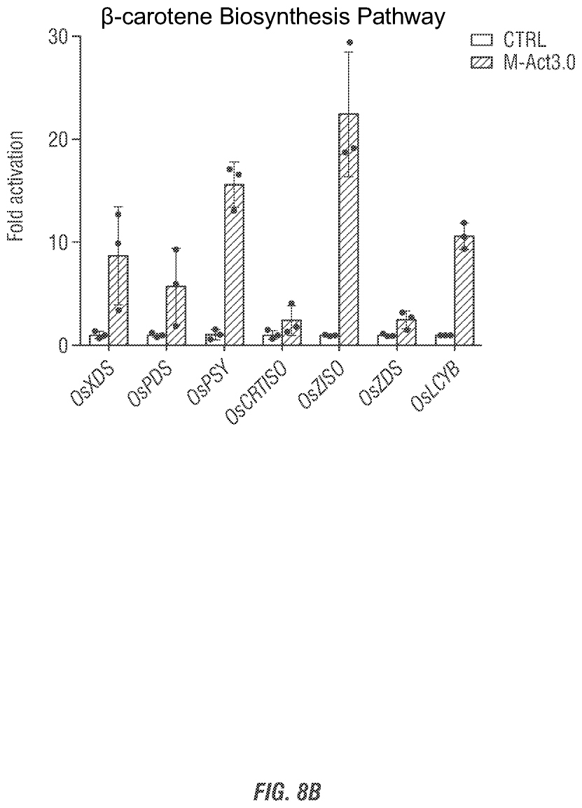

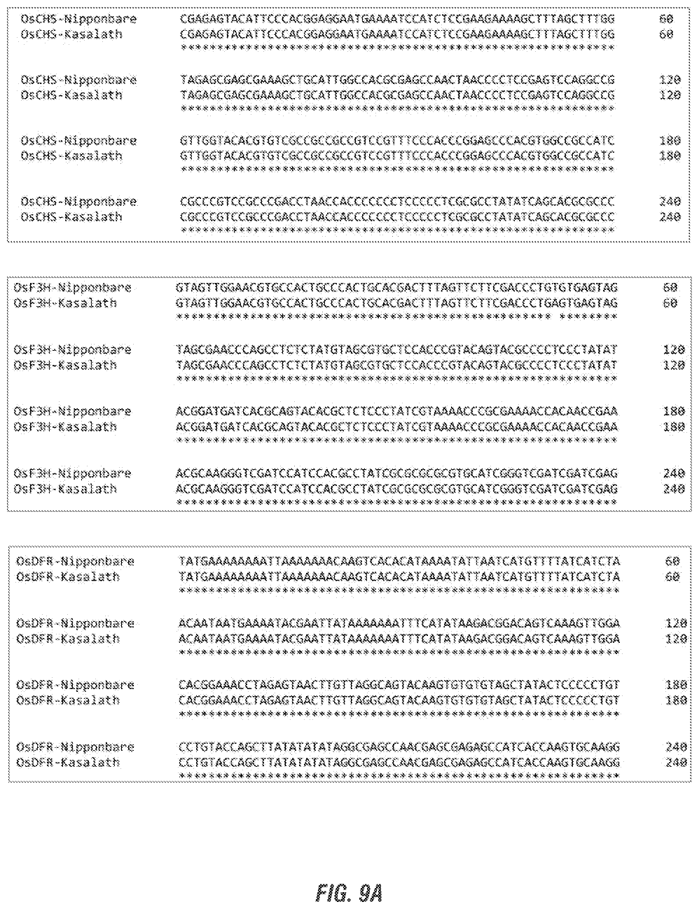

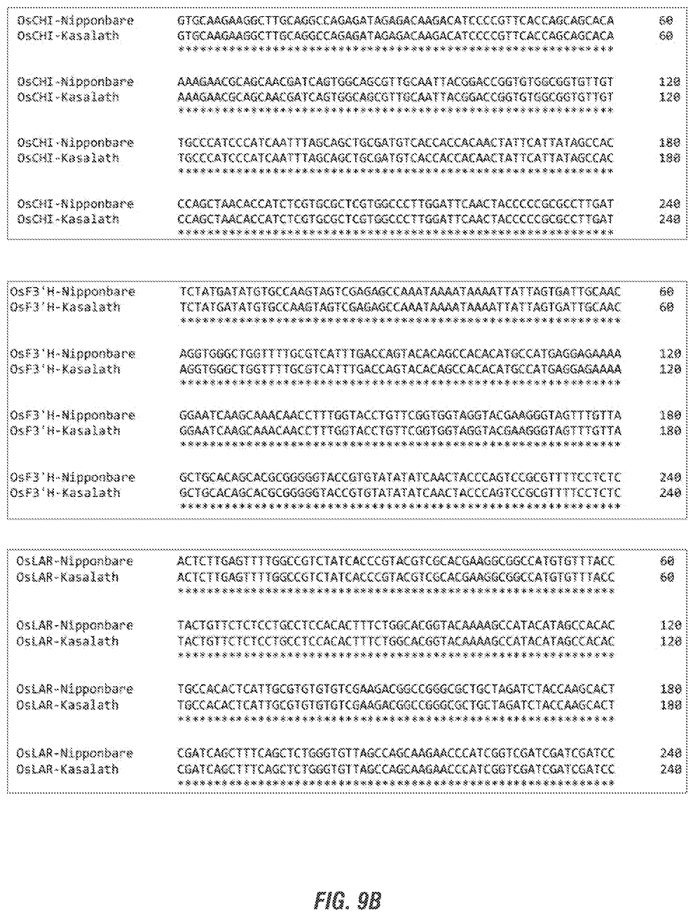

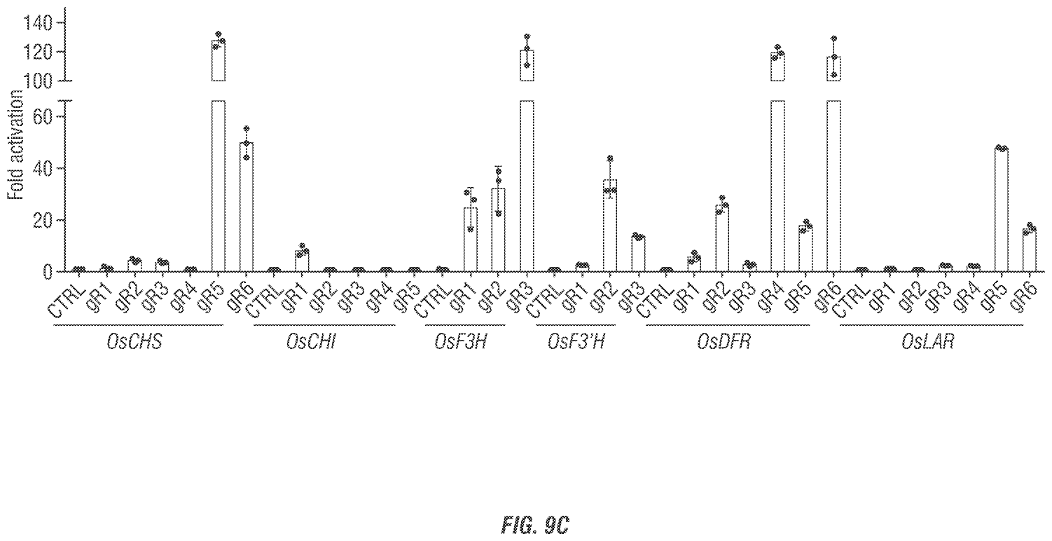

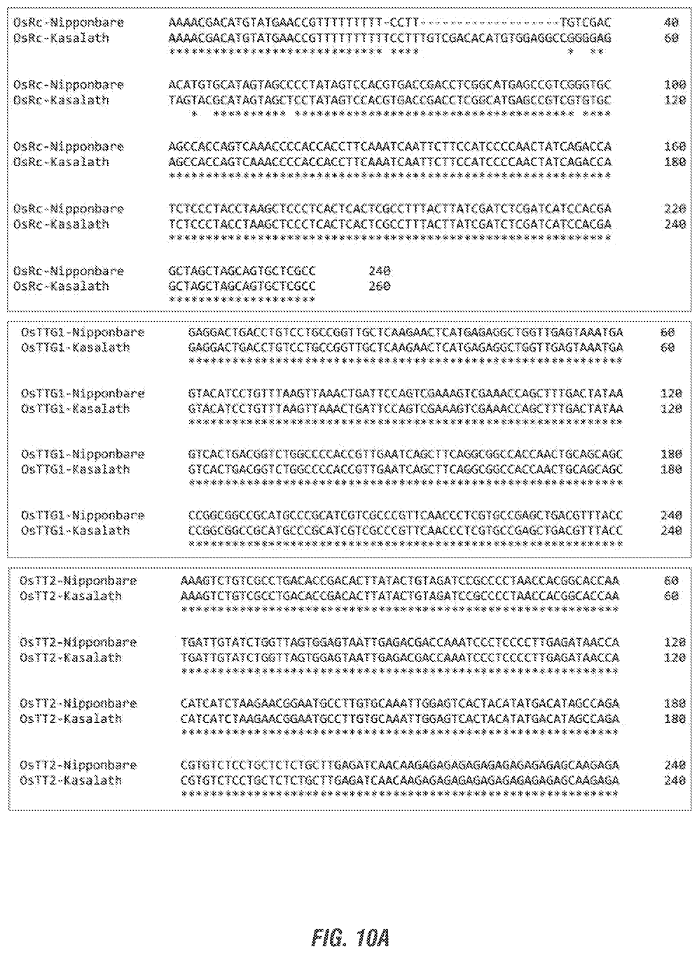

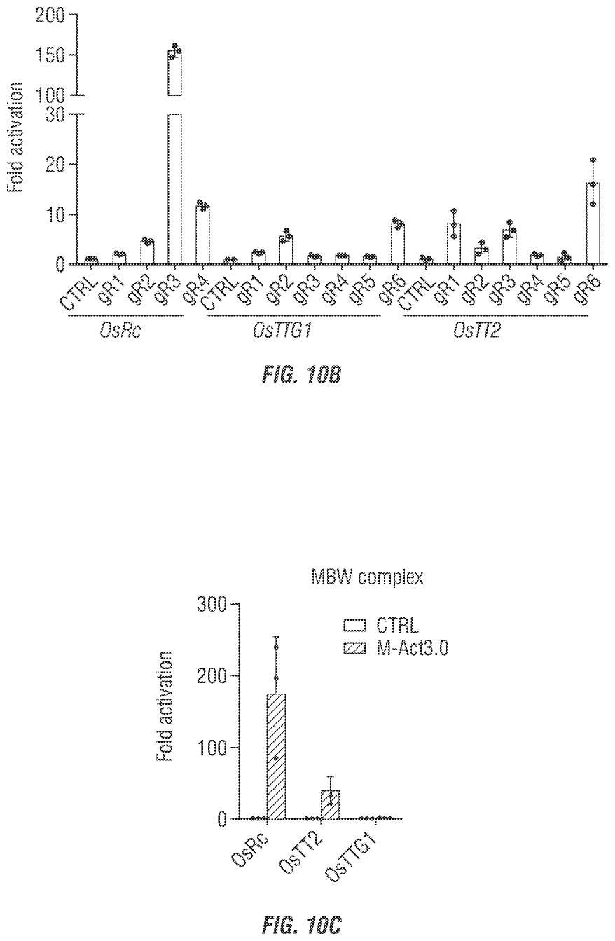

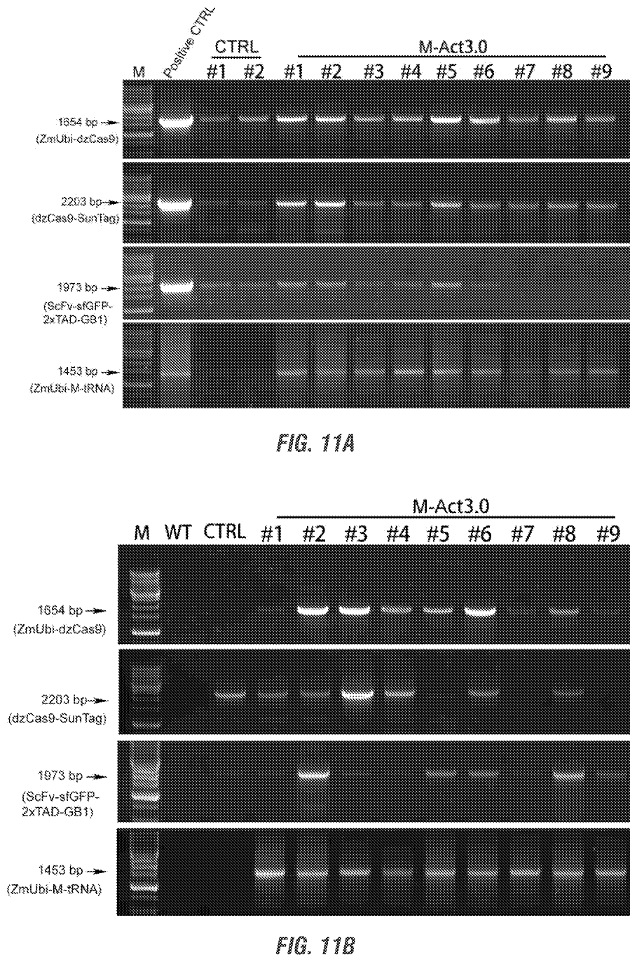

However, we also found that highly efficient singular sgRNAs could be identified using a protoplast-based prescreen process. In most cases, the activation level with a single sgRNA would be strong enough for the target gene, which reserves much room for multiplexing many genes as only one sgRNA is used for one gene. To demonstrate this one sgRNA for one gene strategy, we sought to apply CRISPR-Act3.0 to target metabolic pathway genes with the M-tRNA system. In a first demonstration, we targeted seven enzyme-encoding genes in the β-carotene pathway in rice. For each gene, three to four sgRNAs were tested in rice protoplasts in the prescreen step. Prescreen data in rice protoplasts showed four of seven genes could be activated 10-fold or higher (FIG. 8A). Then, we picked the best performing sgRNAs for each target gene and assembled them into one M-Act3.0 vector based on M-tRNA system according to a higher order assembly method. Gene activation of up to 20-fold was found for all seven target genes using the M-Act3.0 vector in rice protoplasts (FIG. 8B). In a second demonstration, we targeted six enzyme-encoding genes in the proanthocyanidin pathway in the indica rice variety Kasalath (FIG. 9A). For each gene, three to six sgRNAs were tested in rice protoplasts in the prescreen step, and in all cases except OsCHI at least one sgRNA could be identified with >30-fold gene activation levels (FIG. 9C). Stacking the six high-activity sgRNAs with the M-tRNA system (FIG. 5A) led to pronounced simultaneous gene activation for five out of six target genes (FIG. 5C). We also targeted three regulatory genes (OsRc, OsTTG1 and OsTT2) in the proanthocyanidin pathway (FIG. 10A). Two sgRNAs were employed for each target gene. These regulatory genes were individually activated by M-Act3.0 and two of them were activated by 40-fold simultaneously with the M-tRNA system (FIG. 10B-C).

Furthermore, we used Agrobacterium-mediated transformation to introduce a M-Act3.0 vector targeting the six enzyme-encoding genes in the proanthocyanidin pathway and the no-sgRNA control vector into the indica rice variety Kasalath. The M-Act3.0 system resulted in a similar activation pattern for all target genes except OsLAR in both rice protoplast cells and transgenic callus (FIG. 5C-D and FIG. 11A), and four out of six target genes were activated by 5- to 140-fold (FIG. 5D). However, only OsF 3H had a significant activation of ˜20-fold in M-Act3.0 transgenic seedlings (leaves) (FIG. 5E and FIG. 11B), and other five target genes could only be activated two- to eight-fold (FIG. 5E). In addition, no significant difference in phenotype in both callus and seedlings was observed between the M-Act3.0 and CTRL transgenic lines. Taken together, we have developed a multiplexed CRISPR-Act3.0 system and demonstrated its use for simultaneous activation of many genes in the agriculturally relevant crop, rice. Our data also suggest that the potency of CRISPRa is shaped by the endogenous gene regulatory mechanisms which may vary among genes and pathways.

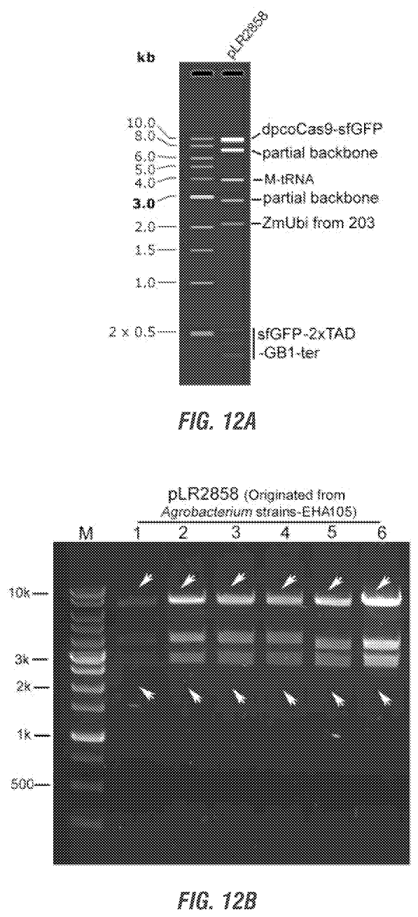

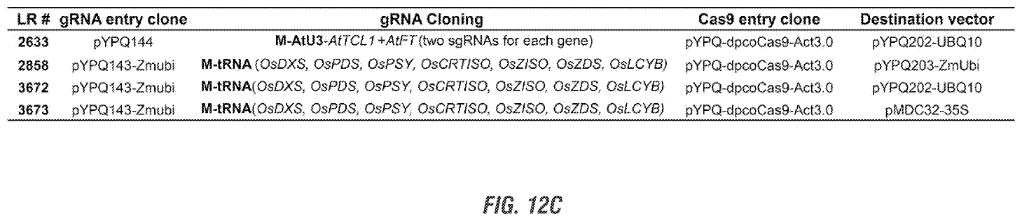

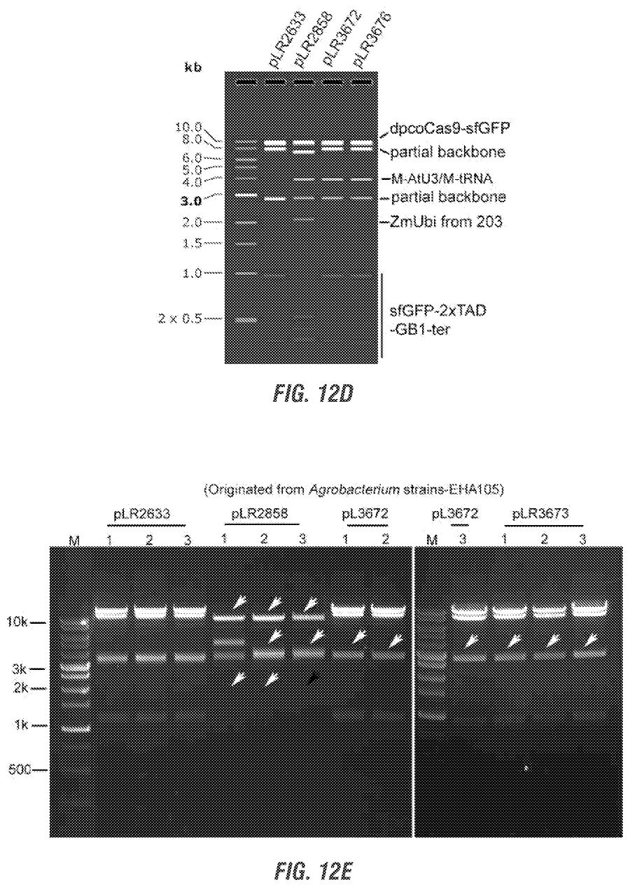

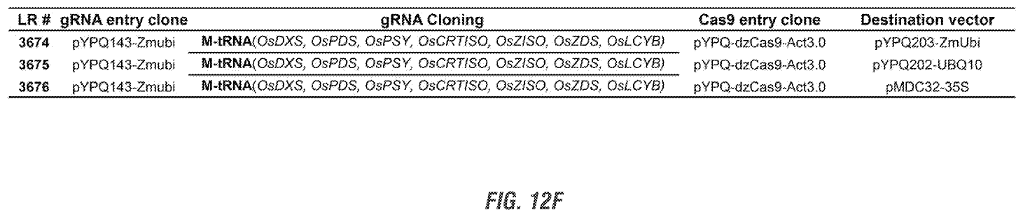

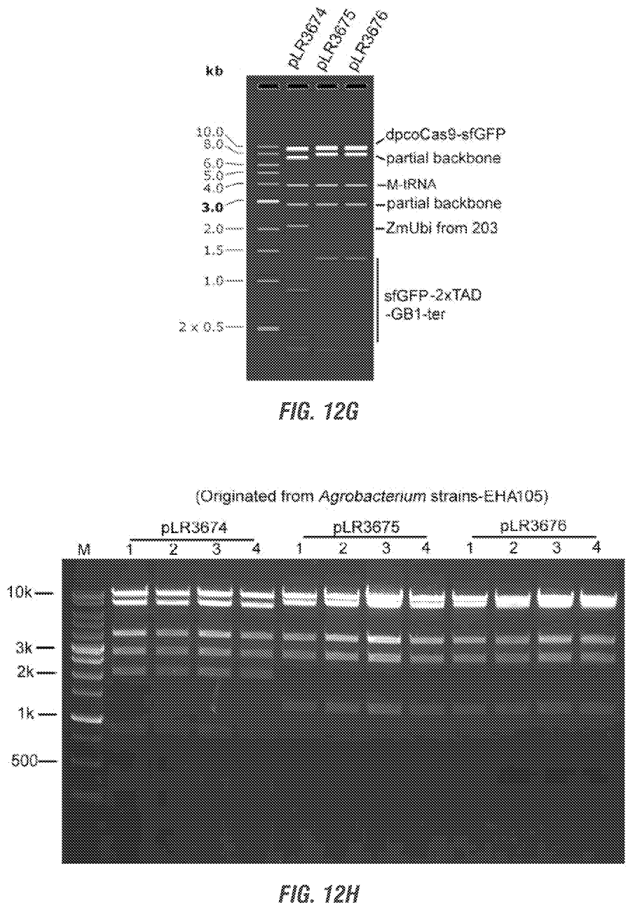

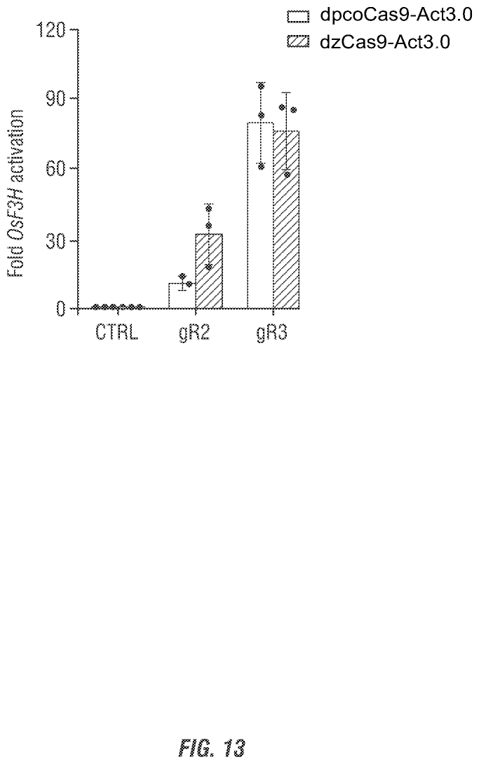

It is worth noting that the final T-DNA vector expressing dpcoCas9-Act3.0 and M-tRNA components could cause DNA rearrangements in A. tumefaciens EHA105 despite that different promoters (ZmUbi, UBQ10 (ubiquitin-10) or a cauliflower mosaic virus 35S) were used to drive the dpcoCas9 expression (FIG. 12A-E). However, such DNA rearrangements had not been found in the combination of dpcoCas9-Act3.0 and M-U3 systems (FIG. 12C-E). In contrast, we found that dzCas9 (a maize codon-optimized dSpCas9) based CRISPR-Act3.0 did not cause any DNA rearrangement in the plasmids in A. tumefaciens (FIG. 12F-H). The dzCas9 based CRISPR-Act3.0 system induced a comparable activation efficiency with the dpcoCas9 based CRISPR-Act3.0 system (FIG. 13), consistent with previous reports that both pcoCas9 and zCas9 proteins were efficient for genome editing.

### Example 3: Multiplexed Gene Activation in Dicot Plants

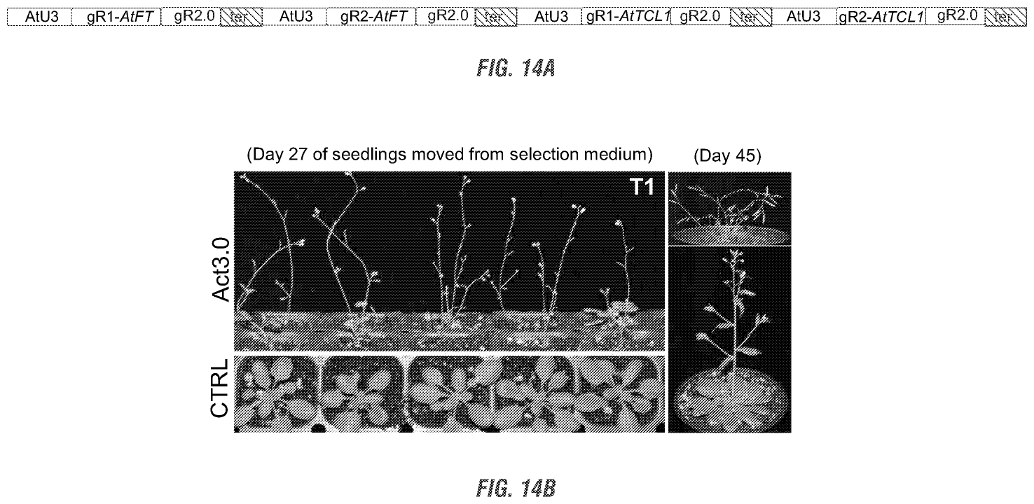

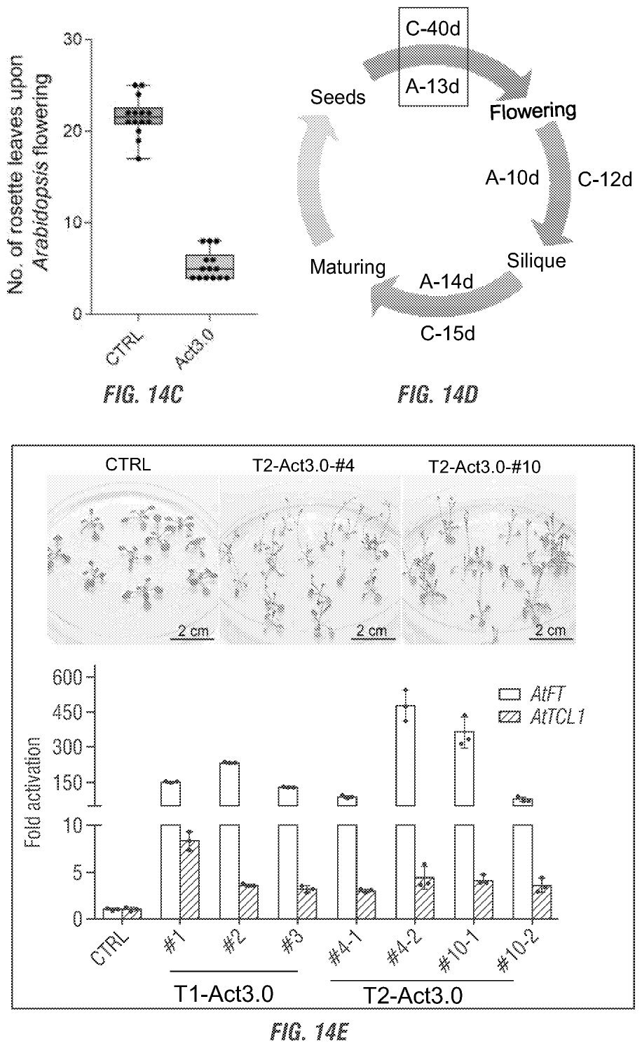

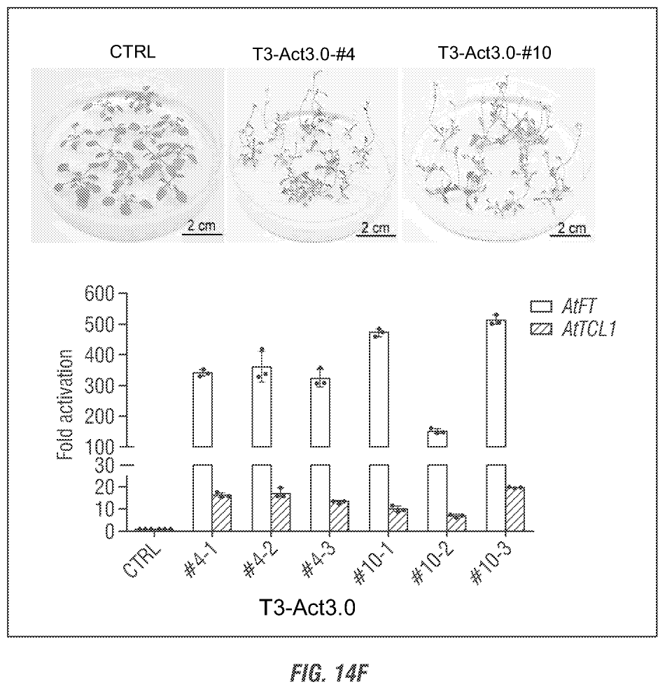

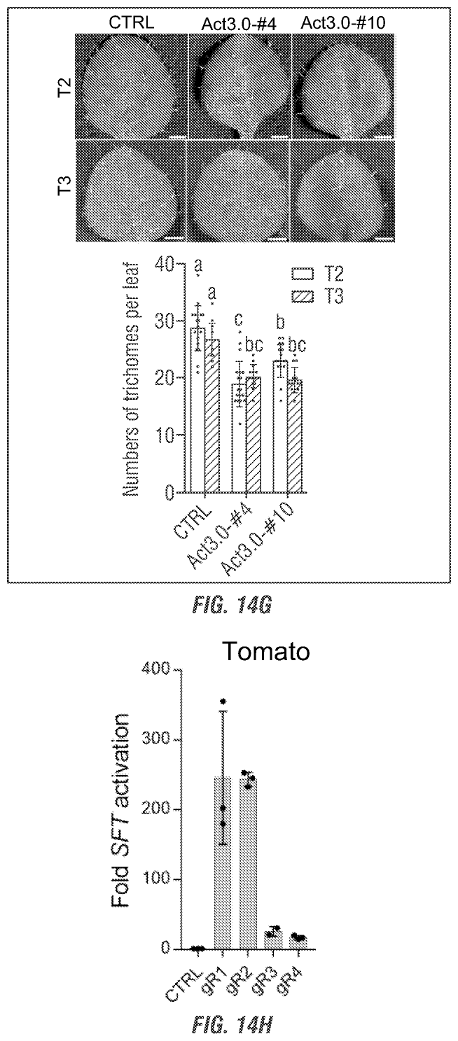

To assess CRISPR-Act3.0 in dicot plants, we simultaneously targeted two genes, AtFT (regulating flowering) and AtTCL1 (regulating trichome development), in the model plant Arabidopsis by the dpcoCas9 based CRISPR-Act3.0 system. Each gene was targeted with two sgRNAs and the four corresponding sgRNAs were assembled based on the streamlined cloning system (FIG. 14A and FIG. 6). All T1 transgenic plants clearly displayed early flowering phenotype (FIG. 14B), an anticipated phenotype for robust AtFT overexpression. The phenotype was further quantified by counting the number of rosette leaves upon flowering. The control plants showed about four times more rosettes leaves than the AtFT overexpression lines (FIG. 14C). A plant life cycle analysis showed transgenic plants on average reduced their seed-to-seed life cycle by ˜30 days compared to the no-sgRNA transgenic control plants (FIG. 14D). The expression levels of AtFT and AtTCL1 were activated by 130- to 240-fold and three- to eight-fold, respectively, in early flowering T1 plants (FIG. 14E). It is worth noting that relatively low levels of gene activation for AtTCL1 could be due to the lack of prescreening sgRNA activities. We further examined the elevated expression levels of AtFT and AtTCL1 as well as the resulting early flowering and reduced trichome development in T2 and T3 generations. A high percentage of early flowering phenotype (˜85%) was identified in both T2 and T3 populations of Act3.0-#4 and -#10 lines (FIG. 14E-F). In addition, the numbers of trichomes per leaf of Act3.0-#4 and -#10 lines were found significantly decreased in both T2 and T3 populations (FIG. 14G). Consistent with the phenotypes, similar levels of gene activation for both AtFT (80- to 500-fold) and AtTCL1 (three- to 20-fold) were found in both T2 and T3 populations (FIG. 14E-F). These results suggest that the CRISPR-Act3.0 is a robust gene activation tool in a dicot plant species and multiplexed CRISPR-Act3.0-mediated modifications of phenotypes can be stably transmitted across multiple generations. Translation of the success in CRISPR-Act3.0 mediated endogenous FT activation in Arabidopsis into crops would have transformative impacts in accelerating crop breeding.

Since zCas9 resulted high efficiency genome editing in dicot plants such as Arabidopsis and carrot, the dzCas9-Act3.0 system presumably should work well for gene activation in dicot plants. We tested dzCas9-Act3.0 in tomato. Four different sgRNAs (gR1 to gR4) were designed to target the promoter of the SFT gene in tomato. Based on a protoplast assay, gR1 and gR2 each resulted in 240-fold transcription activation, while gR3 and gR4 generated —30-fold and 20-fold transcription activation, respectively (FIG. 14H). The data suggest dzCas9-Act3.0 is very potent in tomato and the levels of target gene activation are determined by the sgRNAs and their target positions.

### Example 4: Design Rules for Efficient sgRNAs in CRISPR-Act3.0 Applications

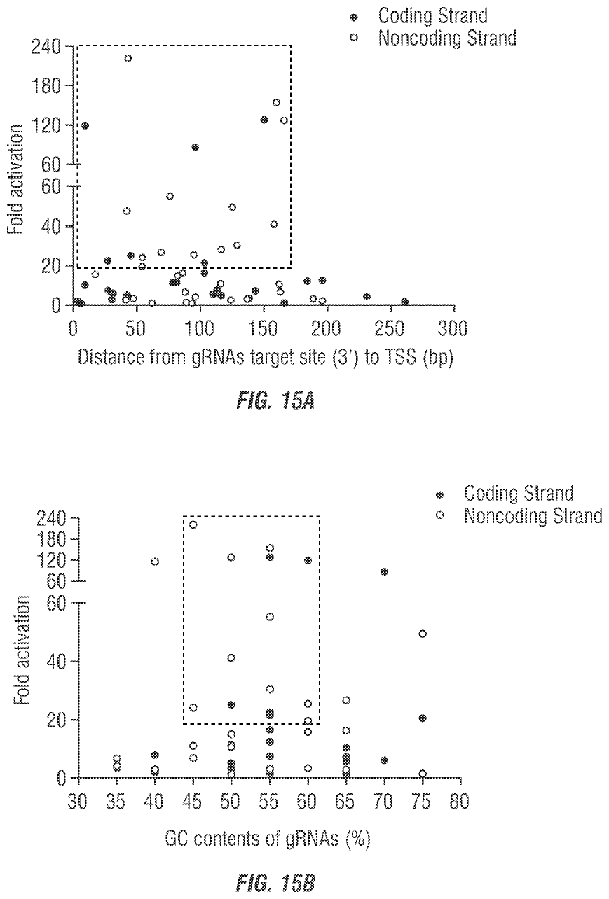

Our work here, along with earlier studies in plants, has shown that gene activation efficiency varies among different sgRNAs for the same target gene. When designing sgRNAs, we had already focused on the most effective promoter region, which is 0 bp to −250 bp from the transcription start site (TSS), according to earlier studies in humans. To provide further guidance in sgRNA design for implementing CRISPR-Act3.0 in plants, we investigated the protoplast-based gene activation data from 56 sgRNAs targeting the −3 bp to −261 bp region from the TSS of 16 genes in rice. We found that most sgRNAs were effective in the 0 bp to −200 bp region from the TSS (FIG. 15A). Interestingly, sgRNAs targeting the noncoding strand of DNA were overrepresented (13/19; sgRNAs targeting the noncoding strand/total sgRNAs, p<0.05, two-tailed binomial probability test) among these active sgRNAs with the threshold of 20-fold activation (FIG. 15A), suggesting sgRNAs targeting the noncoding strand DNA are preferred to achieve higher activation activity. Further analyses showed that sgRNAs with GC content between 45% to 60% resulted in higher frequency of robust gene activation (average activation 34.8-fold within optimum range, 12.4-fold outside optimum range, p<0.05, Kruskal-Wallis test) (FIG. 15B). This trend matches prior reports that sgRNAs with extreme GC contents are less active for gene editing. We note that these design guidelines, although useful for initial implementation of CRISPR-Act3.0, require further corroboration and investigation with larger data sets, ideally from different plant species.

### Example 5: Expanding the Targeting Scope of CRISPR-Act3.0

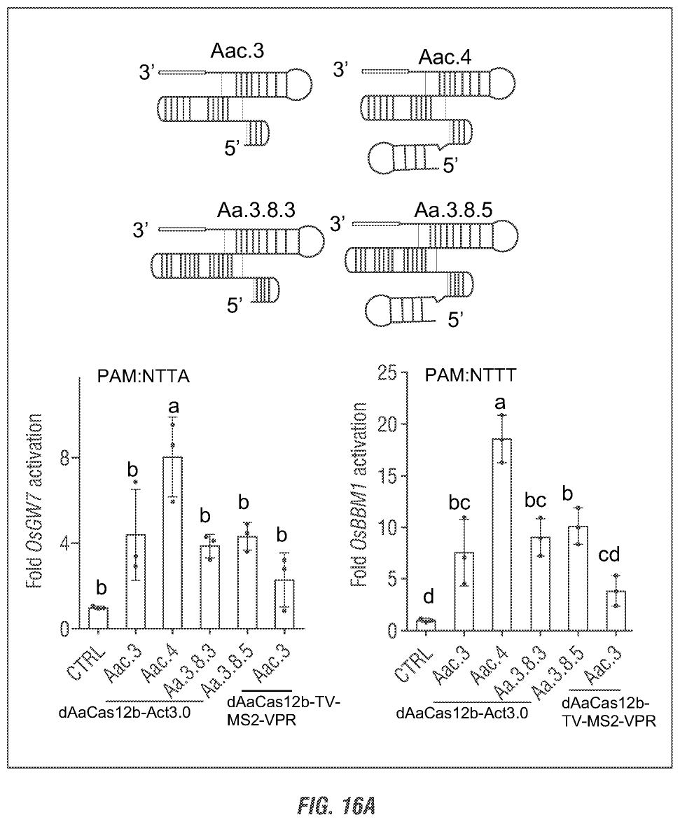

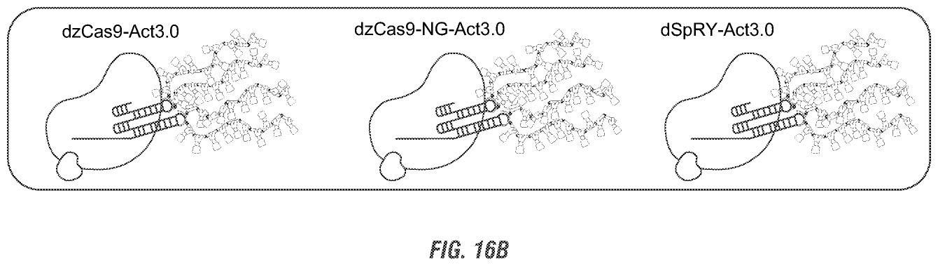

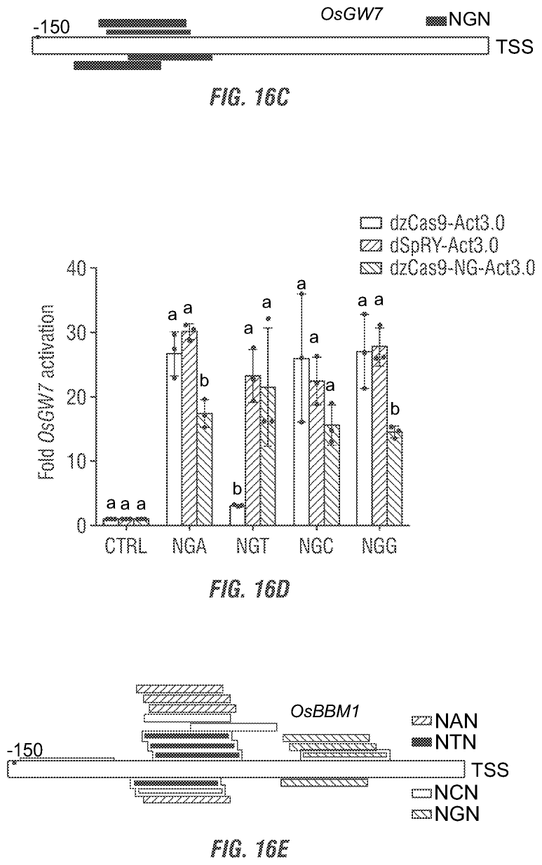

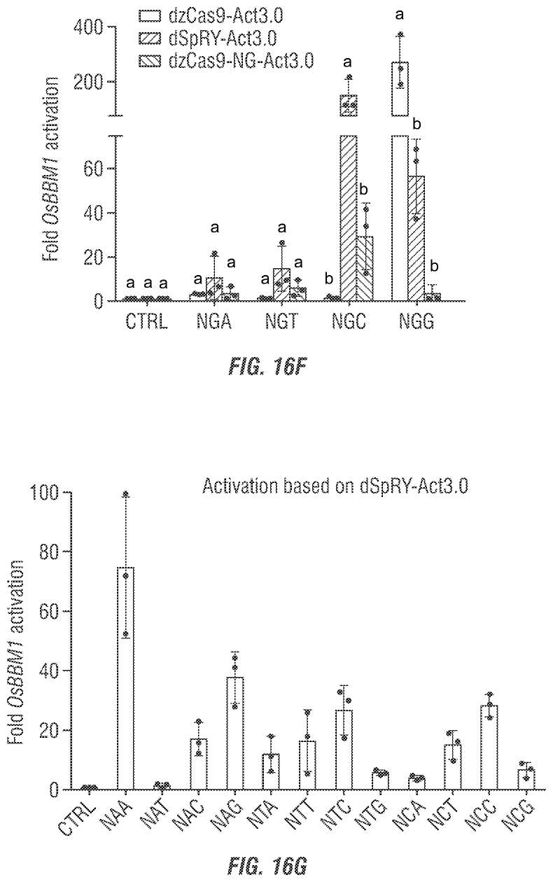

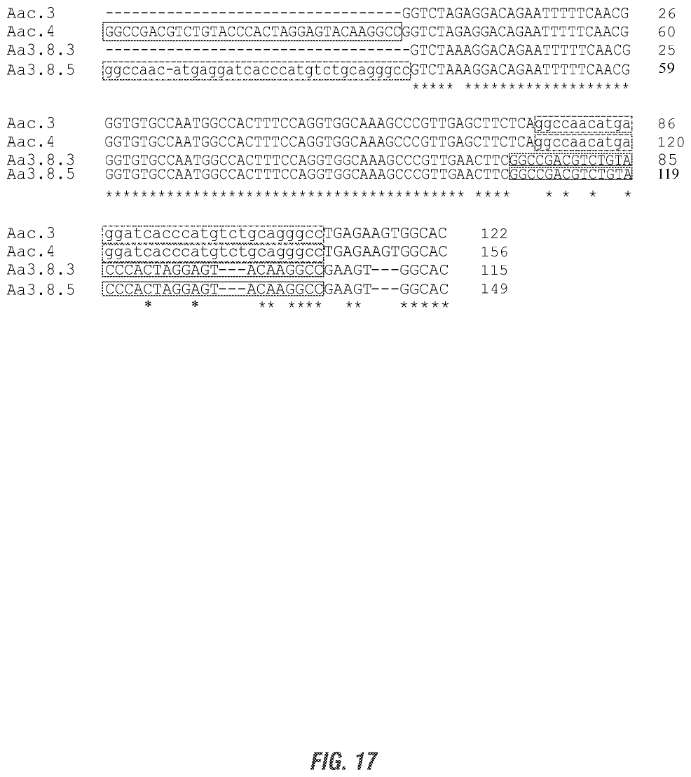

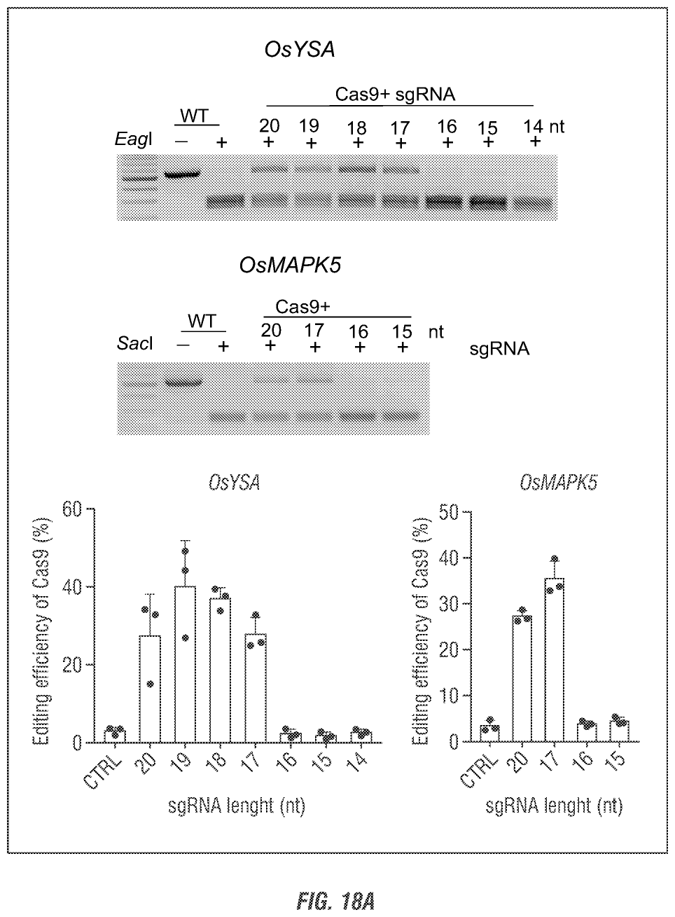

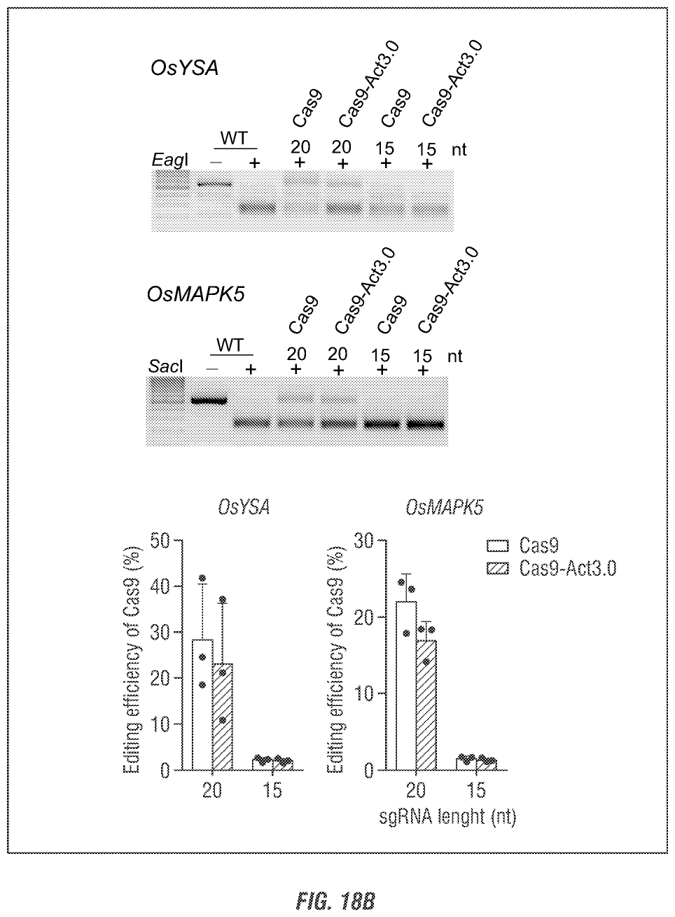

The narrow high-activity targeting window, high-activity GC contents and preference of targeting noncoding strand DNA would collectively limit the sgRNA choice in designing and implementing CRISPR-Act3.0 in plants. Additionally, it is also important to avoid targeting cis-regulatory elements so that binding of the CRISPR-Act3.0 components will not interfere with the recruitment of endogenous transcription factors and regulators necessary for transcription. In light of all these issues, it could be challenging to find many potentially good target sites for CRISPR-Act3.0 based on SpCas9, which recognizes NGG (N=A, C, G or T) protospacer adjacent motifs (PAMs). The limited target choices when targeting AtTCL1 in Arabidopsis may partly explain the relatively low level of gene activation that we observed for this gene (FIG. 14E). Many promoters in plants are A-T rich, making them difficult to target with SpCas9. Recently, we developed CRISPRa systems based on dCas12b proteins, which recognizes VTTV (V=A, C and G) PAMs. The most efficient Cas12b activation system used Aac.3 sgRNA scaffold that contains one MS2 stem loop (FIG. 16A). We were curious whether we could transfer the SpCas9 based CRISPR-Act3.0 strategy into Cas12b systems. To this end, we adopted and engineered three additional sgRNA scaffolds, including Aa.3.8.3, Aac.4 and Aa.3.8.5 (FIG. 16A and FIG. 17), which were meant to use one or two MS2 stem loops to recruit 10xGCN4 and 2xTAD through the MS2-MCP interaction. We targeted OsGW7 as well as a morphogenic gene OsBBM1 in rice protoplasts. For both genes, Aac.3, Aa.3.8.3 and Aa.3.8.5 sgRNA scaffolds resulted in two-fold higher activation than our previously established dAaCas12b-TV-MS2-VPR activation system (FIG. 16A). Notably, the Aac.4 sgRNA scaffold that contains two MS2 stem loops generated four- to five-fold higher activation than the dAaCas12b-TV-MS2-VPR system (FIG. 16A). Thus, we have engineered an improved CRISPRa system based on AaCas12b and the Aac.4 sgRNA scaffold.

We however realized that the improved Cas12b activation system was not as strong as the SpCas9 based CRISPR-Act3.0 system. Subsequently, we decided to relax the PAM requirements of SpCas9 in CRISPR-Act3.0. One promising SpCas9 variant is Cas9-NG that recognizes NG PAMs in human cells and in plants. Another promising SpCas9 variant is SpRY, which was recently claimed as near-PAM-less since it can edit NR (R=G and A) PAM sites with high efficiency and NY (Y=C and T) PAM sites with relatively low efficiency. To compare both SpCas9 variants, we engineered dzCas9-NG-Act3.0 and dSpRY-Act3.0 (based on the same maize codon-optimized Cas9) (FIG. 16B) and compared them with the dzCas9-Act3.0 at targeting nearly the same location at four NGN (NGA, NGT, NGC and NGG) PAM sites in the promoter of OsGW7 (FIG. 16C). Because SpCas9 can target NGA PAMs, dzCas9-Act3.0 generated robust activation (>20-fold) as expected at the canonical NGG PAM site and the non-canonical NGA PAM sites (FIG. 16D). Interestingly, we also observed high levels of activation with dzCas9-Act3.0 at the NGC PAM site, but not at the NGT PAM site (FIG. 16D). Impressively, dSpRY-Act3.0 resulted in >20-fold target gene activation at all four NGN PAM sites (FIG. 16D). By contrast, the dzCas9-NG-Act3.0 generated lower than 20-fold activation at three NGN PAM sites (FIG. 16D). We then compared these systems at a second gene, OsBBM1, at four overlapping targeting sites with four NGN (NGA, NGT, NGC and NGG) PAM sites closer to the TSS (FIG. 16E). At this gene, dzCas9-Act3.0 was only able to activate transcription through an NGG PAM-targeting sgRNA (FIG. 16F). By contrast, dSpRY-Act3.0 activated the targets at all four NGN PAM sites (fold-activation ranging from 10 to 200) and outperformed dzCas9-NG-Act3.0 at all these target sites (FIG. 16F). These results demonstrated that dSpRY-Act3.0 targets NGN PAMs more efficiently than dzCas9-NG-Act3.0. However, for targeting NGG PAM sites, the dzCas9-Act3.0 induced a higher efficiency than dSpRY-Act3.0. To determine whether dSpRY-Act3.0 could target NAN, NTN and NCN PAMs for gene activation, we picked 12 target sites with NNN PAMs (4 NAN, 4 NTN and 4 NCN) further from the TSS of OsBBM1 and these target sites were largely overlapped for close comparisons (FIG. 16E). Remarkably, the data showed that, with dSpRY-Act3.0, 11 out of 12 sgRNAs resulted in activation of OsBBM1 (FIG. 16G). Together, dSpRY-Act3.0 appears to enable near-PAM-less gene activation in plants, consistent with its near-PAM-less gene editing in human cells.

### Discussion for Examples 1-5

In plant functional genomics, a central question is to define the causal relationships between gene expression and phenotypic features in plants. The CRISPRa represents a promising approach to streamline and expedite such research by targeting gene activation in plants. To improve activation potency, targeting flexibility and scalability of CRISPRa in plants, we applied an engineering approach to systemically exploit different sgRNA scaffolds and transcription activators to develop the next-generation CRISPRa systems. We successfully developed CRISPR-Act3.0, which consists of dCas9-VP64, gR2.0 scaffold with 2xMS2 stem loops, 10xGCN4 SunTag fused to RNA binding protein MCP and 2xTAD activators fused to scFv (FIG. 3A). We benchmarked CRISPR-Act3.0 as a 3^{rd} generation CRISPRa system in plants as it significantly outperformed all the 2^{nd} generation CRISPRa systems in rice assays (FIG. 3D and FIG. 4B). In CRISPR-Act3.0, multiple 2xTAD activators were recruited by the sgRNA scaffold through the MS2-MCP interaction. This feature may allow us to further develop complex CRISPRa systems with additional functionality through engineering orthogonal sgRNA scaffolds (see Examples 6-9).

To make the CRISPR-Act3.0 systems user-friendly, we developed an efficient toolbox for multiplexed sgRNAs assembly of up to six gRNA2.0 cassettes in one step based on polymerase chain reaction (PCR)-free modular Golden Gate cloning and Gateway cloning systems (FIG. 5A and FIG. 6). We demonstrated that the CRISPR-Act3.0 coupled with the M-tRNA systems enabled multiplexed gene activation with simultaneous activation of several enzyme-encoding genes involved in the β-carotene pathway as well as in the proanthocyanidin pathway in rice (FIG. 5C-E and FIG. 8-10), suggesting the promising application of CRISPR-Act3.0 in plant metabolic engineering. Notably, we also demonstrated CRISPR-Act3.0 for simultaneous modification of two independent traits (flowering and trichome development) through multiplexed gene activation in Arabidopsis (FIG. 14). Strikingly, the Arabidopsis plants with the AtFT activation nearly reduced the plant life cycle by half, e.g., ˜30 days (FIG. 14D). The phenotypes of early flowering and reduced trichome numbers due to simultaneous gene activation were stably transmitted to the T3 generation (FIG. 14E-F). These results suggest that the CRISPR-Act3.0 holds great promise to accelerate crop breeding. Furthermore, we showed potent activation of a morphogenic gene OsBBM1 in rice, with up to 300-fold activation (FIG. 4B and FIG. 16F). Our demonstration will promote future use of CRISPR-Act3.0 for activating endogenous morphogenic genes to promote genotype-independent plant regeneration, as opposed to using heterologous morphogenic gene expression systems. While both dpcoCas9-Act3.0 and dzCas9-Act3.0 showed comparable activation potency, we observed that the dzCas9-Act3.0 system is more robust as it does not cause plasmid DNA recombination when combined with M-tRNA or M-U3 sgRNAs and expressed in A. tumefaciens strains (FIG. 12).

Previous studies have demonstrated that the CRISPRa potency is highly sensitive to sgRNA target position relative to the TSS. The optimal targeting window for CRISPRa in mammalian cells, bacteria and plants had been reported to be the 200 bp, 60-90 bp and 350 bp upstream region of the TSS. However, only limited sgRNAs and a few genes were tested in these studies. By analyzing activation data from 16 genes with 56 sgRNAs, we identified the −0 bp to −200 bp region from TSS as a high activity window for CRISPR-Act3.0 based gene activation in plants (FIG. 15A), which is similar to that of the dCas9-TV system. Interestingly, we found that sgRNAs targeting the noncoding strand DNA and with balanced GC contents are more likely to result in high levels of gene activation (FIG. 15A-B). However, to ensure efficient activation, a prescreen step for best sgRNAs using a cell-based assay is recommended. These observations also suggest it is important to broaden the targeting scope for successful implementation of CRISPRa in plants.

Toward this end, we developed an improved dAaCas12b-based activation system for targeting VTTV PAMs with a new engineered sgRNA scaffold Aac.4 (FIG. 16A), although the activation potency was still not as strong as dCas9 based CRISPR-Act3.0. To further broaden the targeting scope of dCas9 based CRISPR-Act3.0, we developed dzCas9-NG-Act3.0 based on Cas9-NG and dSpRY-Act3.0 based on SpRY. Notably, our data suggest dSpRY-Act3.0 can achieve near-PAM-less gene activation, which worked well particularly at NGN and NAN PAMs (FIG. 16D-G). Consistently, three recent studies demonstrated that SpRY could be used in genome editing at all NNN PAMs and exhibited a preference of NGN and NAN PAMs in rice. Notably, high-frequency SpRY-mediated T-DNA self-editing was observed. The T-DNA self-targeting property could have compromised the activation potency of dSpRY-Act3.0 in our rice protoplast assays, due to high copy numbers of plasmids used. Consequently, dSpRY-Act3.0 may induce higher activation efficiency in stable plants, which however needs to be tested. Taken together, these results demonstrate the high flexibility and adaptability of the CRISPR-Act3.0 strategy, which can be efficiently adopted to other CRISPR-Cas systems. The development of dAaCas12b-Act3.0 and dSpRY-Act3.0 systems greatly reduces the PAM restriction and provides users with high flexibility in choosing targeting sites.

In conclusion, we have developed a highly efficient CRISPR-Act3.0 toolbox for multiplexed gene activation in plants, which would aid many applications including rewiring metabolic pathways, investigating gene regulatory networks, and genome-wide screens for identifying key genes in regulating plant development and stress responses.

### Example 6: Development of the CRISPR-Combo System for Simultaneous Genome Editing and Gene Activation in Plants

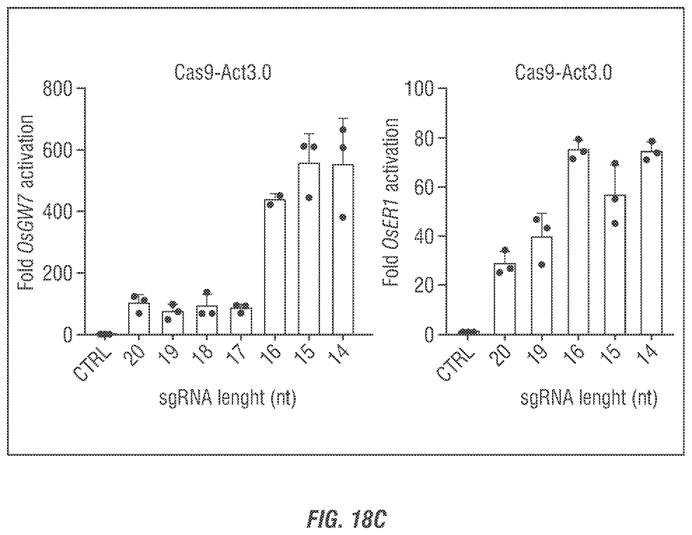

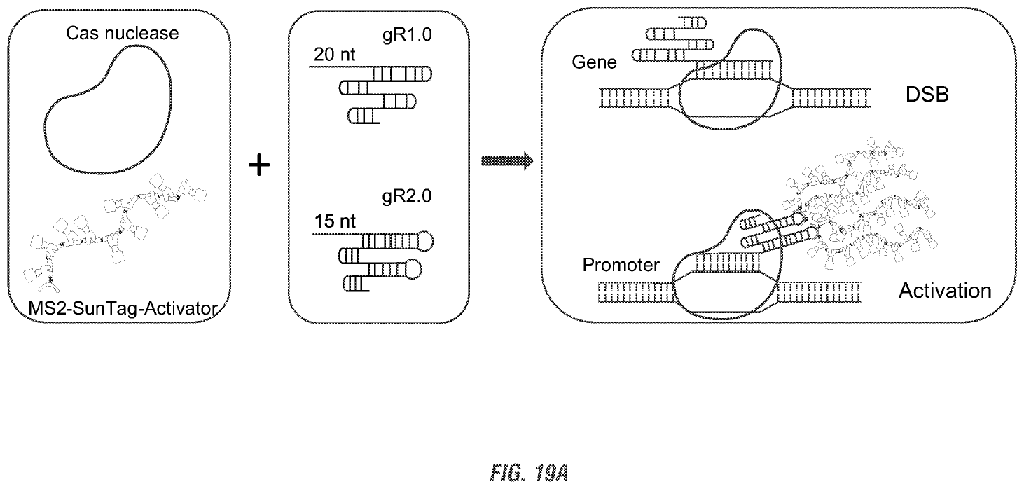

To assess inactivation of CRISPR-Cas9′s DNA cleavage activity through sgRNA engineering, we tested targeted mutagenesis by Cas9 at OsYSA and OsMAPK5 loci using differ protospacer lengths in rice protoplasts. Restriction fragment length polymorphism (RFLP) analysis showed that 17 to 20-nt protospacers conferred efficient mutagenesis while 14 to 16-nt protospacers were unable to cause mutations at the target site (FIG. 18A). We created the CRISPR-Cas9-Act3.0 (Cas9-Act3.0) that was based on the wildtype Cas9. Close comparison Cas9-Act3.0 and Cas9 showed that they had comparable editing activities at two independent targets sites when coupled with sgRNAs of 20-nt protospacers (FIG. 18B). Reduction of the protospacers to 15-nt abolished editing activity of both systems (FIG. 18B). These data suggest Cas9-Act3.0 possesses the wildtype level of Cas9 nuclease activity, which can be turned off with short protospacers. Next, we assessed gene activation by Cas9-Act3.0 with the sgRNA2.0 scaffold of variable protospacer lengths. Interestingly, robust transcriptional activation of the target genes (OsGW7 and OsER1) was observed with short 14-16-nt protospacers (FIG. 18C). By contrast, longer protospacers (e.g., 17 to 20-nt) showed reduced gene activation efficiency (FIG. 18C), suggesting targeted mutagenesis had occurred at these sites which prevented the binding of the activation system to the promoters once mutated.

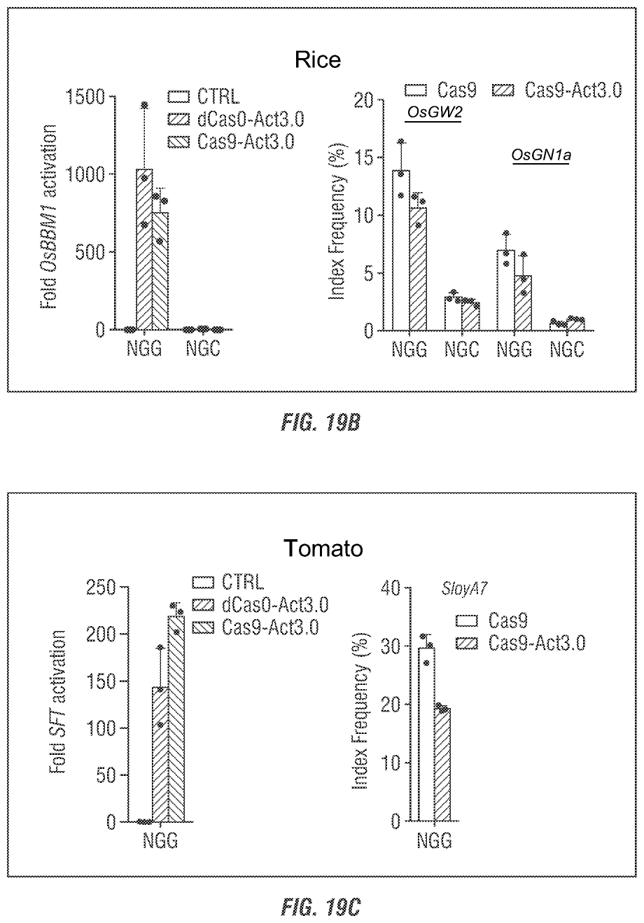

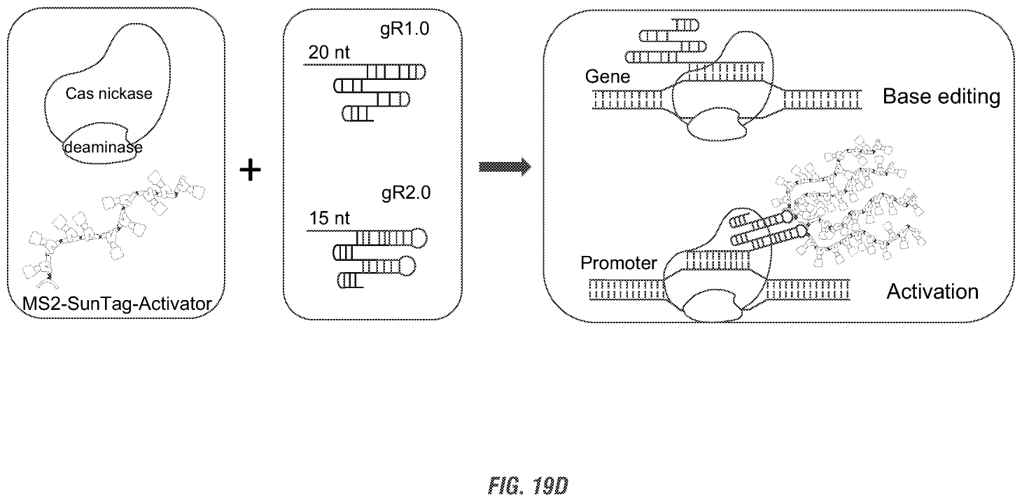

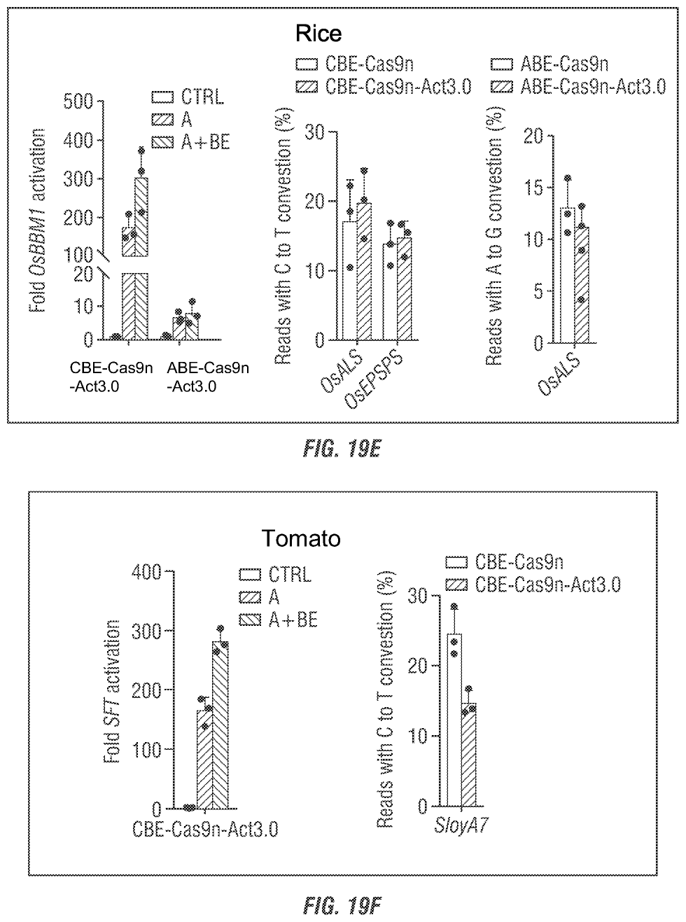

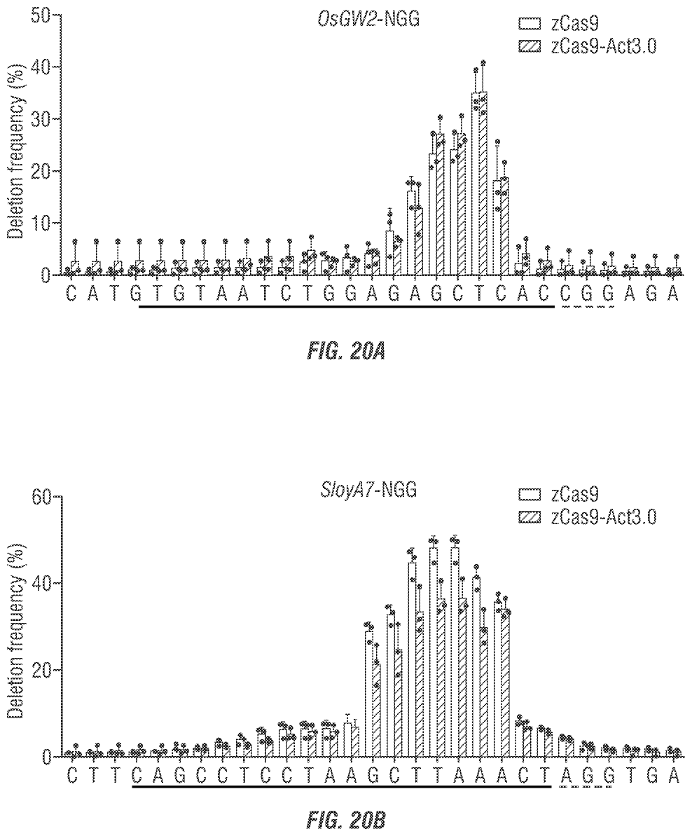

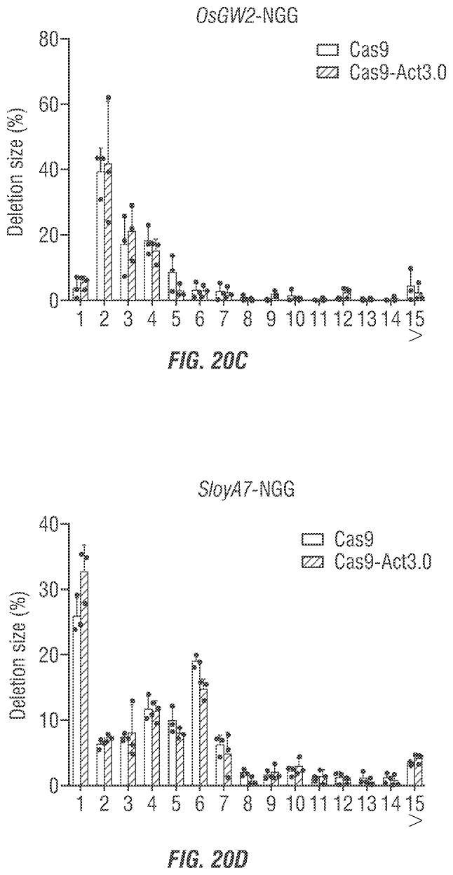

The above experiments proved the principle of a first CRISPR-Combo system that allows for simultaneous genome editing and gene activation in an orthogonal manner programmed by the normal sgRNA (gR1.0) and sgRNA-2.0 (gR2.0) with 20-nt protospacers and 15-nt protospacers, respectively (FIG. 19A). To benchmark this CRISPR-Combo system, we further tested the principle whether Cas9-Act3.0 enables orthogonal gene activation and knockout by simultaneous targeting OsBBM1, OsGW2 and OsGN1a at both NGG and NGC protospacer adjacent motifs (PAMs) (FIG. 19B). At the canonical NGG protospacer adjacent motif (PAM) site, Cas9-Act3.0 in the CRISPR-Combo system showed comparable gene activation level to that of dCas9-Act3.0 (FIG. 19B). Both systems failed to activate the target gene at an NGC non-canonical PAM site, likely due to PAM incompatibility (FIG. 19B). We next compared Cas9-Act3.0 with Cas9 at editing two NGG PAM sites and two NGC PAM sites. Both systems showed higher editing efficiency at the preferred NGG PAM sites (FIG. 19B). Importantly, there was no difference between Cas9-Act3.0 and Cas9 on genome editing activity (FIG. 19B). We further assessed the system in tomato by simultaneously targeted activation of SFT and targeted mutagenesis of SloyA7, and similar results were observed in tomato protoplasts (FIG. 19C). Furthermore, Cas9-Act3.0 resulted in similar NHEJ mutation profiles to those of Cas9 at the target sites in rice and tomato, either for deletion positions (FIG. 20A-B) or deletion sizes (FIG. 20C-D).

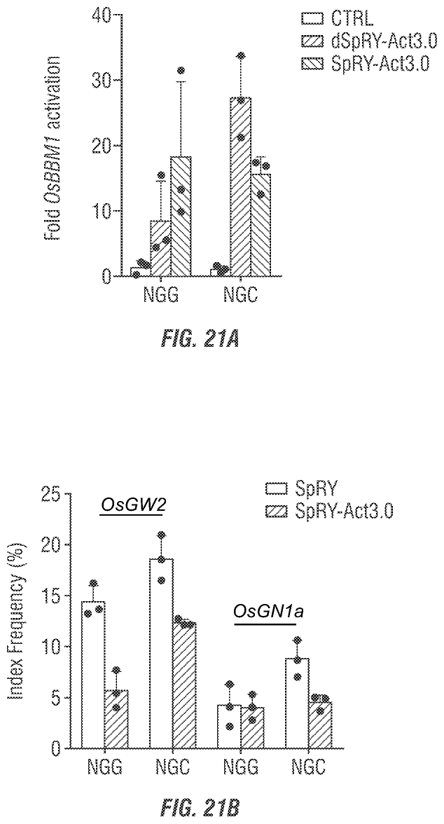

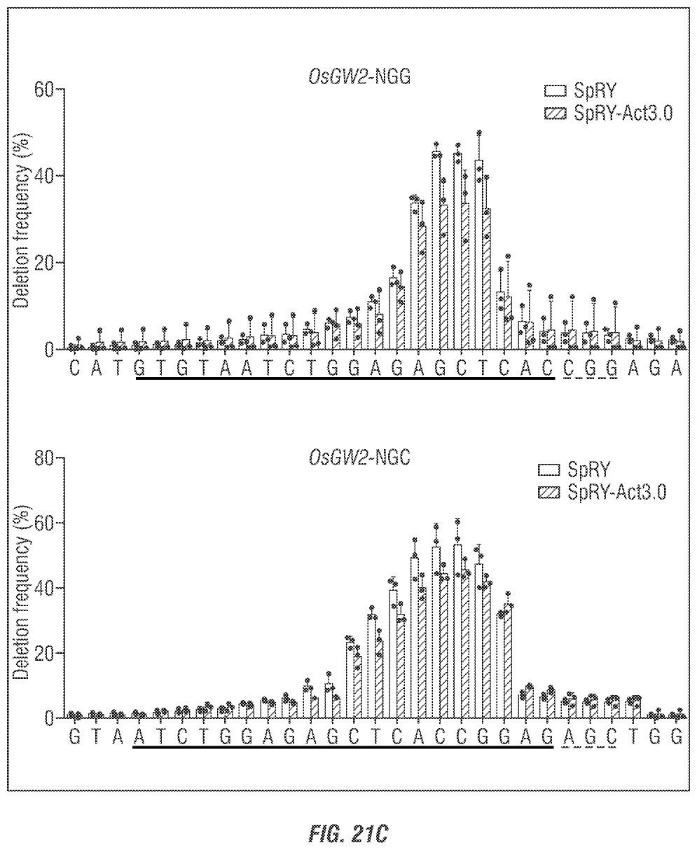

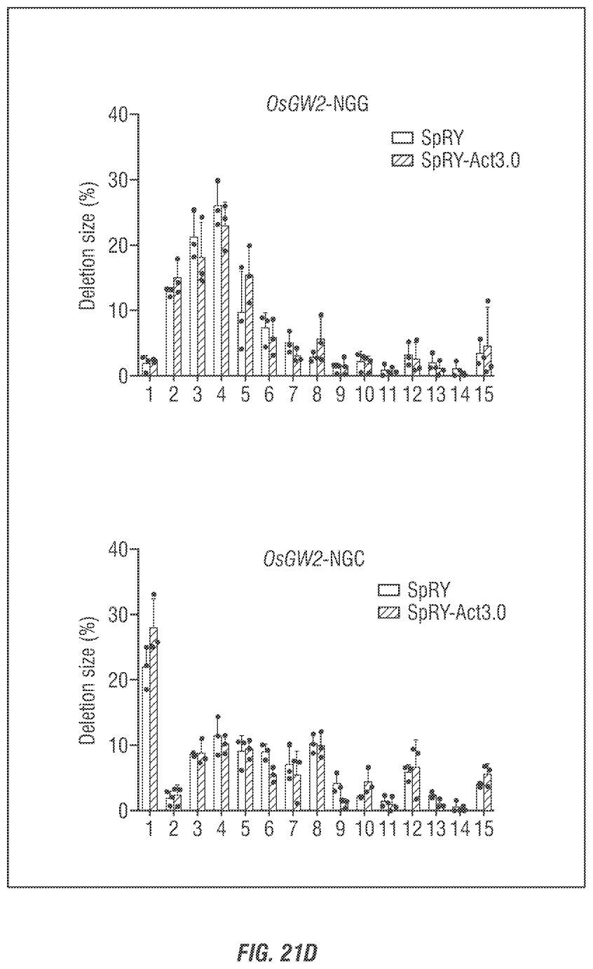

Recently, Cas9 variant SpRY was demonstrated for PAM-less genome editing in human cells and plants. Based on the CRISPR-Combo principle, we further demonstrated that SpRY-Act3.0 enables orthogonal gene activation and knockout by simultaneous targeting OsBBM1, OsGW2 and OsGN1a at both NGG and NGC PAMs (FIG. 21A). At the both NGG and NGC PAM sites, SpRY-Act3.0 systems induced comparable activation efficiencies about ˜10 to 30-fold gene activation compared to the dSpRY-Act3.0 system (FIG. 21A). We further compared SpRY-Act3.0 and SpRY for genome editing at the same four target sites at OsGW2 and OsGN1a. The data showed that SpRY-Act3.0 retained genome editing capability, albeit with slightly lower editing efficiency at three out of four target sites (FIG. 21B). Furthermore, SpRY-Act3.0 and SpRY showed similar NHEJ mutation profiles (FIG. 21C-D). These results suggest SpRY-Act3.0 allows for simultaneous genome editing and gene activation at relaxed PAM sites.

### Example 7: Development of the CRISPR-Combo System for Simultaneous Base Editing and Gene Activation in Plants

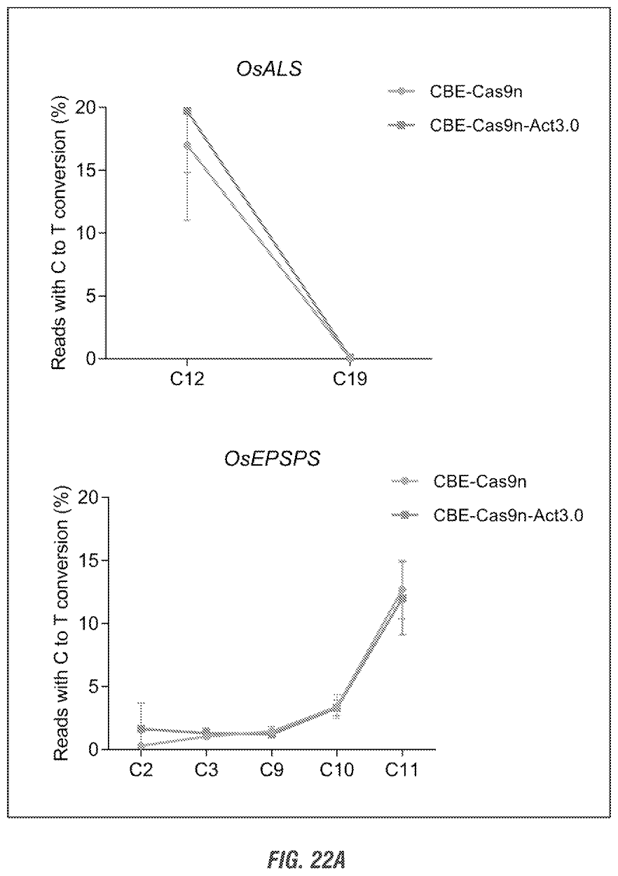

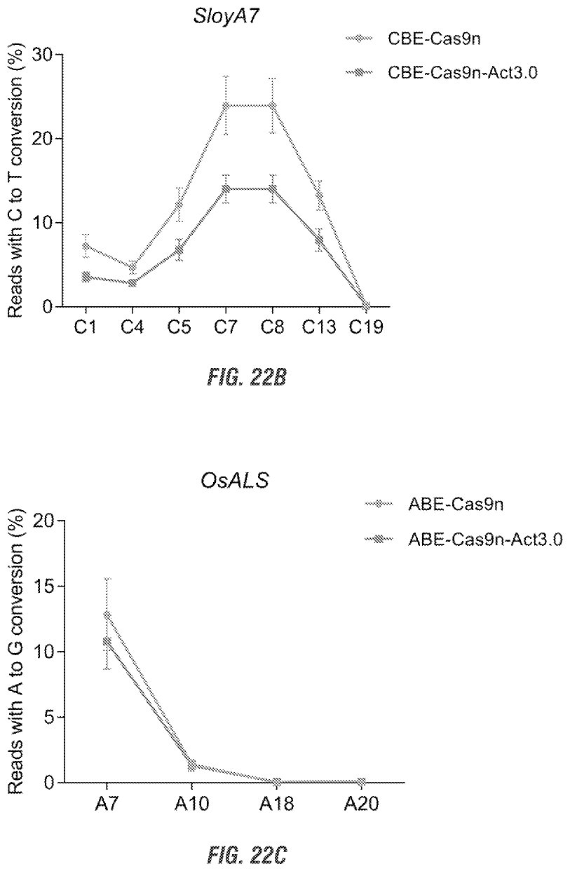

We next sought to develop CRISPR-Combo systems suitable for simultaneous base editing and gene activation. For C-to-T base editing, CBE-Cas9n-Act3.0 was generated by implanting the highly efficient A3A/Y130E-Cas9-UGI into the CRISPR-Act3.0 system (FIG. 19D). For A-to-G base editing, ABE8e-Cas9n was used to generate ABE-Cas9n-Act3.0 (FIG. 19D). These BE-Cas9n-Act3.0 systems were first assessed in rice protoplasts by simultaneous targeting OsBBM1, OsALS and OsEPSPS. We found that the CRISPR-Combo vectors capable of doing simultaneous base editing and gene activation resulted in similar gene activation levels to those that contained activation sgRNAs alone, although adenosine deaminase ABE8e drastically reduced the activation potency of the corresponding ABE-Cas9n-ABE system (FIG. 19E). However, the CBE-Cas9n-Act3.0 and ABE-Cas9n-Act3.0 systems had similar base editing activity to the CBE-Cas9n and ABE-Cas9n systems (FIG. 19E). We further test the CBE-Cas9n-Act3.0 in tomato protoplasts. The results showed high gene activation potency when both activation and base editing sgRNAs were co-expressed (FIG. 19F) and there was a slight reduction of base editing efficiency by CBE-Cas9n-Act3.0 when compared to the canonical base editor, CBE-Cas9n (FIG. 19F). However, compared to the canonical base editors, the base editing windows were not altered for either CBE-Cas9n-Act3.0 or ABE-Cas9n-Act3.0 (FIG. 22).

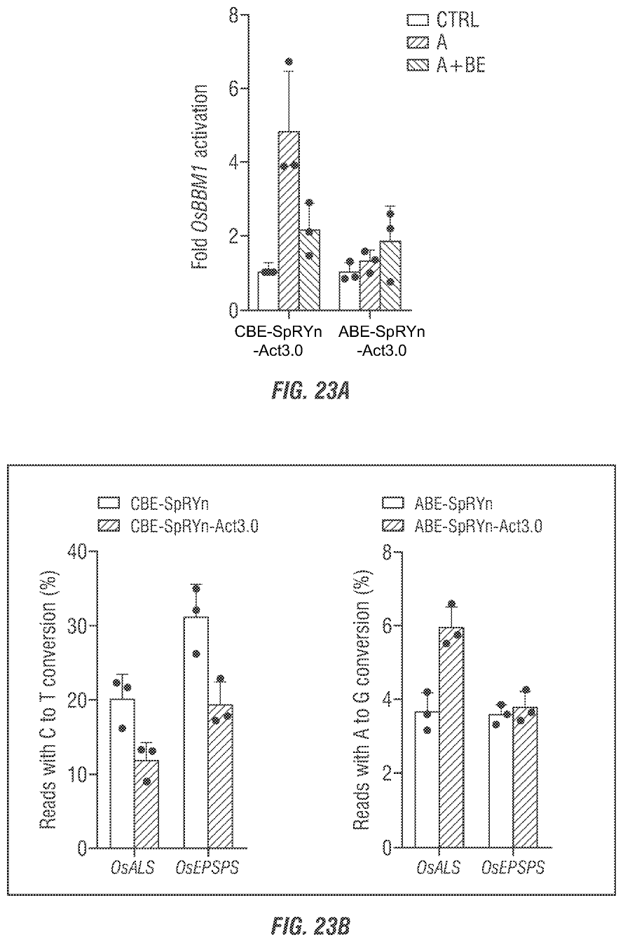

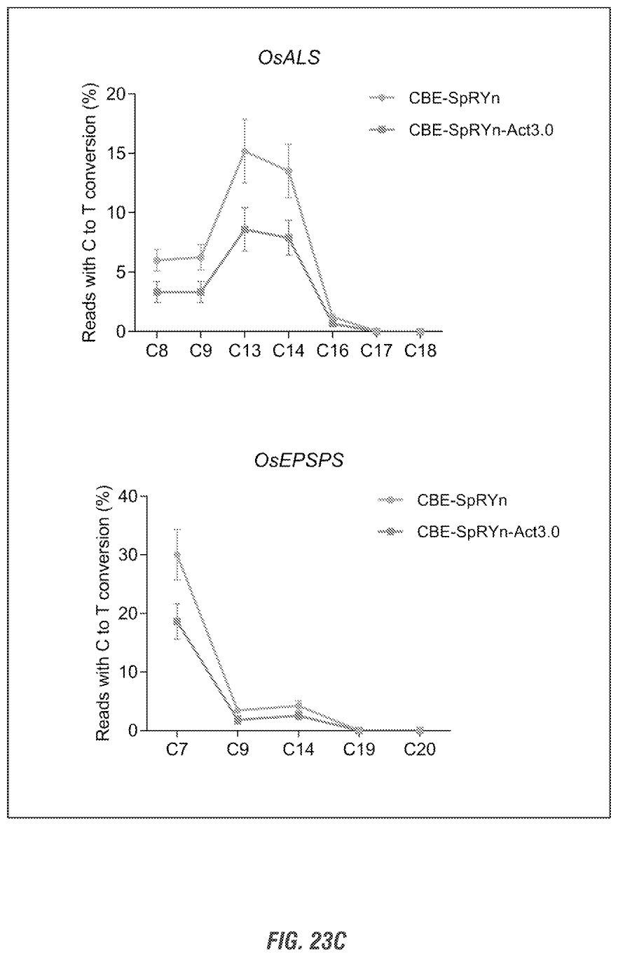

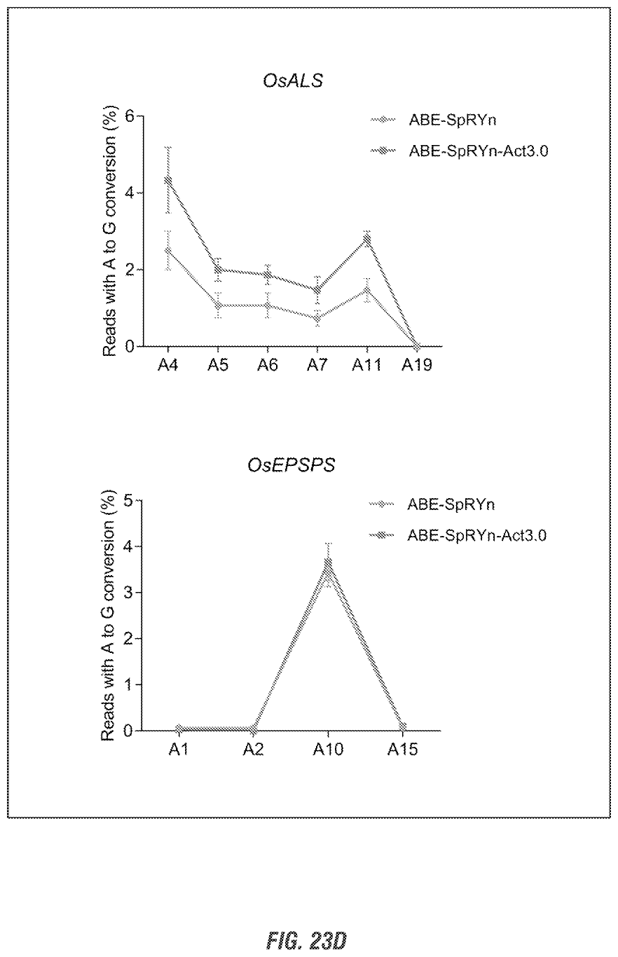

To broaden the targeting scope, we also generated CBE-SpRYn-Act3.0 and ABE-SpRYn-Act3.0. By simultaneously targeting OsBBM1, OsALS and OsEPSPS, we found CBE-SpRYn-Act3.0 only generated a low level of gene activation of OsBBM1 in rice protoplasts, while ABE-SpRYn-Act3.0 failed for gene activation (FIG. 23A), consistent with previous observation of low activation potency of dSpRY-Act3.0. However, both CBE-SpRYn-Act3.0 and ABE-SpRYn-Act3.0 systems yielded relatively comparable base editing efficiency (FIG. 23B) and base editing windows to the canonical CBE and ABE controls (FIG. 23C).

### Example 8: Accelerated Breeding of Transgene-Free Genome-Edited Plants with CRISPR-Combo Based Florigen Activation

The CRISPR-Combo systems are enabling technologies due to their ability for simultaneous gene editing and activation. For demonstration in this study, we decided to focus on addressing some of the most pressing challenges in plant genome editing. The first change is on achieving accelerated breeding of genome-edited transgene-free plants. We recently showed activation of the florigen gene FT in Arabidopsis by CRISPR-Act3.0 promoted early flowering. We reasoned that a genome editing pipeline with simultaneous activation of such a florigen gene using CRISPR-Combo would have three benefits compared to the traditional genome editing experiments. First, it would drastically reduce the plant breeding life cycle. Second, the transgenic plants with extra-early flowering phenotypes (plants showed four leaves when flower buds became visible) would suggest high levels of CRISPR-Combo expression, indicating high levels of genome editing in these lines. Hence, the easy-to-score extra-early flowering phenotype would indicate high-efficiency genome editing, saving much effort for molecular genotyping. Third, selection of normal flowering plants in the next generation of genome-edited early flowering plants would drastically reduce the effort of genotyping for transgene-free genome-edited plants by at least 75%, based on the Mendelian segregation pattern of a single transgene.

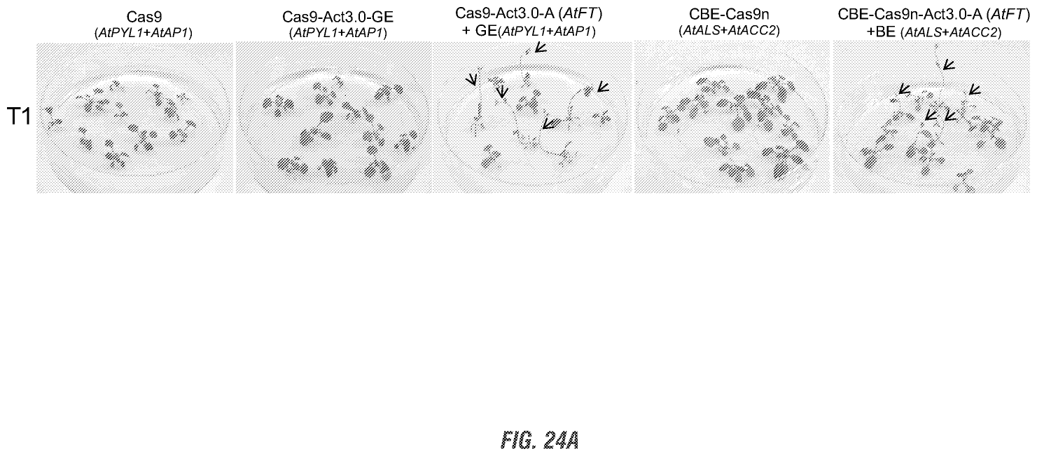

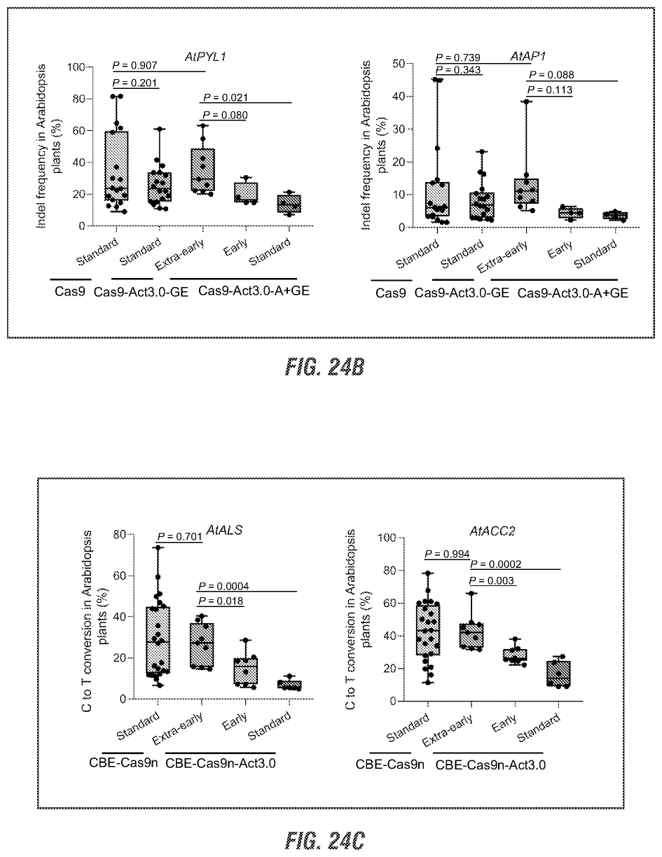

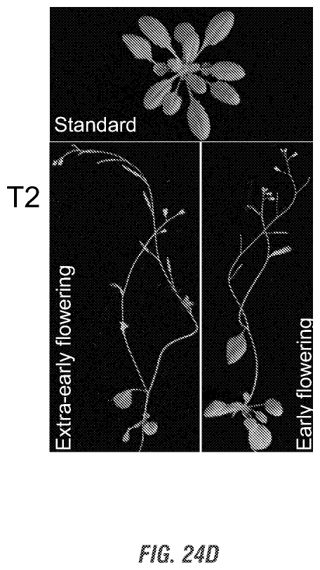

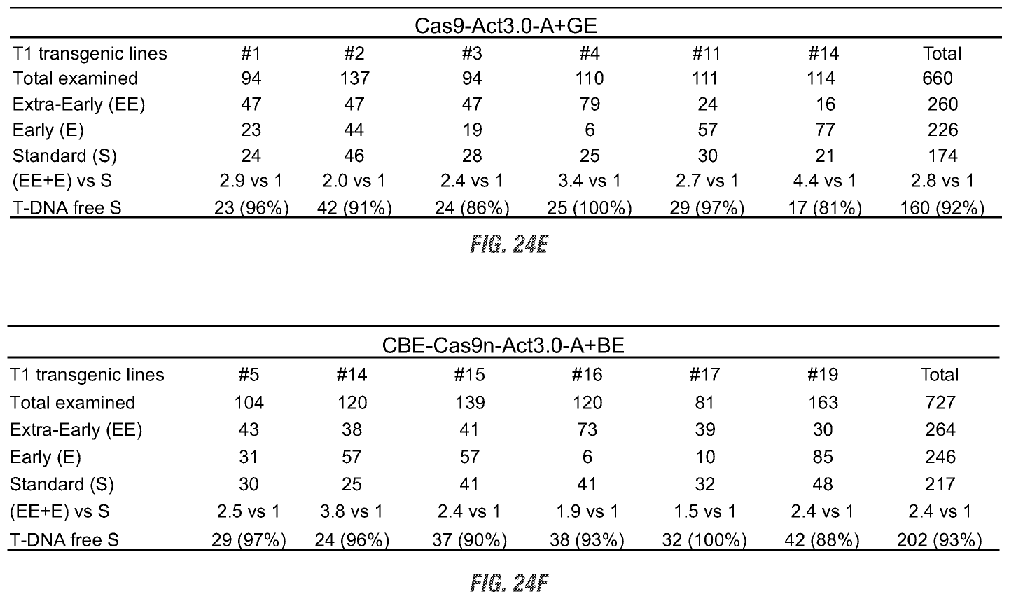

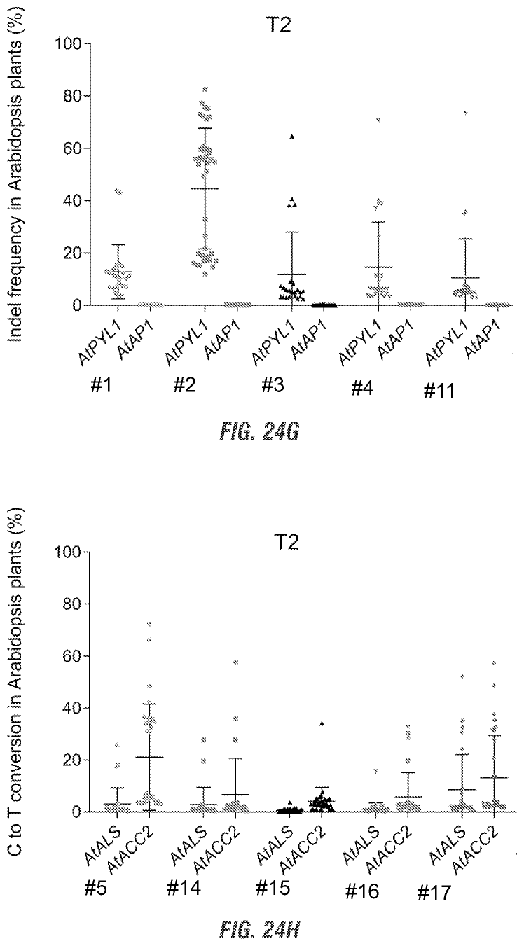

To assess this improved genome editing pipeline, we decided to test two CRISPR-Combo systems, Cas9-Act3.0 and CBE-Cas9n-Act3.0, in Arabidopsis. For Cas9-Act3.0, multiplexed gene editing (GE) of AtPYL1 and AtAP1 was pursued with simultaneous activation of AtFT, with three different sgRNAs according to the design guideline (FIG. 19A). Extra-early flowering T1 plants were readily observed for this construct, but not for Cas9 genome editing control construct or the Cas9-Act3.0 control without AtFT activation (FIG. 24A). Similarly, a multiplexed CBE-Cas9n-Act3.0 construct was made for simultaneous base editing of two herbicide target genes (AtALS and AtACC2) with concurrent activation of AtFT This construct, not the CBE-nCas9 base editor control, also resulted in T1 transformants with extra-early flowering phenotypes (FIG. 19B). We quantified gene editing frequencies for all T1 lines using next-generation sequencing (NGS) of PCR amplicons. For Cas9 and Cas9-Act3.0-GE (without AtFT activation) constructs, both systems produced comparable editing frequencies at the AtPYL1 and AtAP1 sites (FIG. 24B). For the Cas9-Act3.0-A+GE construct for simultaneous gene activation and editing, T1 plants were classified into three groups: extra-early flowering, early flowering, and standard (e.g., wild type-like flowering time). Extra-early flowering plants showed overall higher genome editing frequencies, especially compared to the standard flowering plants (P=0.021 at AtPYL1 and P=0.088 at AtAP1) (FIG. 24B). Compared to the standard plants resulted from the Cas9 and Cas9-Act3.0-GE constructs, the median editing frequencies were elevated for Cas9-Act3.0-A+GE extra-early flowering plants (FIG. 24B). We next quantified C-to-T base editing frequencies for the T1 plants from the multiplexed CBE-Cas9n-Act3.0 construct. The extra-early flowering plants showed significantly higher base editing frequencies than the early flowering (P=0.018 and P=0.003) and standard plants (P=0.0004 and P=0.0002) at the AtALS site and the AtACC2 site, respectively (FIG. 24C). Compared to the conventional CBE-Cas9n vector, the dynamic ranges of base editing frequencies were much narrower for the extra-early flowering plants of the CBE-Cas9n-Act3.0-A+BE construct at both target sites (FIG. 24C), suggesting extra-early flowering plants had more robust editing across the population.

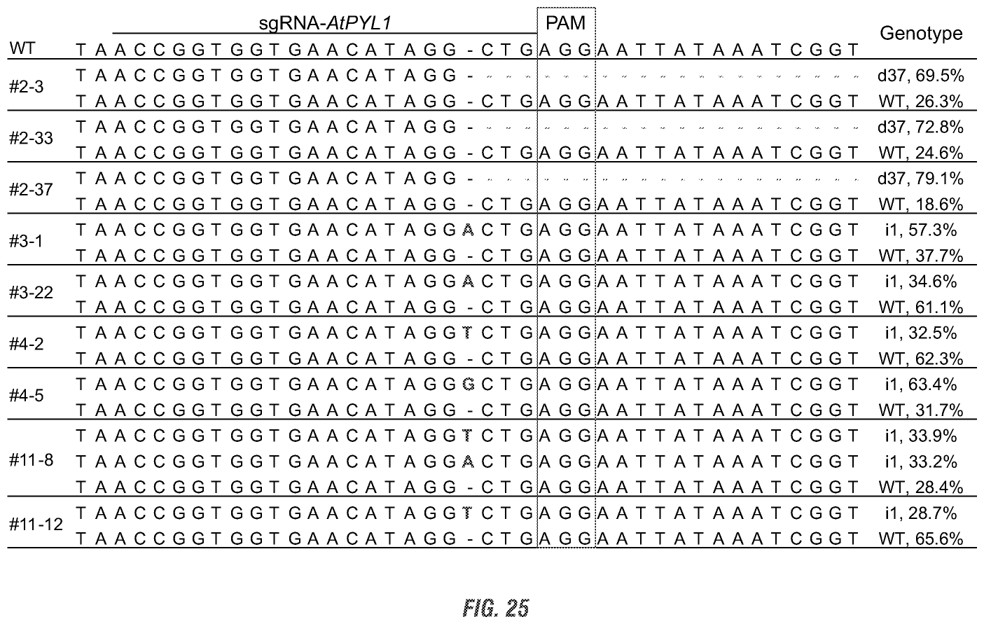

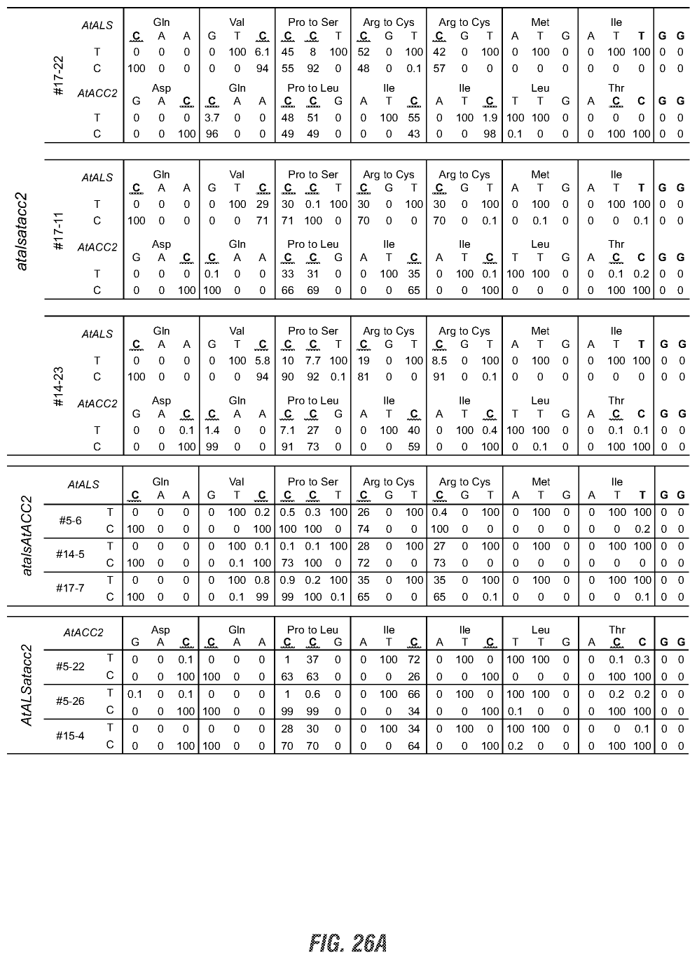

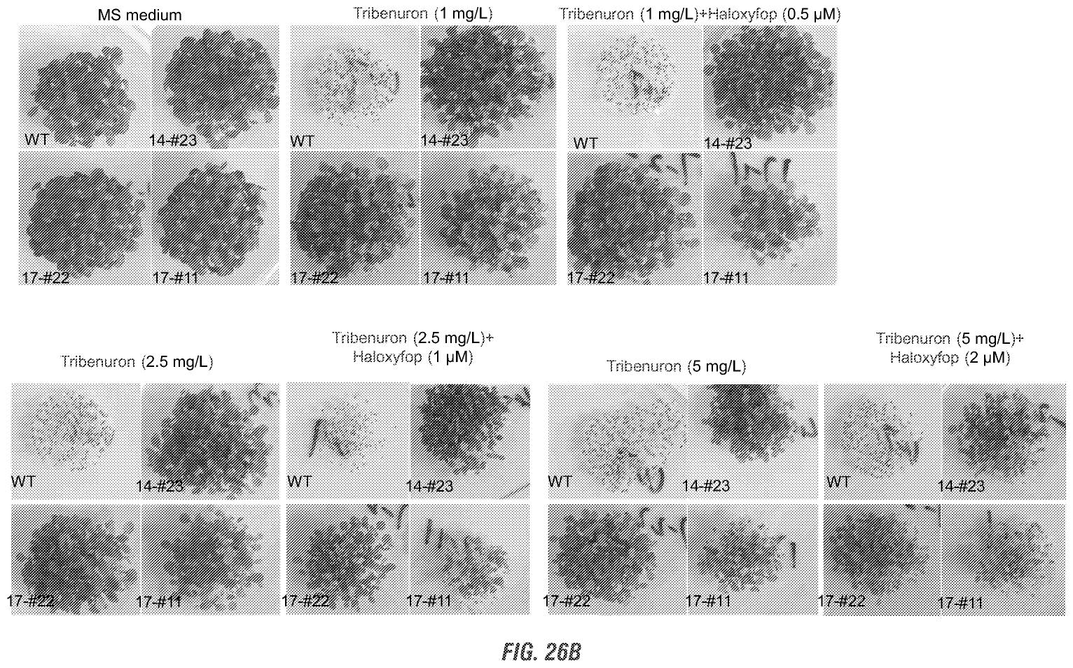

We next sought to evaluate whether the early flowering phenotype could be reliably used as a phenotypic marker for transgenic plants in the next (T2) generation. We focused on the progeny of some extra-early flowering T1 plants. Plants from each T2 population were again classified as extra-early flowering, early flowering, and standard (FIG. 24D). For the Cas9-Act3.0-A+GE construct, six T2 populations were examined, with numbers of plants ranging from 94 to 137 per population. The ratios of all early flowering plants to the standard plants averaged 2.8 to 1 (FIG. 24E), indicating nearly all the parental T1 lines carried only one T-DNA integration event. Remarkably, PCR-based genotyping confirmed that standard plants are indeed mostly T-DAN free plants with an average of 92% accuracy (FIG. 24E). Similar results were found for the T2 plants from the CBE-Cas9n-Act3.0-A+BE T1 lines (FIG. 24F), where standard plants were confirmed by molecular genotyping as T-DNA free plants with an average of 93% accuracy (FIG. 24F). To identified genome edited lines in these transgene-free plants, we genotyped these T2 lines by NGS. For the Cas9-Act3.0-A+GE construct, all five T2 populations generated detectable mutants with line #2 having the highest number of genome-edited lines with a median editing frequency of ˜50% at the AtPYL1 site (FIG. 24G). Transgene-free monoallelic mutants were readily identified in these populations (FIG. 25). It is of note that the AtAP1 site was barely mutated, which is probably due to the low editing activity of the sgRNA, resulting in very low frequency of germline transmittable mutations. For the CBE-Cas9n-Act3.0-A+BE construct, all five populations generated base-edited lines (FIG. 24G). The editing frequency at the AtACC2 site was higher than that of the AtALS site in each T2 population (FIG. 24G), which is consistent with the data in T1 lines (FIG. 24D). Nevertheless, transgene-free double mutants (atals ataac2) and single mutants (atals or ataac2) were readily identified in the standard T2 lines (FIG. 26A).

Three representative CBE-Cas9n-Act3.0-mediated atals atacc2 T2 lines 14-#23, 17-#11, and 17-#22 were selected to determine herbicide resistance. T2 line 14-#23 contains the P197F mutation of AtALS and the P1864L mutation of AtACC2. Both 17-#11, and 17-#22 contain the P197S mutation of AtALS and the P1864L mutation of AtACC2. We found that the descendants of T2 lines 14-#23, 17-#11, and 17-#22 were tribenuron-resistant, consistent with the previous report that P197F and P197S mutations of AtALS confer Arabidopsis plants with herbicide resistance (Chen at al., Sci China Life Sci (2017) 60:520-523). However, whether P1864L of AtACC2 would confer herbicide resistance was unknown. We found that the three atals atacc2 T2 lines' descendants could survive in the MS medium supplemented with both tribenuron and haloxyfop, indicating P1864L of AtACC2 confers Arabidopsis plants with haloxyfop resistance (FIG. 26B). These results highlight that the CRISPR-Combo base-editing system allows for fast-track creation (through activation of AtFT) of transgene-free edited plants with two independent herbicide resistance traits (through base editing of AtALS and At ACC2).

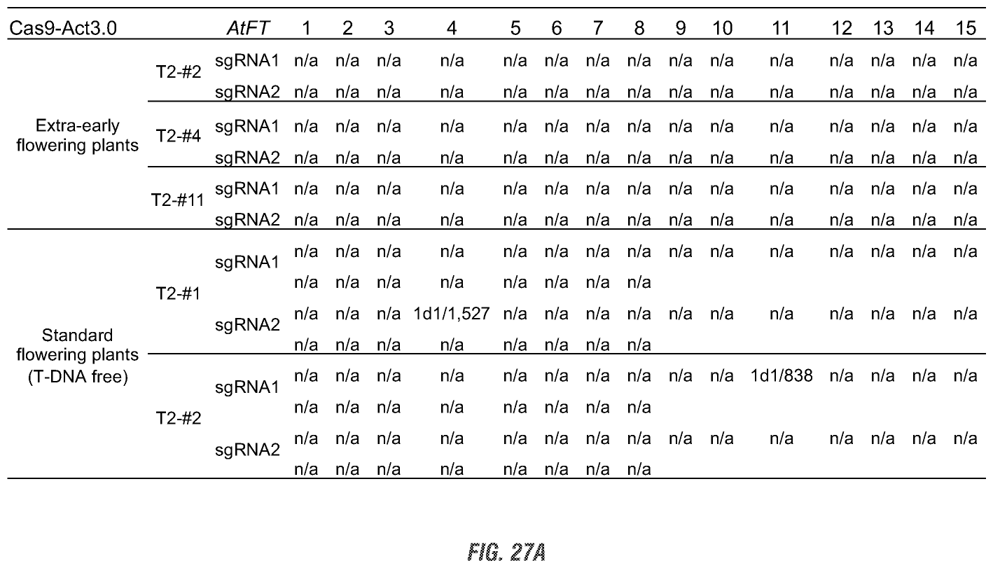

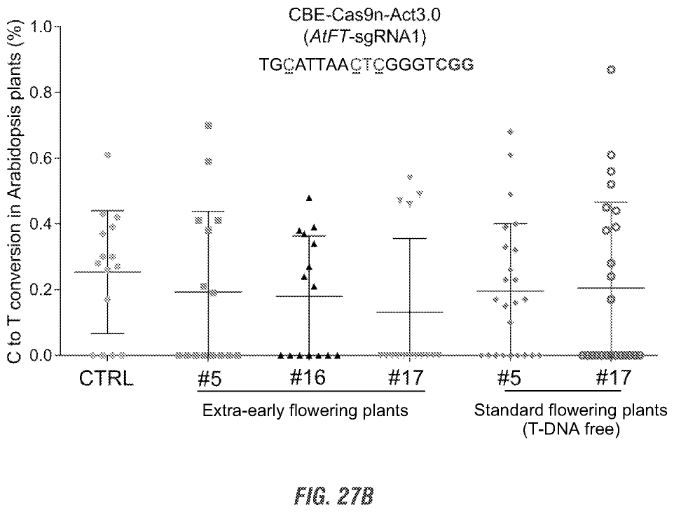

In addition, we investigated whether the Cas9-Act3.0-A+GE and CBE-Cas9n-Act3.0-A+BE constructs induced potential off-target events at AtFT target sites with a 15 nt sgRNA (FIG. 27A-B). In base-edited T2 extra-early flowering plants and transgene-free plants, no off-target event was detected at the AtFT sites for either Cas9-Act3.0 or CBE-Cas9n-Act3.0 systems. Altogether, these data demonstrated a CRISPR-Combo based pipeline for rapid generation of transgene-free genome-edited plants, whether for targeted indel mutations or for C-to-T base editing.

### Example 9: Accelerated Regeneration of Genome-Edited Plants with CRISPR-Combo Based Morphogenic Gene Activation

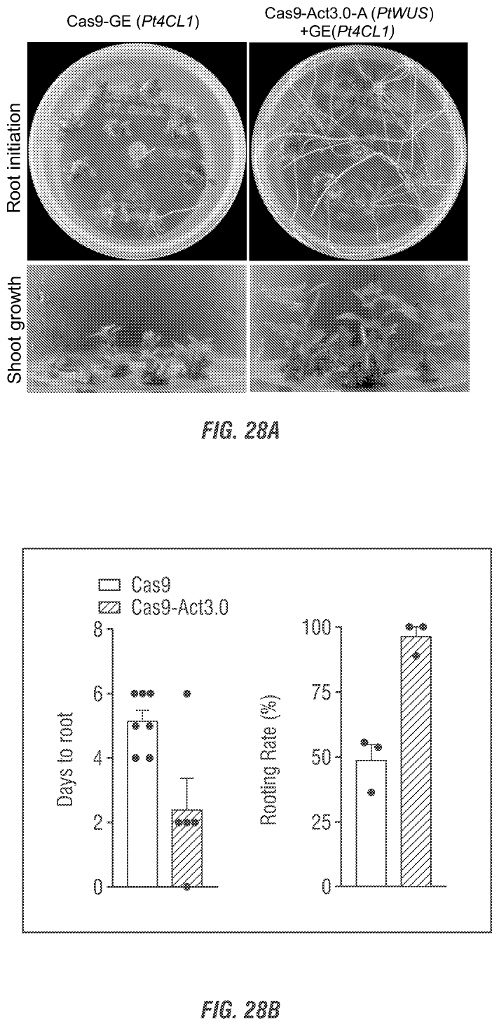

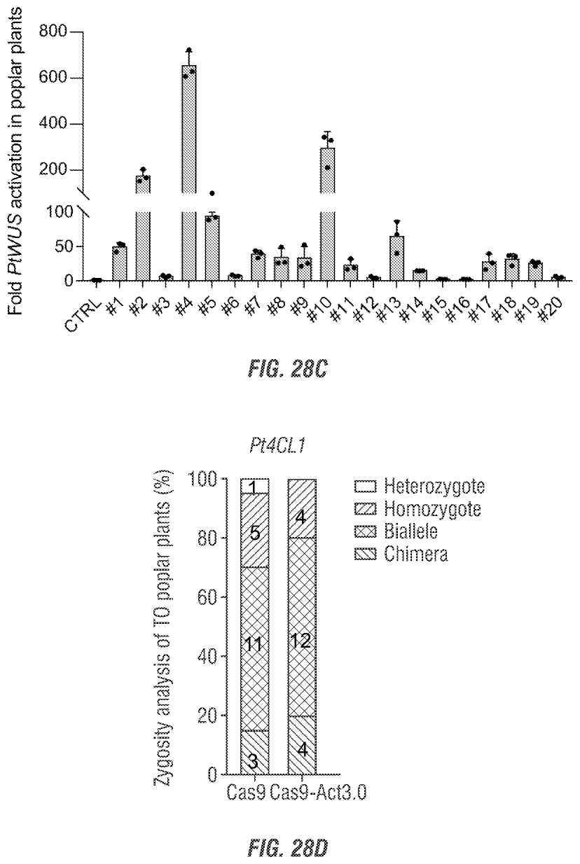

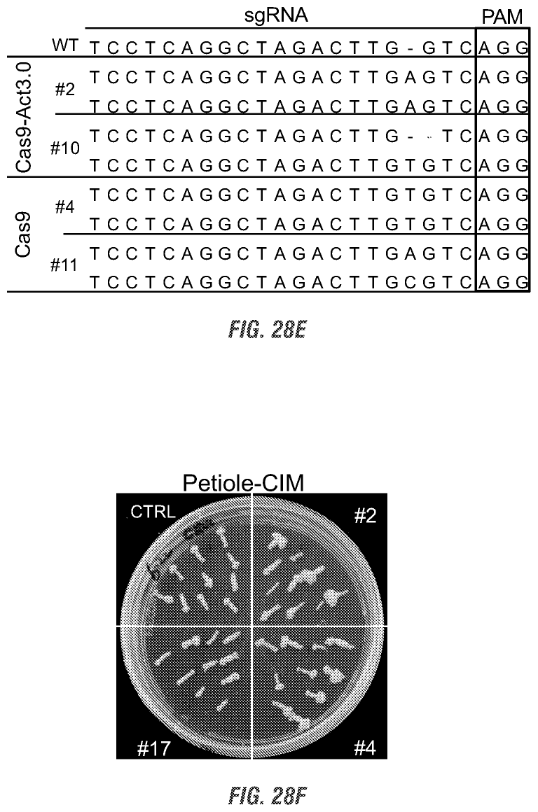

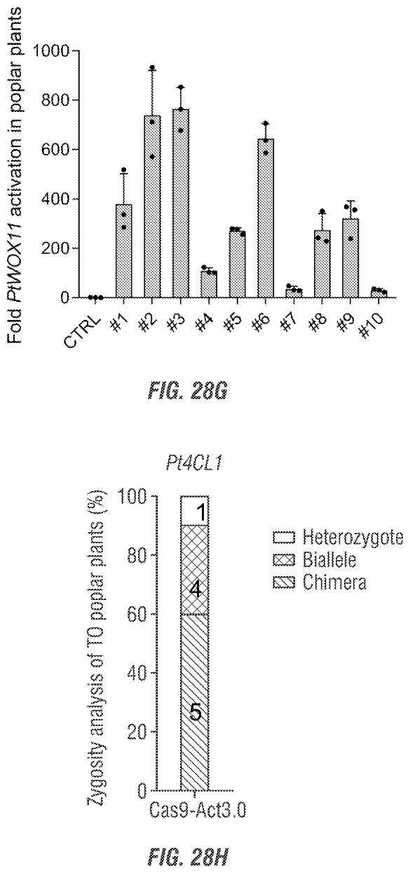

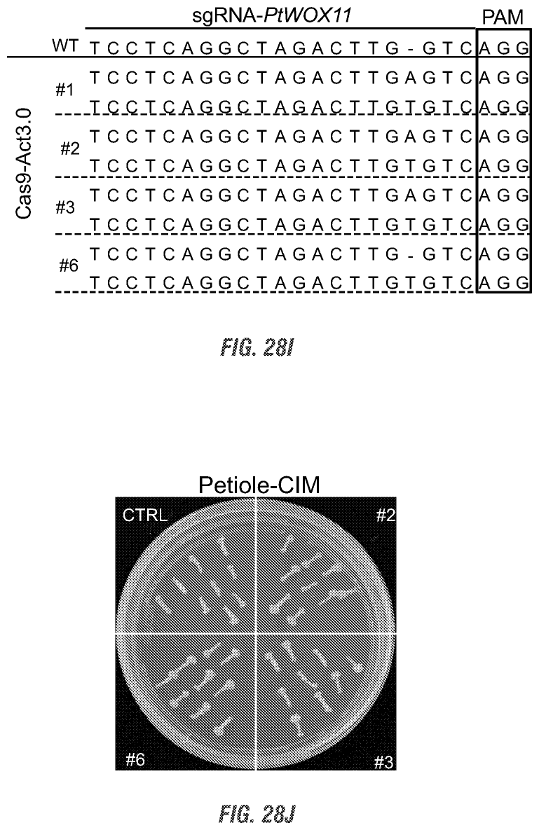

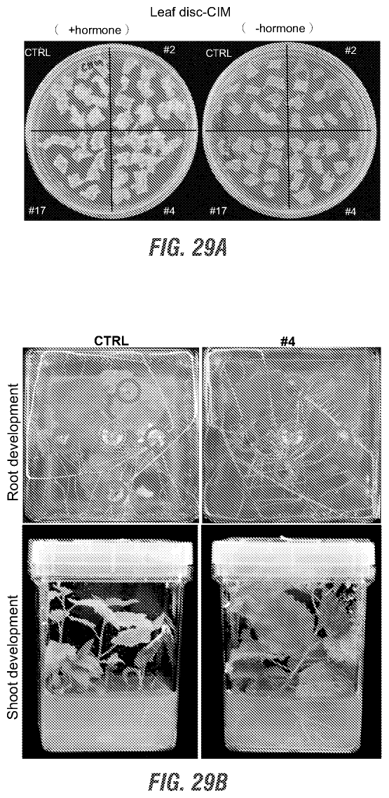

Many plant species are recalcitrant for tissue culture and regeneration. Even a plant species can be regenerated, the process is often lengthy and tedious. These challenges prevent the wide use of genome editing in many plant species. To overcome this challenge, ectopic expression of morphogenic genes was successful applied to boost plant tissue culture and de novo meristem regeneration. The nature of pluripotency of plant cells and the presence of morphogenic genes in every plant genome led us to hypothesize that plant regeneration could be stimulated by activation of endogenous morphogenic genes. Hence, we explored the idea of promoting plant tissue culture in genome editing experiments with CRISPR-Combo. We conducted targeted mutagenesis of the Pt4CL1 gene in poplar, a model of a woody plant. one uses Cas9 for genome editing (Cas9-GE), and the other employed the CRISPR-Combo system to simultaneously edit Pt4CL1 and activate the poplar WUSCHEL gene (PtWUS). Compared to the conventional Cas9-GE construct, the CRISPR-Combo construct (Cas9-Act3.0-A+GE) resulted in rapid tissue culture with accelerated root initiation and shoot growth (FIG. 28A). With CRISPR-Combo, the days to root were reduced in half while the rooting rate was doubled to nearly 100% (FIG. 28B). Expression analysis by qRT-PCR on 20 CRISPR-Combo T0 lines showed PtWUS activation in most lines, with hundreds of fold activation in multiple lines (FIG. 28C). Remarkably, both Cas9 and CRISPR-Combo systems resulted in 100% genome editing efficiency, and over 75% of lines carried homozygous or biallelic mutations for either construct (FIG. 28D-E). We further tested three CRISPR-Combo lines with various levels of PtWUS activation on the callus induction medium (CIM). Interestingly, the lines (#2 and #4) with 200-fold or higher PtWUS activation showed the rapid de-novo callus induction of petiole, when compared to the low activation line (#17) or the control line (FIG. 28F). We also conducted de-novo callus regeneration from leaf disks and more callus was induced from leaf disks of PtWUS high activation lines (#2 and #4) (FIG. 29A). Furthermore, high PtWUS activation line #4 showed more robust de-novo root initiation and shoot growth of stem cuttings and the resulting in vitro cultured plants outgrew the controls in size and biomass (FIG. 29B).

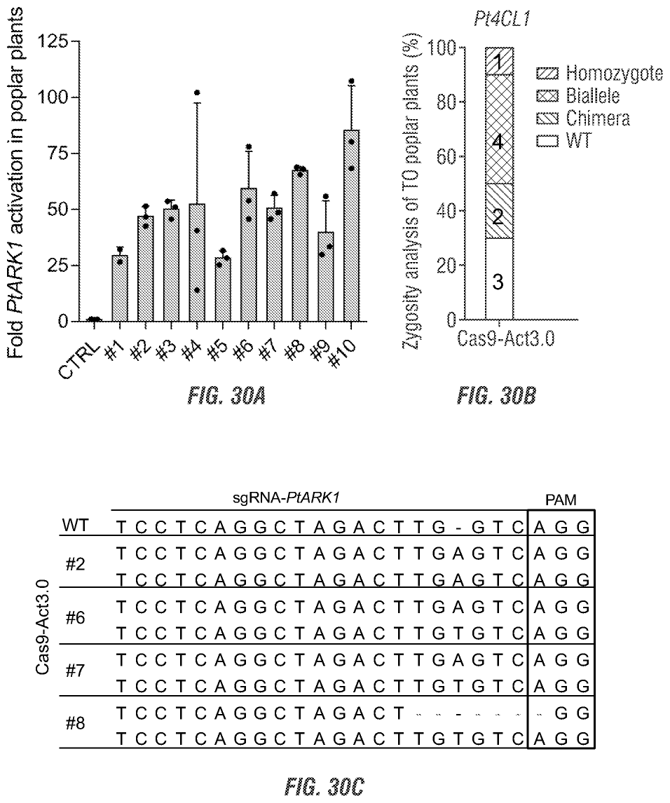

We decided to assess the CRISPR-Combo system for simultaneous editing of Pt4CL1 and activation of other morphogenic genes. In one case, we targeted PtWOX11 for activation. Analysis of 10 randomly chosen CRISPR-Combo T0 lines showed a high level of PtWOX11 activation, up to 800-fold (FIG. 28G). Importantly, Pt4CL1 was edited in all these 10 T0 lines, with one heterozygous mutant, four biallelic mutants, and five mosaic mutants (FIG. 28H). Analysis of the four biallelic mutants showed mutations of lbp deletions and insertion of A or T at the target site (FIG. 281). Interestingly, like the PtWUS high activation lines, the lines (#2, #3 and #6) with 600-fold or higher PtWOX11 activation also showed the rapid de-novo callus regeneration from petioles (FIG. 28J). In another case, we targeted PtARK1 for activation. Analysis of 10 CRISPR-Combo T0 lines showed PtARK1 activation in all lines, ranging from 25-fold to 80-fold (FIG. 30A). Again, all these lines were genome-edited at the Pt4CL1 site (FIG. 30B-C). These results suggest CRISPR-Combo can be robustly used for simultaneous genome editing and morphogenic gene activation in poplar.

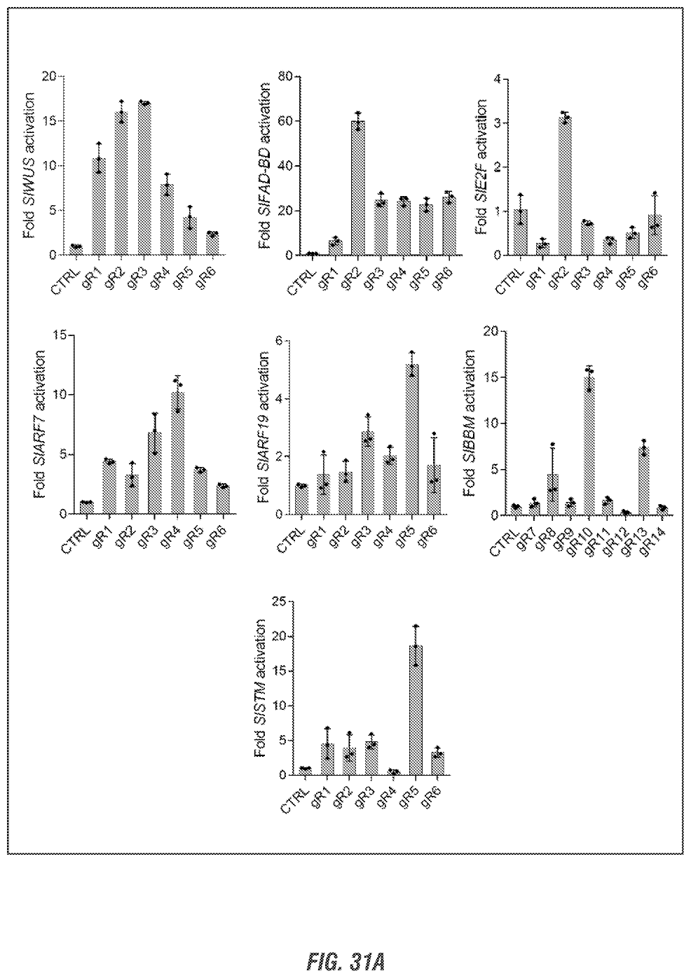

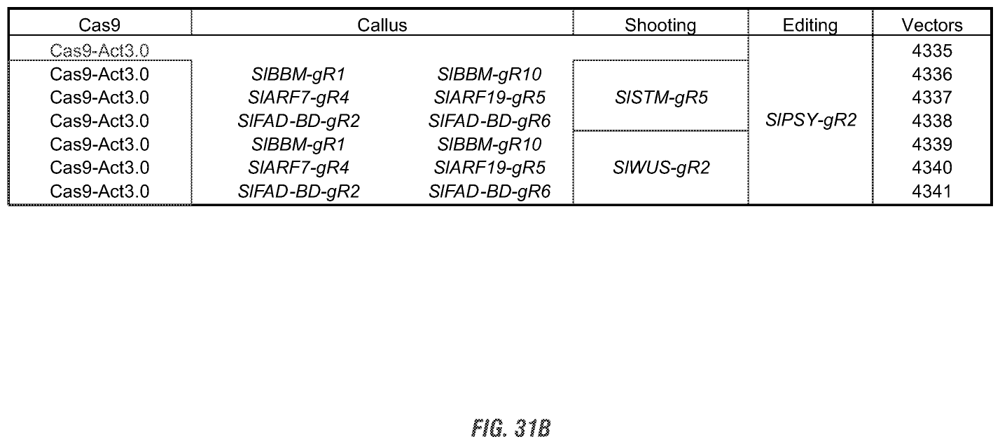

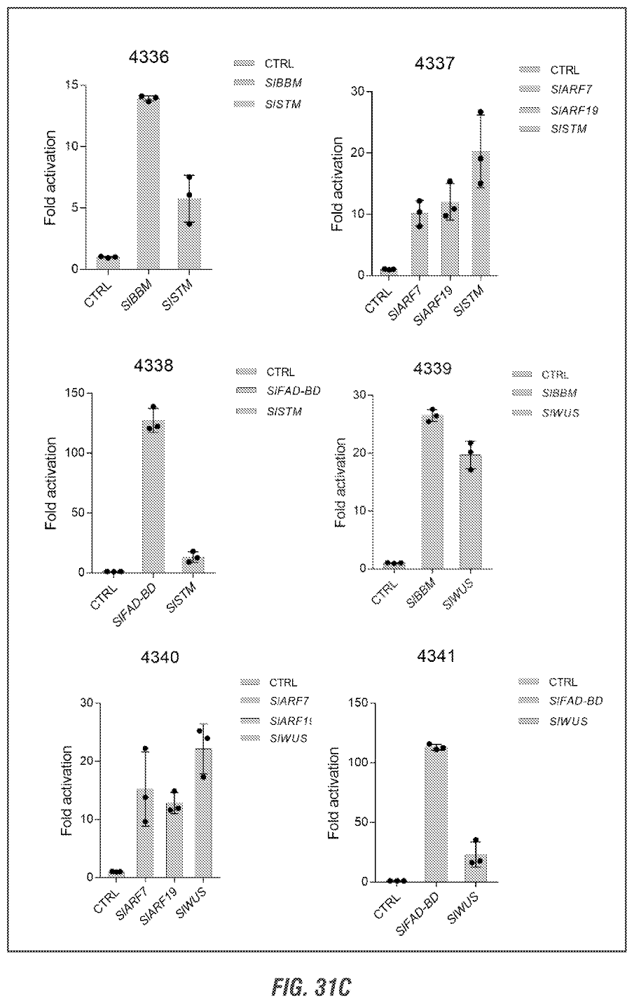

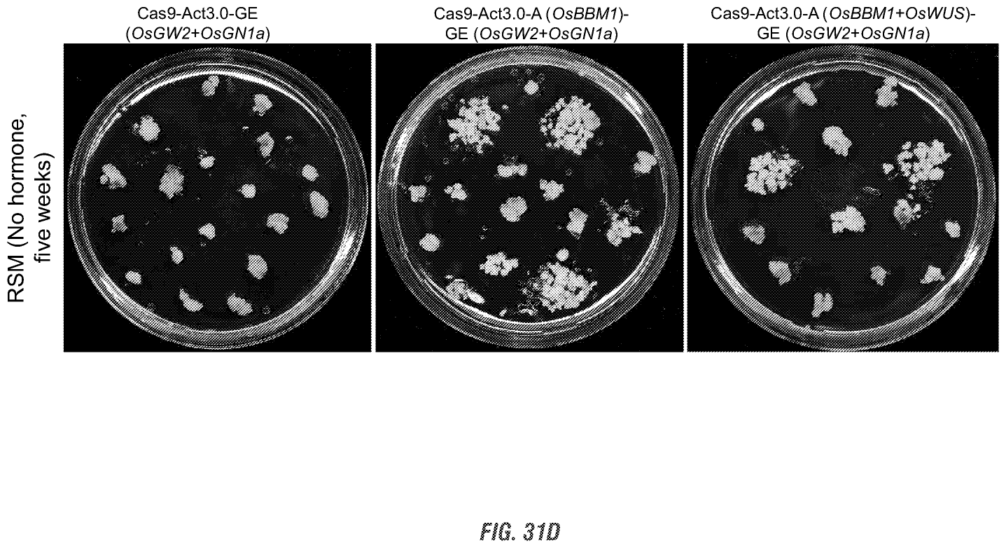

To test CRISPR-Combo for morphogen activation in another plant species, we chose tomato and selected seven morphogenic genes, including SlWUS, SlFAD-BD, SlE2F, SlARF7, SlARF19, SlBBM, and SlSTM, which are involved in callus formation and soot meristem development. For each gene, we screened multiple sgRNAs and identified those that can mount high levels of gene activation for these individual genes (FIG. 31A). To see whether a CRISPR-Combo construct could simultaneously activate more than one morphogenic gene, we designed six multiplexed CRISPR-Combo constructs that can activate two to three morphogenic genes at once while editing the SlPSY gene in tomato (FIG. 31B). Analysis in tomato protoplasts showed that these target morphogenic genes were indeed activated by the corresponding CRISPR-Combo constructs. Hence, the CRISPR-Combo based approach for activation of endogenous morphogenic genes while inducing genome editing can be widely used in plants.

Having demonstrated the use of CRISPR-Combo for boosting tissue culture in dicot plants, we next pursued enhancing tissue culture with CRISPR-Combo in a monocot crop, rice. Previous studies have reported improvements in tissue culture of monocot plants by overexpression of WUSCHEL (WUS) and BABY BOOM (BBM) sourced from maize (Lowe et al., Plant Cell (2016) 28:1998-2015). We hypothesized that activation of OsBBM1 along or together with OsWUS would promote rice tissue culture independent of exogenous plant hormones. The EHA105:Cas9-Act3.0-GE strain-infected control callus explants couldn't produce any hygromycin-resistant calluses on hormone (2, 4-D)-free regeneration and selection medium (RSM). However, about 20% of callus explants infected with EHA105:Cas9-Act3.0-A-GE strains with activation of OsBBM1 or OsBBM1 & OsWUS1 showed hygromycin resistant callus growth (FIG. 31D). These data suggest activation of OsBBM1 alone is sufficient to promote rice transformation. Hence, we demonstrated plant hormone-free plant regeneration in rice with CRISPR-Combo.

### Discussion for Examples 6-9

We built a novel CRISPR-Combo platform to unleash versatile genome engineering in plants. We developed CRISPR-Combo systems for simultaneous targeted mutagenesis by NHEJ and gene activation, as well as simultaneous base editing and gene activation. While there are numerous applications, we focused on the demonstration of CRISPR-Combo for enabling plant genome editing experiments. In one example, we showed that CRISPR-Combo facilitated accelerated breeding and selection of transgene-free genome-edited plants through simultaneous activation of the endogenous florigen, FT. Although the concept was demonstrated in a model plant Arabidopsis, the technology is readily transferable into crop plants. In a second example, we directly worked on an application of CRISPR-Combo in the bioenergy crop poplar. We showed that by simultaneous activation of endogenous morphogenic genes such WUS, regeneration of genome-edited polar plants could be accelerated. Based on our data, it is conceivable that activation of florigen genes and morphogenic genes may be combined to further fast-track the breeding of transgene-free genome-edited crops. Hence, CRISPR-Combo greatly contributes to the improvement and application of genome editing in plants.

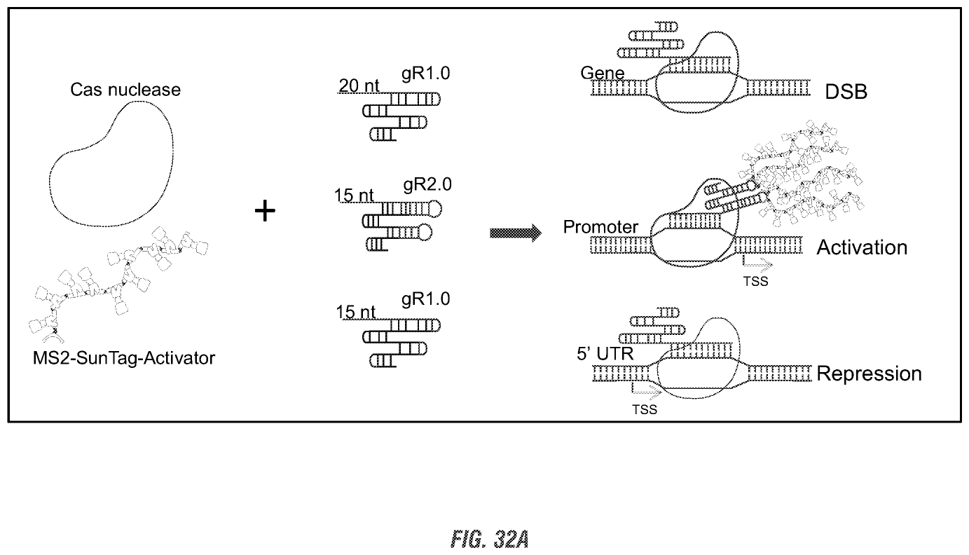

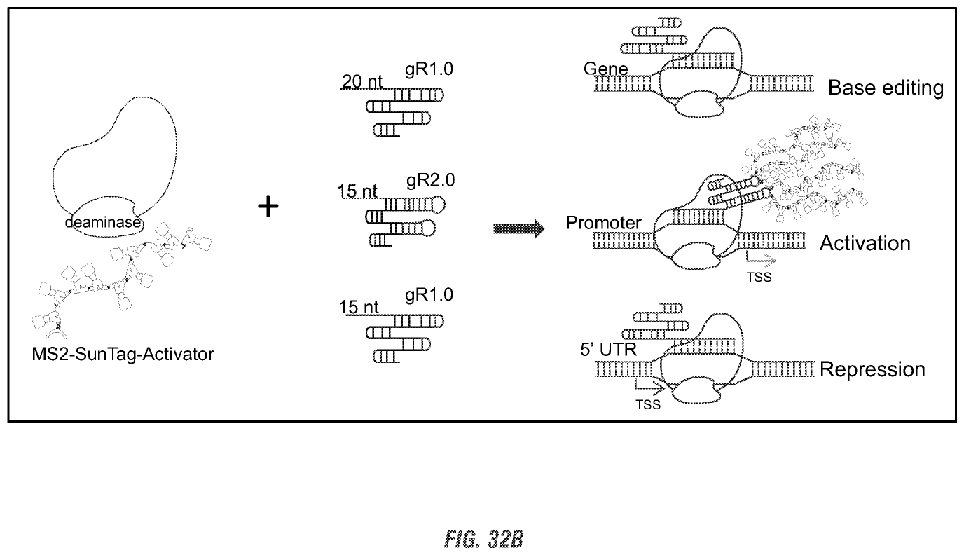

Previously, simultaneous genome editing and gene regulation was demonstrated using orthogonal Cas9 systems that require the expression of multiple Cas9 proteins sourced from different bacteria. By contrast, CRISPR-Combo is based on a single Cas9, which can be used for simultaneous genome editing, gene activation, and gene repression (FIG. 32). Incredibly, programming these three distinct functionalities is simply through picking different sgRNA scaffolds and protospacer lengths (FIG. 32). Hence, practicing CRISPR-Combo is as easy as working with any multiplexed CRISPR system. Because CRISPR-Combo allows for simultaneous modifications of the genome and transcriptome in the organism, it will open new frontiers in plant genome engineering, metabolic engineering, and synthetic biology. The powerful demonstrations shown in this study are only the tip of the iceberg for the full potential of CRISPR-Combo. Finally, the concept and principle of CRISPR-Combo can be broadly applied to other eukaryotic organisms beyond plants to empower simultaneous genome engineering and cell programming.

### Example 10: The CRISPR-Combo System for Simultaneous Gene Repression in Plants

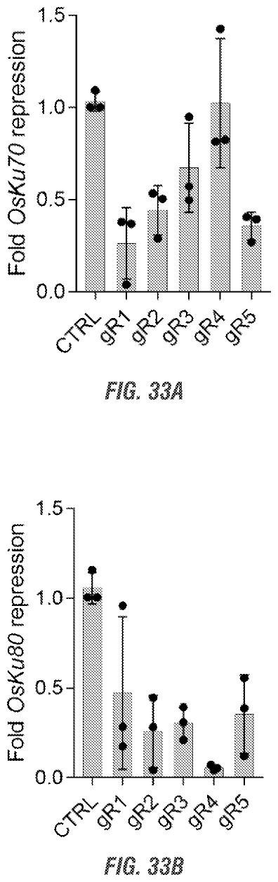

We pursued CRISPR interference (CRISPRi) for OsKU70 and OsKU80, both of which are involved in the canonical non-homologous end joining (NHEJ) DNA repair pathway in rice. For OsKu70, four of five sgRNAs (gR1 to gR5) combined with dCas9 resulted in predominantly gene repression. In particular, the gR1 reduced the OsKu70 expression level by 73.7%. Similarly, all five sgRNAs (gR1 to gR5) resulted in significant gene repression of OsKu80. The gR4 reduced the OsKu80 expression level by 94.5% (FIG. 33). These data suggest that CRISPR-mediated binding near the transcription start site (TSS) can repress gene transcription by blocking RNA polymerase activity. These results further support the feasibility of using CRISPR-Combo systems for simultaneous gene editing, activation, and repression. By targeting the TSS with a gR1.0 with 15 nt sgRNA, the Cas9 derived from the CRISPR-Combo system could repress target gene transcription by blocking RNA polymerase activity. Furthermore, suppression of the nonhomologous end-joining (NHEJ) key molecules KU70 or KU80 expression would allow us to improve the frequencies of homology-directed repair (HDR) and precise gene modifications.

What is claimed is:

1. A system for activating expression of a target nucleic acid, the system comprising:
(i) a Cas polypeptide, or a polynucleotide encoding the Cas polypeptide;
(ii) a guide polynucleotide comprising an aptamer;
(iii) a polypeptide comprising an adapter domain and a multimerized epitope, wherein the adapter domain binds the aptamer, or a polynucleotide encoding the polypeptide; and
(iv) a polypeptide comprising an affinity domain and a transcriptional activation domain, wherein the affinity domain binds the multimerized epitope, or a polynucleotide encoding the polypeptide.

2. The system of claim 1, wherein the Cas polypeptide is a Cas9 or a Cas12b.

3. The system of claim 1, wherein the guide polynucleotide is a dead guide polynucleotide.

4. The system of claim 1, wherein the aptamer is an MS2 aptamer, and wherein the adapter domain comprises an MS2 bacteriophage coat protein (MCP).

5. The system of claim 1, wherein the multimerized epitope comprises a GCN4 epitope.

6. The system of claim 5, wherein the multimerized epitope comprises from about 2 copies of the GCN4 epitope to about 10 copies of the GCN4 epitope.

7. The system of claim 6, wherein the multimerized epitope comprises 10 copies of the GCN4 epitope.

8. The system of claim 1, wherein the affinity domain comprises scFv.

9. The system of claim 1, wherein the transcriptional activation domain comprises a TAL Activation Domain (TAD), two repeats of TAD (2xTAD), 2xTAD-VP64, TV, VPR, or VP64.

10. The system of claim 9, wherein the transcriptional activation domain comprises 2xTAD.

11. The system of claim 1, wherein the Cas polypeptide is a nuclease deficient Cas polypeptide.

12. The system of claim 1, wherein the Cas polypeptide is a nuclease active Cas polypeptide.

13. The system of claim 1, wherein the Cas polypeptide is fused to a transcriptional activation domain.

14. The system of claim 1, wherein the Cas polypeptide is not fused to a transcriptional activation domain.

15. The system of claim 1, wherein components (i)-(iv) are located on one or more vectors.

16. A system for simultaneously activating expression of a target nucleic acid and modifying a nucleotide sequence at a target site in a genome, the system comprising:
(i) a Cas polypeptide, or a polynucleotide encoding the Cas polypeptide;
(ii) a dead guide polynucleotide that mediates increased expression of a target nucleic acid, wherein the dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide; and
(v) a guide polynucleotide that mediates sequence-specific cleavage at a target site in the genome.

17. The system of claim 16, wherein the Cas polypeptide is a Cas9 or a Cas12b.

18. The system of claim 16, wherein the aptamer is an MS2 aptamer, and wherein the adapter domain comprises an MCP.

19. The system of claim 16, wherein the multimerized epitope comprises 10 copies of a GCN4 epitope.

20. The system of claim 16, wherein the affinity domain comprises scFv.

21. The system of claim 16, wherein the transcriptional activation domain comprises 2xTAD, 2xTAD-VP64, TV, VPR, or VP64.

22. The system of claim 16, wherein the Cas polypeptide is fused to a deaminase domain.

23. The system of claim 16, wherein components (i)-(v) are located on one or more vectors.

24. A system for simultaneously activating expression of a first target nucleic acid and repressing expression of a second target nucleic acid, the system comprising:
(i) a Cas polypeptide, or a polynucleotide encoding the Cas polypeptide;
(ii) a first dead guide polynucleotide that mediates increased expression of the first target nucleic acid, wherein the first dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide; and
(v) a second dead guide polynucleotide that mediates reduced expression of the second target nucleic acid.

25. The system of claim 24, wherein the Cas polypeptide is a Cas9 or a Cas12b.

26. The system of claim 24, wherein the aptamer is an MS2 aptamer, and wherein the adapter domain comprises an MCP.

27. The system of claim 24, wherein the multimerized epitope comprises 10 copies of a GCN4 epitope.

28. The system of claim 24, wherein the affinity domain comprises scFv.

29. The system of claim 23, wherein the transcriptional activation domain comprises 2xTAD, 2xTAD-VP64, TV, VPR, or VP64.

30. The system of claim 24, wherein components (i)-(v) are located on one or more vectors.

31. A system for simultaneously activating expression of a first target nucleic acid, repressing expression of a second target nucleic acid, and modifying a nucleotide sequence at a target site in a genome, the system comprising:
(i) a Cas polypeptide, or a polynucleotide encoding the Cas polypeptide;
(ii) a first dead guide polynucleotide that mediates increased expression of the first target nucleic acid, wherein the first dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide;
(v) a second dead guide polynucleotide that mediates reduced expression of the second target nucleic acid; and
(vi) a guide polynucleotide that mediates sequence-specific cleavage at a target site in the genome.

32. The system of claim 31, wherein the Cas polypeptide is a Cas9 or a Cas12b.

33. The system of claim 31, wherein the aptamer is an MS2 aptamer, and wherein the adapter domain comprises an MCP.

34. The system of claim 31, wherein the multimerized epitope comprises 10 copies of a GCN4 epitope.

35. The system of claim 31, wherein the affinity domain comprises scFv.

36. The system of claim 31, wherein the transcriptional activation domain comprises 2xTAD, 2xTAD-VP64, TV, VPR, or VP64.

37. The system of claim 31, wherein the Cas polypeptide is fused to a deaminase domain.

38. The system of claim 31, wherein components (i)-(vi) are located on one or more vectors.

39. A plant or a plant cell comprising the system of any one of claims 1-38.

40. A plant or plant cell comprising:
(i) a Cas polypeptide, or a polynucleotide encoding the Cas polypeptide;
(ii) a first dead guide polynucleotide that mediates increased expression of a first target nucleic acid, wherein the first dead guide polynucleotide comprises an aptamer;
(iii) a polypeptide comprising an adapter domain and a multimerized epitope, wherein the adapter domain binds the aptamer, or a polynucleotide encoding the polypeptide; and
(iv) a polypeptide comprising an affinity domain and a transcriptional activation domain, wherein the affinity domain binds the multimerized epitope, or a polynucleotide encoding the polypeptide.

41. The plant or plant cell of claim 40, further comprising:
a second dead guide polynucleotide that mediates reduced expression of a second target nucleic acid; or
a guide polynucleotide that mediates sequence-specific cleavage at a target site in the genome of the plant cell.

42. The plant or plant cell of claim 40, wherein the Cas polypeptide is a Cas9 or a Cas12b.

43. The plant or plant cell of claim 40, wherein the aptamer is an MS2 aptamer, and wherein the adapter domain comprises an MCP.

44. The plant or plant cell of claim 40, wherein the multimerized epitope comprises 10 copies of a GCN4 epitope.

45. The plant or plant cell of claim 40, wherein the affinity domain comprises scFv.

46. The plant or plant cell of claim 40, wherein the transcriptional activation domain comprises 2xTAD, 2xTAD-VP64, TV, VPR, or VP64.

47. The plant or plant cell of claim 40, wherein the Cas polypeptide is fused to a deaminase domain.

48. A method for activating expression of a target nucleic acid in a plant cell, the method comprising:
introducing in the plant cell:
(i) a Cas polypeptide, or a polynucleotide encoding the Cas polypeptide;
(ii) a guide polynucleotide comprising an aptamer;
(iii) a polypeptide comprising an adapter domain and a multimerized epitope, wherein the adapter domain binds the aptamer, or a polynucleotide encoding the polypeptide; and
(iv) a polypeptide comprising an affinity domain and a transcriptional activation domain, wherein the affinity domain binds the multimerized epitope, or a polynucleotide encoding the polypeptide.

49. The method of claim 48, wherein the Cas polypeptide is a Cas9 or a Cas12b.

50. The method of claim 48, wherein the guide polynucleotide is a dead guide polynucleotide.

51. The method of claim 48, wherein the aptamer is an MS2 aptamer, and wherein the adapter domain comprises an MCP.

52. The method of claim 48, wherein the multimerized epitope comprises a GCN4 epitope.

53. The method of claim 52, wherein the multimerized epitope comprises from about 2 copies of the GCN4 epitope to about 10 copies of the GCN4 epitope.

54. The method of claim 53, wherein the multimerized epitope comprises 10 copies of the GCN4 epitope.

55. The method of claim 48, wherein the affinity domain comprises scFv.

56. The method of claim 48, wherein the transcriptional activation domain comprises TAD, 2xTAD, 2xTAD-VP64, TV, VPR, or VP64.

57. The method of claim 56, wherein the transcriptional activation domain comprises 2xTAD.

58. The method of claim 48, wherein the Cas polypeptide is a nuclease deficient Cas polypeptide.

59. The method of claim 48, wherein the Cas polypeptide is a nuclease active Cas polypeptide.

60. The method of claim 48, wherein the Cas polypeptide is fused to a transcriptional activation domain.

61. The method of claim 48, wherein the Cas polypeptide is not fused to a transcriptional activation domain.

62. The method of claim 48, wherein components (i)-(iv) are located on one or more vectors.

63. A method for simultaneously activating expression of a target nucleic acid and modifying a nucleotide sequence at a target site in a genome of a plant cell, the method comprising:
introducing in the plant cell:
(i) a Cas polypeptide, or a polynucleotide encoding the Cas polypeptide;
(ii) a dead guide polynucleotide that mediates increased expression of a target nucleic acid, wherein the dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide; and
(v) a guide polynucleotide that mediates sequence-specific cleavage at a target site in the genome.

64. The method of claim 63, wherein the Cas polypeptide is a Cas9 or a Cas12b.

65. The method of claim 63, wherein the aptamer is an MS2 aptamer, and wherein the adapter domain comprises an MCP.

66. The method of claim 63, wherein the multimerized epitope comprises 10 copies of the GCN4 epitope.

67. The method of claim 63, wherein the affinity domain comprises scFv.

68. The method of claim 63, wherein the transcriptional activation domain comprises 2xTAD, 2xTAD-VP64, TV, VPR, or VP64.

69. The method of claim 63, wherein the Cas polypeptide is fused to a deaminase domain.

70. The method of claim 63, wherein components (i)-(v) are located on one or more vectors.

71. A method for simultaneously activating expression of a first target nucleic acid and repressing expression of a second target nucleic acid in a plant cell, the method comprising:
introducing in the plant cell:
(i) a Cas polypeptide, or a polynucleotide encoding the Cas polypeptide;
(ii) a first dead guide polynucleotide that mediates increased expression of the first target nucleic acid, wherein the first dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide; and
(v) a second dead guide polynucleotide that mediates reduced expression of the second target nucleic acid.

72. The method of claim 71, wherein the Cas polypeptide is a Cas9 or a Cas12b.

73. The method of claim 71, wherein the aptamer is an MS2 aptamer, and wherein the adapter domain comprises an MCP.

74. The method of claim 71, wherein the multimerized epitope comprises 10 copies of a GCN4 epitope.

75. The method of claim 71, wherein the affinity domain comprises scFv.

76. The method of claim 71, wherein the transcriptional activation domain comprises 2xTAD, 2xTAD-VP64, TV, VPR, or VP64.

77. The method of claim 71, wherein components (i)-(v) are located on one or more vectors.

78. A method for simultaneously activating expression of a first target nucleic acid, repressing expression of a second target nucleic acid, and modifying a nucleotide sequence at a target site in a genome of a plant cell, the method comprising:
introducing in the plant cell:
(i) a Cas polypeptide, or a polynucleotide encoding the Cas polypeptide;
(ii) a first dead guide polynucleotide that mediates increased expression of the first target nucleic acid, wherein the first dead guide polynucleotide comprises an aptamer;
(iii) a polypeptide comprising an adapter domain and a multimerized epitope, wherein the adapter domain binds the aptamer, or a polynucleotide encoding the polypeptide;
(iv) a polypeptide comprising an affinity domain and a transcriptional activation domain, wherein the affinity domain binds the multimerized epitope, or a polynucleotide encoding the polypeptide;
(v) a second dead guide polynucleotide that mediates reduced expression of the second target nucleic acid; and
(vi) a guide polynucleotide that mediates sequence-specific cleavage at a target site in the genome.

79. The method of claim 78, wherein the Cas polypeptide is a Cas9 or a Cas12b.

80. The method of claim 78, wherein the aptamer is an MS2 aptamer, and wherein the adapter domain comprises an MCP.

81. The method of claim 78, wherein the multimerized epitope comprises 10 copies of a GCN4 epitope.

82. The method of claim 78, wherein the affinity domain comprises scFv.

83. The method of claim 78, wherein the transcriptional activation domain comprises 2xTAD, 2xTAD-VP64, TV, VPR, or VP64.

84. The method of claim 78, wherein the Cas polypeptide is fused to a deaminase domain.

85. The method of claim 78, wherein components (i)-(vi) are located on one or more vectors.

86. A vector comprising:
(i) a polynucleotide encoding a Cas polypeptide;
(ii) a polynucleotide encoding a polypeptide comprising an adapter domain and a multimerized epitope, wherein the adapter domain is capable of binding an aptamer; and
(iii) a polynucleotide encoding a polypeptide comprising an affinity domain and a transcriptional activation domain, wherein the affinity domain binds the epitope.

87. The vector of claim 86, wherein the Cas polypeptide is a Cas9 or a Cas12b.

88. The vector of claim 86, wherein the adapter domain comprises an MCP capable of binding an MS2 aptamer.

89. The vector of claim 86, wherein the multimerized epitope comprises 10 copies of a GCN4 epitope.

90. The vector of claim 86, wherein the affinity domain comprises scFv.

91. The vector of claim 86, wherein the transcriptional activation domain comprises 2xTAD.

92. The vector of claim 86, wherein the Cas polypeptide is fused to a deaminase domain.

93. The vector of claim 86, wherein the vector comprises the nucleic acid sequence of any of SEQ ID NOs: 37-47.
